SEQUENCE LISTING (44 sequences)

SEQ ID NO: 1 - DNA, 21 bp, Synthetic construct
  source 1..21: mol_type = other DNA
gcggtcggcggggctcttgcg

SEQ ID NO: 2 - DNA, 21 bp, Synthetic construct
  source 1..21: mol_type = other DNA
ggagggggccgcgccggaggc

SEQ ID NO: 3 - DNA, 21 bp, Synthetic construct
  source 1..21: mol_type = other DNA
ccgggaaactggggtcccccg

SEQ ID NO: 4 - DNA, 21 bp, Synthetic construct
  source 1..21: mol_type = other DNA
ggggcccgcgccgaagagggg

SEQ ID NO: 5 - DNA, 21 bp, Synthetic construct
  source 1..21: mol_type = other DNA
ggggagtggctgacacagcgg

SEQ ID NO: 6 - DNA, 21 bp, Synthetic construct
  source 1..21: mol_type = other DNA
gggtgacggagggttctagag

SEQ ID NO: 7 - DNA, 21 bp, Synthetic construct
  source 1..21: mol_type = other DNA
tctctccctctcagtcggcct

SEQ ID NO: 8 - DNA, 7541 bp, Synthetic construct
  source 1..7541: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttatagtactctggaaacagaatctactataacaaggcaaaatgccgtgtttatctcgtcaacttgttggcgagatttttttgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 9 - DNA, 7543 bp, Synthetic construct
  source 1..7543: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcggtcggcggggctcttgcggttatagtactctggaaacagaatctactataacaaggcaaaatgccgtgtttatctcgtcaacttgttggcgagatttttttgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 10 - DNA, 7543 bp, Synthetic construct
  source 1..7543: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggagggggccgcgccggaggcgttatagtactctggaaacagaatctactataacaaggcaaaatgccgtgtttatctcgtcaacttgttggcgagatttttttgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 11 - DNA, 7543 bp, Synthetic construct
  source 1..7543: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccgggaaactggggtcccccggttatagtactctggaaacagaatctactataacaaggcaaaatgccgtgtttatctcgtcaacttgttggcgagatttttttgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 12 - DNA, 7543 bp, Synthetic construct
  source 1..7543: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggggcccgcgccgaagagggggttatagtactctggaaacagaatctactataacaaggcaaaatgccgtgtttatctcgtcaacttgttggcgagatttttttgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 13 - DNA, 7543 bp, Synthetic construct
  source 1..7543: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggggagtggctgacacagcgggttatagtactctggaaacagaatctactataacaaggcaaaatgccgtgtttatctcgtcaacttgttggcgagatttttttgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 14 - DNA, 7543 bp, Synthetic construct
  source 1..7543: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggtgacggagggttctagaggttatagtactctggaaacagaatctactataacaaggcaaaatgccgtgtttatctcgtcaacttgttggcgagatttttttgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 15 - DNA, 7543 bp, Synthetic construct
  source 1..7543: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctctccctctcagtcggcctgttatagtactctggaaacagaatctactataacaaggcaaaatgccgtgtttatctcgtcaacttgttggcgagatttttttgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 16 - DNA, 7460 bp, Synthetic construct
  source 1..7460: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttatagtactctggaaacagaatctactataacaaggcaaaatgccgtgtttatctcgtcaacttgttggcgagatttttttgcgttgacattgattattgactagtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 17 - DNA, 7462 bp, Synthetic construct
  source 1..7462: mol_type = other DNA
cagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccgggaaactggggtcccccggttatagtactctggaaacagaatctactataacaaggcaaaatgccgtgtttatctcgtcaacttgttggcgagatttttttgcgttgacattgattattgactagtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgatctagagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagctagaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggcttccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatgg

SEQ ID NO: 18 - DNA, 241 bp, Homo sapiens
  source 1..241: mol_type = genomic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 19 - DNA, 584 bp, Homo sapiens
  source 1..584: mol_type = genomic DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 20 - DNA, 495 bp, Synthetic construct
  source 1..495: mol_type = other DNA
gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctaga

SEQ ID NO: 21 - DNA, 76 bp, Synthetic construct
  source 1..76: mol_type = other DNA
gttatagtactctggaaacagaatctactataacaaggcaaaatgccgtgtttatctcgtcaacttgttggcgaga

SEQ ID NO: 22 - DNA, 21 bp, Synthetic construct
  source 1..21: mol_type = other DNA
ccaaagaagaagcggaaggtc

SEQ ID NO: 23 - DNA, 3156 bp, Synthetic construct
  source 1..3156: mol_type = other DNA
aagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 24 - protein, 1052 aa, Synthetic construct
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 25 - DNA, 48 bp, Synthetic construct
  source 1..48: mol_type = other DNA
aaaaggccggcggccacgaaaaaggccggccaggcaaaaaagaaaaag

SEQ ID NO: 26 - DNA, 150 bp, Synthetic construct
  source 1..150: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctg

SEQ ID NO: 27 - protein, 50 aa, Synthetic construct
DALDDFDLDMLGSDALDDFDLDMLGSDALDDFDLDMLGSDALDDFDLDML

SEQ ID NO: 28 - DNA, 208 bp, Synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 29 - DNA, 141 bp, Synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 30 - DNA, 130 bp, Synthetic construct
  source 1..130: mol_type = other DNA
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 31 - DNA, 7380 bp, Mus musculus
  source 1..7380: mol_type = genomic DNA
aggcgccgctcgaagccggggccgatgcagctaaaccgtgccgccgtcgccgccgccgctgcgcctgcggagcccccggagccgctgtcccccgcgctggccccggccccggccccccccggccccctcccgcgtagcgcggtcggcggggctcttgcggggggtcagggggggccagggcgccgcgcggagtccccgtgcgctcctctctccgccgggaacagtccgggcccgggcgctagcaccgggatggacggccccggggccagcgccgtggtcgtgcgcgtcggcatcccggacctgcaacaaacgaagtgcctgcgtctggacccaaccgcgcccgtgtgggccgccaagcagcgtgtgctctgcgccctcaatcatagccttcaagacgcgctcaactacgggctattccagcctccctcccggggtcgcgccggcaagttcctggatgaagagcggctcttacaggactacccgcctaacctggacacgcccctgccctatctggagttccgatacaagcggagagtttatgcccagaacctcatagatgacaagcagtttgcaaagctacacacaaaggcaaacctgaagaagttcatggactatgtccagctacacagcacagataaggtggcccgcctgctggacaaggggctggaccccaatttccatgaccctgactcaggagagtgccctctgagccttgcggcacagttggacaacgccactgacctcctgaaggttctccgcaacggcggtgctcatctggacttccggacccgagatgggctgacagccgtccactgtgctacccgccagcggaacgcaggggcattgacgaccctgctggacctgggggcttcgcctgactacaaggacagccgcggcctgacgcccctgtaccatagtgccctagggggcggggatgccctctgttgcgagctgcttctccatgatcatgcacagctggggaccactgatgagaatggttggcaagagatccatcaggcctgtcgctttggacacgtgcagcacctggagcaccttttgttctatggggccaacatgggtgctcagaatgcctcgggaaacacagccctgcacatctgtgccctctacaaccaggagagttgcgcgcgcgtcctgcttttccgtggtgccaacaaggacgtccgcaattacaacagccagacagccttccaggtggccattattgcagggaactttgagcttgccgaggtaatcaagacccacaaagactccgatgtcgtaccattcagggaaacccccagctatgcaaagcgacggcgtctggctggcccgagtggcctggcatccccacggcccttacagcgctcagccagtgatatcaacctgaaaggtgatcagcccgcagcttctccagggcccactctccgaagcctccctcatcaactcttgctccagaggcttcaggaggagaaagaccgtgacagggatggtgaactggagaatgacatcagcggcccctcagcaggcaggggtggccacaacaagatcagccccagtgggcccggcggatccggccccgcgcccggccccggcccggcgtctcccgcgccccccgcgccgccgccccggggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagaagaaaaggggagaagttggatgagatcctggcagttgccgcggagccgacactgaggccggacattgcagatgctgactcgagggcggccactgtcaagcagcggcccaccagccggaggatcacccctgctgagatcagctcattgtttgagcgccagggcctcccaggcccagagaagctgccgggctctctgcggaaggggattccacggaccaaatctgtaggggaggatgagaagctggcatccctactggaagggcgcttcccacgcagcacgtcaatgcaggacacagtgcgtgaaggtcgaggcattccacccccgccgcagaccgccccgccacccccacccgcgccctactacttcgactccgggccaccccccaccttctcaccgccgccaccaccgggccgggcctatgacactgtgcgctccagcttcaaaccaggcctggaggctcgtctgggtgcgggggccgccggcctgtatgatccgagcacgcctctgggcccgctgccctaccctgagcgtcagaagcgtgcgcgctccatgatcatactgcaggactctgcgccagaagtgggtgatgtcccccggcctgcgcctgccgccacaccgcctgagcgccccaagcgccggcctcggccgtcaggccctgatagtccctatgccaacctgggcgccttcagtgccagcctcttcgctccgtcgaaaccacagcgccgcaagagcccgctggtgaagcagcttcaggtggaggacgctcaggagcgcgcggccctggccgtgggtagcccgggaccagtgggcggaagctttgcacgagaaccctctccgacgcaccgcgggccccggccaggcagccttgactacagctctggagaaggcctaggccttaccttcggcggccctagccctggcccagtcaaggagcggcgcctggaggagcgacgccgttccactgtgttcctctctgtgggtgccatcgagggcagccctcccagcgcggatctgccatccctacaaccctcccgctccattgatgagcgcctcctggggacaggcgccaccactggccgcgatttgctactcccctcccctgtctctgctctgaagccattggtcggtggtcccagccttgggccctcaggctccaccttcatccaccctctcactggcaaacccttggatcctagctcacccttagctcttgctctggctgcccgggagcgggctctggcctcgcaaacaccttcccggtcccccacacctgtgcacagccccgatgctgaccgccctggacccctctttgtggatgtgcaaacccgagactctgagagaggaccgttggcttccccagccttctcccctcggagtccagcgtggattccagtgcctgctcggagagaggcagagaagccccctcgggaagagcggaagtcaccagaggacaagaagtccatgatcctcagcgtcttggacacgtccttgcaacggccagctggcctcattgttgtgcatgccaccagcaatgggcaggagcccagcaggctgggggctgaagaggagcgccccggtactccggagctggccccagcccccatgcaggcagcagctgtggcagagcccatgccaagcccccgggcccagccccctggcagcatcccagcagatcccgggccaggtcaaggcagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctgacgccccactccctctcctgttcctgctgcgccctgccggcagggcccccacccctactccaggccgcaggctcggctcgccccctaccacggcgcccgggccaggaatgttgcatgaatcgtcctgtttgctgttgcttggagacttgccctgtacattgcttagtgccctcccctgccgctgaaccccacccagcacacagtaagggcgcggaccaggggggctgggtggaagggggtgggggcagggtgctctggcctgaccacttcctccatagctcctggtggccattcttccagagggggaacctagtccagcatgcgaggtcaggacacgccttggtgactcggggggaggggggagacattggggttctcgataggggccaaggagccccctgttttacatattttaatccactctatatttggaaagagaaaaggaacaaatatctctgtccgtaatagttcccgccctcttcccctctagtcctctcgctggtcccgccacagctgcccagtcttccatctctggcccctcactgccaccccatacagggcaggggacactccagctggcctggggttagccagggtcctggcagcccaccctggggaccccggctcagccccccttccctcgctgagctatagtatgccccacccaccctttaggtgctgctcagggggacgggtggcaggcattgcctgctgggcactagcagggccaggtggcctgggaaattattgccctggggctgggccccggtaacccaaaccccagccatcatcttcacagggtctctcccacaggaggggtctaacctttcccacttcttgggcaactacagcagagaagcctccctgcctcgcaccccaaagactccccaattcctgccctgtgtgtgtgtgtgcgccacataatgtgtgtgcacgcctgtgtgcttgtgaaaactgggtgtggctgagcgcatgggtgccctgtgtgtgcttgactgtgcagtggtccccaggggctgttctggatgggtgggaggtggcggcagaagcttgcacagggtgcatgcatgggtgtgtgcccgggaaggggccctgtcctgctggtgggtcctgctgttctctcagcctgtcccctcctccccaaacctcacccagcttaagcagggattcttcgtgaatcctttcagcttcgggaggcctcaagggctccccccgcaggcagtaccccttcgggcttctaagggaattgtggggaccactaaaatcaggccacaacagcccttggagagaggcaaagactcctgagggtcccctggcccccttactgtgactcctcacactcagcaatgacctgtggggtggggggccctggggcatttttaaacatcgggtttggagtctggactaagctccatccacgtcactcacaagtttctgtttctatttctagctttttttaataaaaaataaataaataaataaatatataaataaatatatatatatataaaagacagaaaacaggtgttttcatggcccaggggcttggcacgccggtctgtgcccacctgccccaccctggcccatcggccccattccttagacacagagtcacacccactaaccctctcaccaacagagcaggtcacacacacagcagcagtcactgtaacagactgccacatacacagtctcacatttacctgtgggtttttggttctgttcaatttgggtttttaactttacagggtcagttccgcttctcccccccccccttttgtatggagttccatctggggggctttcaaccccctgctccagtcctgaggcctcctgaccctgacgttgtgatacaccccacagagatctatgtttcttatattattattattaataataattattataatattatgtaataaatttataagaaatgaaa

SEQ ID NO: 32 - protein, 1804 aa, Mus musculus
MQLNRAAVAAAAAPAEPPEPLSPALAPAPAPPGPLPRSAVGGALAGGQGGPGRRAESPCAPLSAGNSPGPGASTGMDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGDQPAASPGPTLRSLPHQLLLQRLQEEKDRDRDGELENDISGPSAGRGGHNKISPSGPGGSGPAPGPGPASPAPPAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRRRKGEKLDEILAVAAEPTLRPDIADADSRAATVKQRPTSRRITPAEISSLFERQGLPGPEKLPGSLRKGIPRTKSVGEDEKLASLLEGRFPRSTSMQDTVREGRGIPPPPQTAPPPPPAPYYFDSGPPPTFSPPPPPGRAYDTVRSSFKPGLEARLGAGAAGLYDPSTPLGPLPYPERQKRARSMIILQDSAPEVGDVPRPAPAATPPERPKRRPRPSGPDSPYANLGAFSASLFAPSKPQRRKSPLVKQLQVEDAQERAALAVGSPGPVGGSFAREPSPTHRGPRPGSLDYSSGEGLGLTFGGPSPGPVKERRLEERRRSTVFLSVGAIEGSPPSADLPSLQPSRSIDERLLGTGATTGRDLLLPSPVSALKPLVGGPSLGPSGSTFIHPLTGKPLDPSSPLALALAARERALASQTPSRSPTPVHSPDADRPGPLFVDVQTRDSERGPLASPAFSPRSPAWIPVPARREAEKPPREERKSPEDKKSMILSVLDTSLQRPAGLIVVHATSNGQEPSRLGAEEERPGTPEAPAPMQAAAVAEPMPSPRAQPPGSIPADPGPGQGSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 33 - DNA, 7691 bp, Homo sapiens
  source 1..7691: mol_type = genomic DNA
acacaccgcactggaggctccgctctgcccgctccgggaccccttcctccgccgcacccgccccggtggtccgcggatgcccgcccttgccgctcagccactccccccgcaccgaggcctaggactcccccccccaaccccgtcacagccccccagacccccgccccgtggctcggcccccgccctccgcacacacctcccgcccccacccgggaccccgcaagtaaccccccagcactggccctgagccctcccggcccccgcctccggcgcagccccctcgccacccccgcttccctcccgtctcaggccccctccccccgccgcccccgcccccggggaaggcaggcgccgagctgagccggggccgatgcagctgagccgcgccgccgccgccgccgccgccgcccctgcggagcccccggagccgctgtcccccgcgccggccccggccccggccccccccggccccctcccgcgcagcgcggccgacggggctccggcgggggggaagggggggccggggcgccgcgcggagtccccgggcgctccgttccccggcgcgagcggccccggcccgggccccggcgcggggatggacggccccggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggatcaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttgtaccattcagggaaacccccagctatgcgaagcggcggcgactggctggccccagtggcttggcatcccctcggcctctgcagcgctcagccagcgatatcaacctgaagggggaggcacagccagcagcttctcctggaccctcgctgagaagcctcccccaccagctgctgctccagcggctgcaagaggagaaagatcgtgaccgggatgccgaccaggagagcaacatcagtggccctttagcaggcagggccggccaaagcaagatcagcccgagcgggcccggcggccccggccccgcgcccggccccggccccgcgccccctgcgccccccgcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagaagaaaaggggagaagctggacgagatgctggcagccgccgcagagccaacgctgcggccagacatcgcagacgcagactccagagccgccaccgtcaaacagaggcccaccagtcggaggatcacacccgccgagattagctcattgtttgaacgccagggcctcccaggcccagagaagctgccgggctccttgcggaaggggattccacggaccaagtctgtaggggaggacgagaagctggcgtccctgctggaagggcgcttcccgcggagcacctcgatgcaagacccggtgcgcgagggtcgcggcatcccgcccccgccgcagaccgcgccgcctcccccgcccgcgccctactacttcgactcggggccgcccccggccttctcgccgccgcccccgccgggccgcgcctacgacacggtgcgctccagcttcaagcccggcctggaggcgcgcctgggcgcgggcgctgccggcctgtacgagccgggcgcggccctcggcccgctgccgtatcccgagcggcagaagcgcgcgcgctccatgatcatcctgcaggactcggcgcccgagtcgggcgacgcccctcgacccccgcccgcggccaccccgcccgagcgacccaagcgccggccgcggccgcccggccccgacagcccctacgccaacctgggcgccttcagcgccagcctcttcgctccgtccaagccgcagcgccgcaagagccccctggtgaagcagctgcaggtggaggacgcgcaggagcgcgcggccctggccgtgggcagccccggtcccggcggcggcagcttcgcccgcgagccctccccgacccaccgcggtccgcgcccgggtggcctcgactacggcgcgggcgatggcccggggctcgcgttcggcggcccgggcccggccaaggaccggcggctggaggagcggcgccgctccactgtgttcctgtccgtgggggccatcgagggcagcgcccccggcgcggatctgccatccctacagccctcccgctccatcgacgagcgcctcctggggaccggccccaccgccggccgcgacctgctgctgccctccccggtgtctgccctgaagccgttggtcagcggcccgagcctggggccctcgggttccaccttcatccacccactcaccggcaaacccctggaccccagctcacccctggcccttgccctggctgcccgagagcgagctctggcctcccaggcgccctcccggtcccccacacccgtgcacagtcccgacgccgaccgccccggacccctgtttgtggatgtacaggcccgggacccagagcgagggtccctggcttccccggctttctccccacggagcccagcctggattcctgtgcctgctcgcagggaggcagagaaggtcccccgggaggagcggaagtcacccgaggacaagaagtccatgatcctcagcgtcctggacacatccctgcagcggccagctggcctcatcgttgtgcacgccaccagcaacgggcaggagcccagcaggctggggggggccgaagaggagcgcccgggcaccccggagttggccccggcccccatgcagtcagcggctgtggcagagcccctgcccagcccccgggcccagccccctggtggcaccccggcagacgccgggccaggccagggcagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctgacgccccacccccactcccgccccggccgtgccctgccggcagggccccccacccccaccccgggccgcgggctcggcctgccccttacgacggcgcccgggccaggaatgttgcatgaatcgtcctgtttgctgttgctcggagactcgccctgtacattgcttagtgccctcaccggccgcccagcccacccagcgcacagtcaggaagggcgtggaccagggaggctggggcgggaggtgccgggggtggggtgccctagcgtgaccacctccttcgcagctcctggtggccattctcccagagggggaacctagtccagcatgcgaggtcaggacccgccttggtgactcggggggaggggggagacattgggattctcgatgggggccaaggagcccccctgttttgcatattttaatccactctatatttggaacgagaaaaggaacaaatatctctgtccgtaatagtttcctctcccctcccttctacttccactggtcccactgcagctgcccagtcttccatctccggcccctcactgccactgccaccccacaacggggcaggggacgctccagctggtctggggttggccagggccctagtggcccgccctggggccccagctcggcccctcgcctcgctgagctctagtgtgccccaccgacccttcaggtgctgctcgtggtgggaggggcggcaggccgcgggtcctgctgtgcacccgcgggaccagccggcctgggagaccatcggccgggggggatgagggcagggccctgccgctccaccgcagccatcttcctcacagggtctctccccaaggagggggctagcttggtccccatgctcttgggcaactacagcagagaagcctccctgccttggaccccaaagtctcctgtcctgccctttatgtgtgtgggtgaaactgggtgcgtctgagcacgtgggagccgtgtgtgtgcctgattactgagtggccaccaggggccgctctggactagcgcggggccgtggaggcgtgcaccgtgtgcatgcgtggggtgtacctgtgagagcaccctgtctcctcttccaaagaaagtcagaggccatcctgcaccctgggtccagctgtttgcccagcctgtccttccagagcctcacccagcctgagcggggttccctggtgaatccctgctgcttggggaggccccaagggccccttggaggcagcgcccccaccttgggcttctgagggcatcatagggggacccctagagtcagttcaccacaggccctggggagagtcaaagacccccgagggtgcccagccccccacactgtgactcctcacactcagcgatgacctgtggggtggggggccctgggacgtttttaaacctagggtttggagtctggactaagctccatccacgtcactcacaagtttctgtttatatttctagctttttttaataaaataaaaaaaaaaagaaaacagaagttttcacaacccaggggcctggcacgccggtctgtgcctgcccgccccgccctggcccaccggccccactccctgggcacagagtcacacccactcatccttccgccaacagtccaggtcacacagcagcagtcactgtaacagactgccacatacacactcggtctcacactcacctgtgggttttggttccgttcaatttgggtttttaactttacagggtcagttccgcttcacctccttttgtatggagttccatccggggggtttcaccccctgctccagtcctgaggcctcctgaccctgacgttgtgatacgccccacagagatctatgtttcttatattattattattgataataattattataatattattatgtaataaatttataagaaatgaa

SEQ ID NO: 34 - protein, 1806 aa, Homo sapiens [sequence omitted]

SEQ ID NO: 35 - DNA, 19 bp, Synthetic construct
  source 1..19: mol_type = other DNA
ccggacctgcaacaaacga

SEQ ID NO: 36 - DNA, 21 bp, Synthetic construct
  source 1..21: mol_type = other DNA
gcgcgtcttgaaggctatgat

SEQ ID NO: 37 - DNA, 21 bp, Synthetic construct
  source 1..21: mol_type = other DNA
ccgctacaagacccgagtcta

SEQ ID NO: 38 - DNA, 23 bp, Synthetic construct
  source 1..23: mol_type = other DNA
cctgaatctgagtcgtggtagtt

SEQ ID NO: 39 - DNA, 21 bp, Synthetic construct
  source 1..21: mol_type = other DNA
agaggccccagcttattccaa

SEQ ID NO: 40 - DNA, 21 bp, Synthetic construct
  source 1..21: mol_type = other DNA
caggggtatagcttccaaggc

SEQ ID NO: 41 - DNA, 19 bp, Synthetic construct
  source 1..19: mol_type = other DNA
catggccttccgtgttcct

SEQ ID NO: 42 - DNA, 24 bp, Synthetic construct
  source 1..24: mol_type = other DNA
tgatgtcatcatacttggcaggtt

SEQ ID NO: 43 - DNA, 22 bp, Synthetic construct
  source 1..22: mol_type = other DNA
atcaccccccaccagatcaagc

SEQ ID NO: 44 - DNA, 23 bp, Synthetic construct
  source 1..23: mol_type = other DNA
gtccttgtcgtacaggccgttca

(19) United States
(12) Patent Application Publication
(10) Pub. No.: US 2024/0408243 A1
(43) Pub. Date: Dec. 12, 2024

(54) METHODS FOR UPREGULATING SHANK3 EXPRESSION

(71) Applicant: Massachusetts Institute of Technology, Cambridge, MA (US)

(72) Inventors: Guoping Feng, Cambridge, MA (US); Qian Chen, Cambridge, MA (US)

(73) Assignee: Massachusetts Institute of Technology, Cambridge, MA (US)

(21) Appl. No.: 18/812,490

(22) Filed: Aug. 22, 2024

Related U.S. Application Data
(63) Continuation of application No. PCT/US2023/063123, filed on Feb. 23, 2023.
(60) Provisional application No. 63/313,250, filed on Feb. 23, 2022.

(51) Int. Cl. A61K 48/00; A61K 9/00; A61K 38/17; A61P 25/00; C12N 9/22; C12N 15/11; C12N 15/86

(52) U.S. Cl. CPC A61K 48/0066; A61K 9/0085; A61K 38/1709; A61K 48/0075; A61P 25/00; C12N 9/22; C12N 15/111; C12N 15/86; C12N 2310/20; C12N 2750/14143

(57) ABSTRACT

Aspects of the disclosure relate to non-naturally occurring constructs for the activation of Shank3 gene expression, AAV vectors comprising the constructs, and gene therapy methods.

### RELATED APPLICATION

This application claims the benefit under 35 U.S.C. § 119 (e) of U.S. Provisional Application No. 63/313,250, filed on Feb. 23, 2022, entitled “METHODS FOR UPREGULATING SHANK3 EXPRESSION,” the entire contents of which are incorporated herein by reference.

### REFERENCE TO AN ELECTRONIC SEQUENCE LISTING

The contents of the electronic sequence listing (B119570150WO00-SEQ-OMJ.xml; Size: 139,550 bytes; and Date of Creation: Feb. 21, 2023) is herein incorporated by reference in its entirety.

### FIELD OF THE INVENTION

The present disclosure relates to the use of CRISPR-mediated activation for upregulating Shank3 gene expression.

### BACKGROUND

Neurodevelopmental disorders such as autism spectrum disorders (ASD) and intellectual disability (ID) are some of the most debilitating brain disorders. ASD and ID, similar to most monogenic disorders, commonly only have one of the two copies of the gene disrupted. Deletions and/or mutations involving Shank3 account for about 0.5-1% of all ASD patients and about 2% of ASD patients with ID. However, there is no effective treatment for ASD and/or ID. Several challenges have arisen to developing pharmacological treatments that could correct the multitude of pathologies associated with ASD and ID.

### SUMMARY

Aspects of the disclosure relate to the use of CRISPR-mediated activation for upregulating Shank3 expression.

Aspects of the disclosure relate to constructs comprising a) a Shank3 guide RNA (gRNA) that hybridizes to a nucleic acid sequence of a Shank3 gene; b) a gene editing protein or fragment thereof; and c) a transcriptional activator. In some embodiments, the gene editing protein or fragment thereof is a protein of the CRISPR/Cas9 system

In some embodiments, the protein of the CRISPR/Cas9 system comprises a nuclease-dead CRISPR/Cas9 protein. In some embodiments, the nuclease-dead CRISPR/Cas9 protein is a S. aureus dCas9 protein (dSaCas9).

In some embodiments, the transcriptional activator is VP64.

In some embodiments, the Shank3 gRNA hybridizes to a region from about +139 to +159 relative to the transcriptional start site of the Shank3 gene. In some embodiments, the Shank3gRNA hybridizes to a region from about −64 to −84 relative to the transcriptional start site of the Shank3 gene. In some embodiments, the Shank3 gRNA hybridizes to a region from about −124 to −104 relative to the transcriptional start site of the Shank3 gene. In some embodiments, the Shank3 gRNA hybridizes to a region from about −190 to −170 relative to the transcriptional start site of the Shank3 gene. In some embodiments, the Shank3 gRNA hybridizes to a region from about −228 to −208 relative to the transcriptional start site of the Shank3 gene. In some embodiments, the Shank3 gRNA hybridizes to a region from about-303 to −283 relative to the transcriptional start site of the Shank3 gene. In some embodiments, the Shank3 gRNA hybridizes to a region from about −359 to −339 relative to the transcriptional start site of the construct.

In some embodiments, the Shank3 gRNA sequence comprises a nucleic acid sequence of any one of SEQ ID NOs: 1-7. In some embodiments, the Shank3 gRNA sequence comprises SEQ ID NO: 3.

In some embodiments, the construct further comprising at least one promoter. In some embodiments, the at least one promoter is a CMV promoter, a U6 promoter, and/or a human Synapsin 1 (hSyn1) promoter.

In some embodiments, the construct comprises a first promoter that drives expression of the gRNA and a second promoter that drives expression of the protein of the CRISPR/Cas9 system. In some embodiments, the first promoter is the U6 promoter and the second promoter is the CMV promoter. In some embodiments, the first promoter is the U6 promoter and the second promoter is the hSyn 1 promoter.

In some embodiments, the construct further comprises adeno-associated virus (AAV) inverted terminal repeats (ITRs). In some embodiments, the ITRs are adeno-associated virus ITRs of a serotype selected from the group consisting of AAVI ITR, AAV2 ITR, AAV3 ITR. AAV4 ITR, AAVS ITR, AAV6 ITR, and AAV9 ITR. In some embodiments, the ITRs are AAV2 and/or AAV9 ITRs. In some embodiments, the ITR comprises a sequence that is at least 90% identical to SEQ ID NO: 29 or 30. In some embodiments, the ITR comprises the sequence of SEQ ID NO: 29 or 30.

In some embodiments, the construct further comprises a polyadenylation signal positioned between the dSaCas9 and the 3′ ITR. In some embodiments, the polyadenylation signal is a bovine growth hormone (bGH) polyadenylation signal.

In some embodiments, the construct is capable of upregulating Shank3 gene expression in vivo. In some embodiments, the construct is capable of upregulating Shank3 gene expression at least to the level of Shank3 gene expression in a control subject. In some embodiments, the control subject does not have disruption of the Shank3 gene.

In some embodiments, the construct comprises a sequence that is at least 90% identical to SEQ ID NO: 17. In some embodiments, the construct comprises the sequence of SEQ ID NO: 17.

Further aspects of the disclosure relate to vectors comprising constructs described herein. In some embodiments, the vector is a viral vector. In some embodiments, the viral vector is an AAV vector.

Further aspects of the disclosure relate to recombinant adeno-associated viruses (rAAVs) comprising constructs described herein and at least one AAV capsid protein. In some embodiments, the capsid protein is of a serotype selected from AAV1, AAV2, AAV3, AAV4, AAV5, AAV6, AAV6.2, AAV7, AAV8, AAVrh.8, AAV9, AAVrh.10, AAVrh39, and AAVrh.43. In some embodiments, the serotype is AAV2. In some embodiments, the serotype is AAV9.

Further aspects of the disclosure relate to methods of activating Shank3 gene expression in a subject in need thereof by administering constructs described herein. Further aspects of the disclosure relate to methods of upregulating Shank3 gene expression by administering vectors or rAAVs described herein. Further aspects of the disclosure relate to methods of administering vectors or rAAVs described herein.

In some embodiments, the vector or rAAV is administered intracerebroventricularly. In some embodiments, the vector or rAAV is delivered to the brain of the subject. In some embodiments, the vector or rAAV is delivered to the cortex, striatum, cerebellum, brain stem, and/or thalamus of the subject. In some embodiments, the vector or rAAV is administered intravenously.

In some embodiments, the subject is a human subject. In some embodiments, the human subject is an adult. In some embodiments, the human subject is not an adult. In some embodiments, the human subject is a newborn. In some embodiments, the human subject is a child older than 1 year old.

In some embodiments, the subject has or is suspected of having, or is at risk of having, a neurodevelopmental disorder. In some embodiments, the subject has, is suspected of having, or is at risk of having, an autism spectrum disorder (ASD). In some embodiments, the subject exhibits one or more symptoms of an ASD. In some embodiments, the subject has, is suspected of having, or is at risk of having, Phelan-McDermid syndrome. In some embodiments, the subject exhibits one or more of: developmental delay, intellectual disability (ID), sleep disturbance, hypotonia, lack of speech, or language delay. In some embodiments, the subject has or is suspected of having, or is at risk of having, reduced expression of the Shank3 gene relative to a control subject. In some embodiments, the control subject is a subject that does not have or is not suspected of having, or is not at risk of having, a neurodevelopmental disorder, an autism spectrum disorder (ASD), and/or Phelan-McDermid syndrome.

In some embodiments, reduced expression of the Shank3 gene is caused by disruption of at least one copy of the Shank3 gene. In some embodiments, disruption of the Shank3 gene comprises a deletion or inactivation in at least one copy of the Shank3 gene. In some embodiments, disruption of the Shank3 gene comprises one or more mutations within at least one copy of the Shank3 gene.

Further aspects of the present disclosure relate to methods for upregulating expression of Shank3 in a subject in need thereof.

Further aspects of the present disclosure relate to methods of treating a subject having a neurodevelopmental disorder. In some embodiments, the disclosure relates to methods of treating a subject having an autism spectrum disorder (ASD). In some embodiments, the disclosure relates to methods of treating a subject having Phelan-McDermid syndrome. In some embodiments, the methods of treatment comprise administering to the subject an effective amount of a composition comprising an AAV vector that comprises a construct comprising a) a Shank3 guide RNA (gRNA) that hybridizes to a nucleic acid sequence of a Shank3 gene, b) a gene editing protein or fragment thereof, wherein the gene editing protein or fragment thereof is a protein of the CRISPR/Cas9 system, and c) a transcriptional activator described herein.

In some embodiments, the subject has improved sleep efficiency after being administered to an effective amount of the composition described herein. In some embodiments, the composition includes a pharmaceutically acceptable carrier.

### BRIEF DESCRIPTION OF THE DRAWINGS

The accompanying drawings are not intended to be drawn to scale. The drawings are illustrative only and are not required for enablement of the disclosure. For purposes of clarity, not every component may be labeled in every drawing. In the drawings:

FIGS. 1A-1E show the design and optimization of a CRISPR/dSaCas9 transcription activator system for the Shank3 gene. FIG. 1A shows a schema of upregulation of the normal copy of the Shank3 gene to compensate for the loss of the disrupted copy for gene therapy of Shank3 haploinsufficiency. FIG. 1B shows a schema illustrating the gene transcription activation system mediated by CRISPR-mediated activation (CRISPRa). CMV: cytomegalovirus promoter; SV40 NLS: a nuclear localization signal from simian virus 40; dSaCas9: nuclease-dead Staphylococcus aureus Cas9; VP64: a transcription activator composed of four tandem copies of VP16 (Herpes Simplex Viral Protein 16); bGH polyA: the bovine growth hormone polyadenylation signal; and ITR: inverted terminal repeats. FIG. 1C shows the genomic locus of Shank3 gRNAs. TSS: transcriptional start site. FIG. 1D shows a differential interference contrast (DIC, left) and an enhanced green fluorescent protein (EGFP, right) fluorescent image of Neruo-2a cells transfected with an elongation factor 1 (EF1) alpha promoter-driven EGFP construct. Scale bar: 50 μm. FIG. 1E shows results of CRISPRa comprising Shank3 gRNAs (Shank3 gRNAs 1-7) in Neuro-2a cells targeting the Shank3 promoter. Results are expressed as Shank3 mRNA fold-increase normalized to Gapdh using the ΔΔCt method. N=5 in each group. The data are represented as means±SEM from 2 independent experiments. **p-value<0.01 ***p-value<0.0001 (one-way ANOVA, Tukey test).

FIGS. 2A-2G show that the delivery of Shank3 CRISPRa rAAV to mouse brains upregulated Shank3 in Shank3-InG3680^{+/-} mice. FIG. 2A shows a schema of intracerebroventricular injection (ICV) of AAVs. FIG. 2B shows post neonatal day 1 (P1) mice after ICV-injection. Inset: The lateral ventricles are shown in ICV-injected pups with AAVs mixed with fast-green dye. FIG. 2C shows an EGFP fluorescent image of a mouse ICV-injected with AAV2/9-nls-EGFP at post neonatal day 30 (P30). FIG. 2D shows that CRISPRa in mouse brain targeting the Shank3 promoter significantly increased Shank3 mRNA levels, but not Shank1 (FIG. 2E) or Shank2 (FIG. 2F) levels. Scale bar: 1 mm. Results are expressed as mRNA fold-increase normalized to Gapdh using the ΔΔCt method. The data are represented as means±SEM. * p-value<0.05, **p-value<0.01. ns: no significant difference (n=3 in each group, one-way ANOVA, Tukey test). FIG. 2G provides an immunoblot that shows that the major isoform of Shank3 protein (Shank3a) was induced by the dSaCas9/sgRNA treatment (n=3 in each group).

FIGS. 3A-31 show that Shank3 CRISPRa rAAV upregulated Shank3 in 3 to 4-month-old Shank3-InG3680^{+/-} mice. FIG. 3A shows that control CRISPRa rAAV could not increase Shank3 gene expression in both WT and Shank3-InG3680^{+/-} mice. The data are represented as means±SEM (n=4 in WT injected with PBS, n=4 in WT injected with control rAAVs, n=6 in Shank3-InG3680^{+/-} injected with PBS and n=7 in Shank3-InG3680^{+/-} injected with control rAAVs). FIG. 3B shows that CRISPRa rAAV in mouse brain targeting the Shank3 promoter significantly increased Shank3 mRNA levels in 3 to 4-month-old Shank3-InG3680^{+/-} mice. The data are represented as means±SEM (n=8 in WT, n=14 in Shank3-InG3680^{+/-}; n=7 in Shank3-InG3680^{+/-} injected with rAAV containing gRNA2-Shank3 CRISPRa, n=5 in Shank3-InG3680^{+/-} injected with rAAV containing gRNA3-Shank3 CRISPRa, n=7 in Shank3-InG3680^{+/-} injected with rAAV containing gRNA6-Shank3 CRISPRa and n=6 in Shank3-InG3680^{+/-} injected with rAAV containing gRNA7-Shank3 CRISPRa). * p-value<0.05 and ***p-value<0.005 (One-way ANOVA, Tukey test, compared to Shank3-InG3680^{+/-} group). FIG. 3C provides an immunoblot showing that the major isoform of Shank3 protein (Shank3α) in 3 to 4-month-old Shank3-InG3680^{+/-} mice was increased by the Shank3 CRISPRa rAAV injection. Quantification results of immunoblot are shown as Shank3a normalized protein expression (FIG. 3D), Shank3c/d normalized protein expression (FIG. 3E) and Shank3e normalized expression (FIG. 3F) of WT and Shank3-InG3680^{+/-} mice injected with PBS or control rAAVs. The data are represented as means±SEM (ns, no signification difference, n=3 in WT injected with PBS, n=4 in WT injected with control rAAVs, n=5 in Shank3-InG3680^{+/-} injected with PBS and n=8 in Shank3-InG3680^{+/-} injected with control rAAVs). Normalized Shank3a protein expression (FIG. 3G), normalized Shank3c/d protein expression (FIG. 3H) and normalized Shank3e protein expression (FIG. 3I) of WT, Shank3-InG3680^{+/-} mice and Shank3-InG3680^{+/-} mice injected with Shank3-CRISPRa rAAVs. The data are represented as means±SEM (n=3 in WT, n=8 in Shank3-InG3680^{+/-}, n=3 in Shank3-InG3680^{+/-} injected with rAAV containing gRNA2-Shank3 CRISPRa, n=3 in Shank3-InG3680^{+/-} injected with rAAV containing gRNA3-Shank3 CRISPRa, n=3 in Shank3-InG3680^{+/-} injected with rAAV containing gRNA6-Shank3 CRISPRa and n=3 in Shank3-InG3680^{+/-} injected with rAAV containing gRNA7-Shank3 CRISPRa). * p-value<0.05 and **p-value<0.01 (One-way ANOVA, Tukey test, compared to Shank3-InG3680^{+/-} group).

FIGS. 4A-4E show immunostaining of Shank3, which demonstrated that CRISPRa could upregulate Shank3 in different brain regions. FIG. 4A shows immunofluorescence images from WT, Shank3-InG3680^{+/-} and Shank3-InG3680^{+/-} mice. Top panels: Immunofluorescence images of sagittal section from WT, Shank3-InG3680^{+/-} and Shank3-InG3680^{+/-} mice injected with gRNA3-containing CRISPRa rAAV virus. Bottom panels: the zoom-in immunofluorescence images of striatum and hippocampus from the top panel images. Scale bar: 1 mm in top panels and 500 μm in bottom panels. FIGS. 4B-4E show quantification results of immunofluorescence intensity of striatum (FIG. 4B), hippocampus (FIG. 4C), cortex (FIG. 4D) and thalamus (FIG. 4E) from different experimental groups. The data are represented as means±SEM (n=5 in WT injected with PBS, n=4 in WT injected with control rAAVs, n=5 in Shank3-InG3680^{+/-} injected with PBS, n=8 in Shank3 InG3680^{+/-} injected with control rAAVs, n=7 in Shank3-InG3680^{+/-} injected with rAAV containing gRNA2-Shank3 CRISPRa, n=5 in Shank3-InG3680^{+/-} injected with rAAV containing gRNA3-Shank3 CRISPRa, n=7 in Shank3-InG3680^{+/-} injected with rAAV containing gRNA6-Shank3 CRISPRa and n=6 in Shank7-InG3680^{+/-} injected with rAAV containing gRNA3-Shank3 CRISPRa). **p-value<0.01, ***p-value<0.005 and ****p-value<0.0001 (One-way ANOVA, Tukey test, compared to Shank3-InG3680^{+/-} injected with PBS group).

FIGS. 5A-5H show CRISPRa Shank3 gene induction in mouse brains under the control of the neuronal specific human Synapsin I promoter. FIG. 5A provides a schema showing swapping the CMV promoter for the human Synapsin I (hSyn) promoter in a gene transcription activation system mediated by CRISPRa. FIG. 5B shows that CRISPRa with the hSyn promoter in mouse brain targeting the Shank3 promoter significantly increased Shank3 mRNA levels. Results are expressed as Shank3 relative mRNA fold-increase normalized to Gapdh using the ΔΔCt method. The data are represented as means±SEM (n=18 in WT, n=19 in Shank3 InG3680^{+/-} and n=8 in Shank3-InG3680^{+/-} injected with rAAV containing gRNA3-Shank3 CRISPRa). **p-value<0.01 and ***p-value<0.005

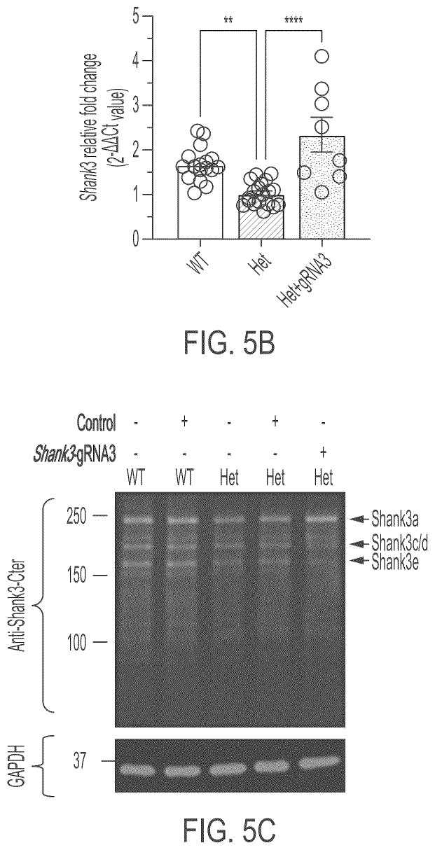

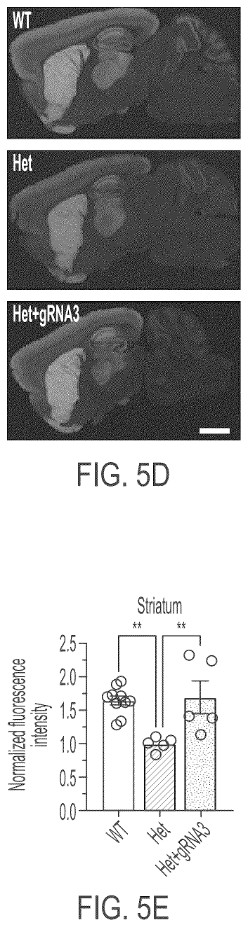

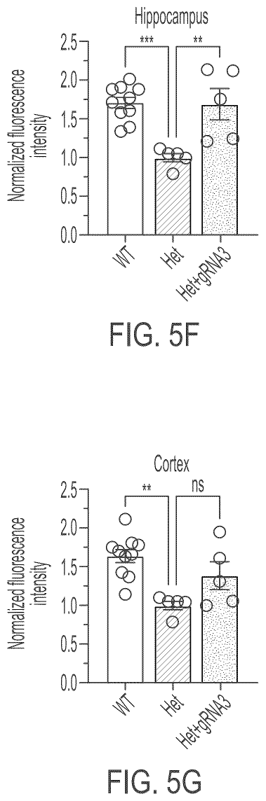

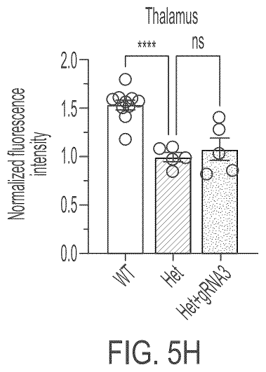

(One-way ANOVA, Tukey test, compared to Shank3-InG3680^{+/-} group). FIG. 5C provides an immunoblot showing that the major isoform of Shank3 protein (Shank3α) was induced by the dSaCas9/sgRNA3 treatment. FIG. 5D shows immunofluorescence images of sagittal section from WT, Shank3-InG3680^{+/-} and Shank3-InG3680^{+/-} mice injected with gRNA3-containing CRISPRa AAV virus. Quantification results of immunofluorescence intensity of striatum (FIG. 5E), hippocampus (FIG. 5F), cortex (FIG. 5G) and thalamus (FIG. 5H) from different experimental groups (WT, a Shank3-InG3680^{+/-} and a Shank3-InG3680^{+/-} injected with gRNA3-containing CRISPRa AAV virus) are shown. The data are represented as means±SEM (n=10 in WT, n=5 in Shank3-InG3680^{+/-} and n=5 in Shank3-InG3680^{+/-} injected with rAAV containing gRNA3-Shank3 CRISPRa). **p-value<0.01, ***p-value<0.005 and ****p-value<0.0001 (One-way ANOVA, Tukey test, compared to Shank3-InG3680^{+/-} group).

### DETAILED DESCRIPTION

Genomic alterations resulting in reduced activity of one or more genes or gene products may be a causative factor in a myriad of mammalian diseases. One such genomic alteration is haploinsufficiency, in which there is only one functional copy of a gene and that single copy does not produce enough of the gene product to produce a wild-type phenotype.

Aspects of the disclosure relate to gene therapy approaches involving CRISPR-mediated activation for treating neurodevelopmental disorders caused by Shank3 haploinsufficiency. The Examples surprisingly demonstrate that increased transcription of Shank3 can be achieved with a transcription-activating guide-RNA (gRNA) construct (e.g., as part of a dCas9/gRNA complex) targeted to a promoter region of Shank3. Moreover, the Examples demonstrate that transcriptional activation of Shank3 upregulates Shank3 expression to wild-type levels. This system can be administered to subjects to treat diseases using gene therapy. Gene therapy strategies disclosed herein can use, e.g., recombinant adeno-associated virus (rAAV) to deliver a CRISPR-mediated activation system to upregulate the Shank3 gene in brain cells and to restore cellular function.

### Shank3

The Shank family of proteins (e.g., SHANK1, SHANK2, and SHANK3) are master scaffolding proteins that tether and organize proteins at the synapses of excitatory neurons. Members of this family share at least five main domain regions: N-terminal ankyrin repeats, SH3 domain, PDZ domain, proline-rich region, and a C-terminal SAM domain. Through these functional domains, Shank proteins interact with many postsynaptic density (PSD) proteins. SHANK3 is one of three members of the SHANK proteins that organizes a cytoskeleton-associated signaling complex at postsynaptic density (PSD) as a scaffolding protein and interacts with various synaptic molecules including GluR1 of a-amino-3-hydroxy-5-methyl-4-isoxazole propionic acid receptor (AMPAR), N-methyl-D-aspartate receptor (NMDAR) via the postsynaptic density-95 (PSD-95)/guanylate kinase-associated protein (GKAP) complex.

Deletion and/or mutation of Shank3 is a major cause of the core neurodevelopmental and neurobehavioral deficits in Phelan-McDermid syndrome with a high prevalence of autism spectrum disorder (ASD). Human genetic studies have identified Shank3 mutations as accounting for about 1% of ASD cases. Patients with Phelan-McDermid syndrome and other individuals with Shank3 mutations often exhibit a variety of comorbid traits, which include developmental delay, sleep disturbances, hypotonia, lack of speech or severe language delay, and characteristic features of ASD. Currently, there is no effective treatment for ASD.

The association of ASD with Shank3 provided an immediate link between synaptic dysfunction and the pathophysiology of ASD. Animal models bridge the human genetics of ASD to brain pathology underlying clinical presentation, and ultimately help to discover and evaluate effective therapeutics. Previous studies in flies, fish, and rodents have revealed synaptic dysfunction and behavioral abnormalities due to loss of Shank3. For example, disruption of Shank3 in mouse models has resulted in synaptic defects, impaired social interactions, motor difficulties, repetitive grooming and increased anxiety levels. Furthermore, Shank3-deficient mouse models present predictive validity as the synaptic defects and behavioral abnormalities are reversible when Shank3 is restored.

Like other monogenic disorders, in disorders associated with Shank3 disruption, only one of the two copies of the gene is disrupted. Based on results described herein, upregulation of the expression of the normal copy of the Shank3 gene to compensate for the loss of the disrupted copy, through transcriptional activation, is a promising gene therapy approach to treating disorders associated with disruption of the Shank3 gene.

### Upregulation of Shank3 Using CRISPR-Mediated Activation

The CRISPR/Cas genome-editing system makes it possible to achieve gene therapy with precision. The CRISPR/Cas system uses a short RNA sequence that matches the genomic region of interest to precisely guide genome-editing capability to that gene. The term “CRISPR” refers to “clustered regularly interspaced short palindromic repeats”, which are DNA loci containing short repetitions of base sequences. CRISPR loci form a portion of a prokaryotic adaptive immune system that confers resistance to foreign genetic material. In the Type II CRISPR system, a spacer DNA hybridizes to transactivating RNA (tracrRNA) and is processed into CRISPR-RNA (crRNA) and subsequently associates with CRISPR-associated nucleases (Cas nucleases) to form complexes that recognize and degrade foreign DNA. Examples of CRISPR nucleases include, but are not limited to Cas9, dCas9, Cas6, Cpf1, and variants thereof.

The CRISPR/Cas system can perform different types of genetic modifications, including the activation of genes. For example, the CRISPR/Cas system can activate genes using modified versions of CRISPR effectors that do not have endonuclease activity, fused to a transcriptional activator to increase expression of a gene of interest. This approach is referred to interchangeably herein as CRISPR activation or CRISPR-mediated activation (“CRISPRa”). In some embodiments, the CRISPR effector is a nuclease that has reduced or eliminated nuclease activity. In some embodiments, the CRISPR effector is a Cas9 that has reduced or eliminated nuclease activity. In some embodiments, the CRISPR effector is a nuclease-dead Cas9 protein (dCas9). The nuclease that has reduced or eliminated nuclease activity (e.g., dCas9) can be fused with a transcriptional activator, a transcription effector protein, or a transcription coactivator to form a chimeric protein.

The present disclosure provides constructs that are suitable to be delivered in vivo or in vitro for activating Shank3 gene expression. Constructs described herein can activate endogenous Shank3 gene expression from the normal (e.g., not disrupted) copy of the endogenous gene to compensate for the loss of the disrupted copy due to Shank3 haploinsufficiency. Accordingly, constructs provided herein are suitable for treating or improving neurodevelopmental disorders that are caused by disrupted Shank3 gene expression.

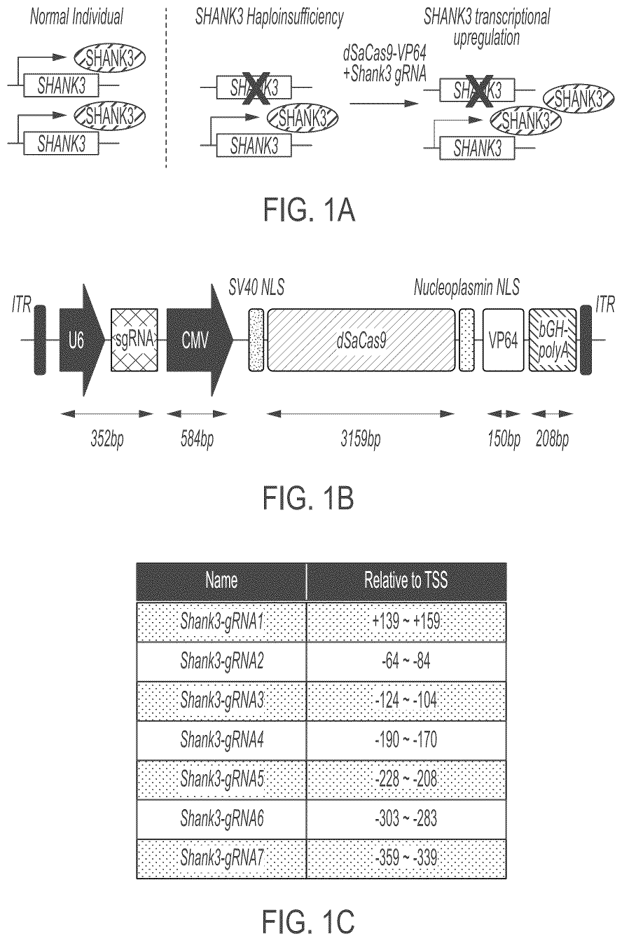

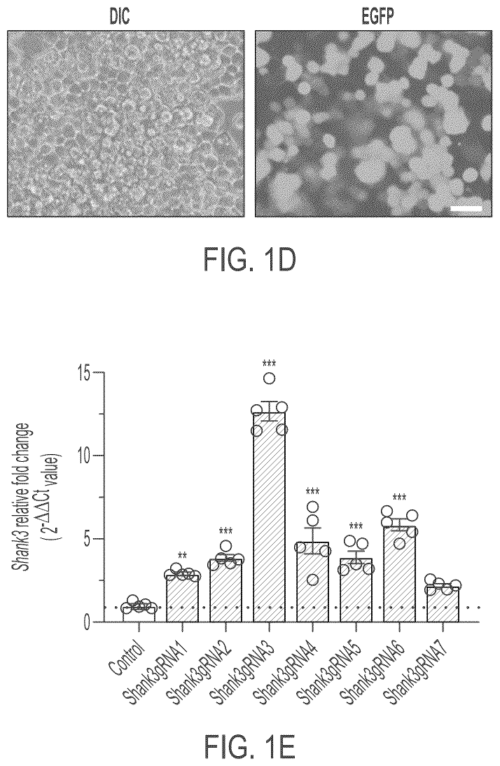

As shown in FIG. 1B, in some embodiments, the present disclosure provides constructs that comprise: a) a Shank3 guide RNA (gRNA) that hybridizes to a nucleic acid sequence of a Shank3 gene; b) a gene editing protein or fragment thereof; and c) a transcription activator.

The present disclosure provides novel Shank3 guide RNAs (gRNAs) suitable for targeting the Shank3 gene. gRNAs are RNA molecules that function as guides for RNA- or DNA-targeting enzymes. gRNAs can be naturally occurring RNAs identified in cells and tissues or can be designed and synthesized. For example, gRNAs can be designed for gene editing techniques such as CRISPR/Cas9 genome editing. gRNAs can be chemically modified.

Constructs described herein include Shank3 gRNAs. As used herein, “a Shank3 gRNA” refers to a gRNA that hybridizes to a nucleic acid sequence of a Shank3 gene.

In some embodiments, a gRNA is between 1 and 30 nucleotides in length. In some embodiments, a gRNA is between 5 and 25 nucleotides in length. In some embodiments, a gRNA is between 10 and 22 nucleotides in length. In some embodiments, a gRNA is between 14 and 24 nucleotides in length. For example, in some embodiments, a gRNA is 14, 15, 16, 17, 18, 19, 20, 21, 22, 23 24, 25, 26, 27, 28, 29, or 30 nucleotides in length.

In some embodiments, a Shank3 gRNA hybridizes at least in part to a regulatory region of a Shank3 gene. In some embodiments, a Shank3 gRNA hybridizes at least in part to a promoter of a Shank3 gene. In some embodiments, a Shank3 gRNA hybridizes at least in part to an open reading frame of a Shank3 gene. As shown in FIG. 1C, a Shank3 gRNA can be described based on where it hybridizes relative to the transcriptional start site (“TSS”) of the Shank3 gene. As shown in the Examples, the relative position of the Shank3 gRNAs to the Shank3 TSS was found to affect the capability of the Shank3 gRNA for upregulating endogenous Shank3 gene expression.

In some embodiments, a Shank3 gRNA hybridizes to a region from-400 to +200 relative to the Shank3 TSS. For example, in some embodiments, a Shank3 gRNA can hybridize to a contiguous stretch of nucleotides that includes any one of nucleotides-400, −399, −398, −397, −396, −395, −394, −393, −392, −391, −390, −389, −388, −387, −386, −385, −384, −383, −382, −381, −380, −379, −378, −377, −376, −375, −374, −373, −372, −371, −370, −369, −368, −367, −366, −365, −364, −363, −362, −361, −360, −359, −358, −357, −356, −355, −354, −353, −352, −351, −350, −349, −348, −347, −346, −345, −344, −343, −342, −341, −340, −339, −338, −337, −336, −335, −334, −333, −332, −331, −330, −329, −328, −327, −326, −325, −324, −323, −322, −321, −320, −319, −318, −317, −316, −315, −314, −313, −312, −311, −310, −309, −308, −307, −306, −305, −304, −303, −302, −301, −300, −299, −298, −297, −296, −295, −294, −293, −292, −291, −290, −289, −288, −287, −286, −285, −284, −283, −282, −281, −280, −279, −278, −277, −276, −275, −274, −273, −272, −271, −270, −269, −268, −267, −266, −265, −264, −263, −262, −261, −260, −259, −258, −257, −256, −255, −254, −253, −252, −251, −250, −249, −248, −247, −246, −245, −244, −243, −242, −241, −240, −239, −238, −237, −236, −235, −234, −233, −232, −231, −230, −229, −228, −227, −226, −225, −224, −223, −222, −221, −220, −219, −218, −217, −216, −215, −214, −213, −212, −211, −210, −209, −208, −207, −206, −205, −204, −203, −202, −201, −200, −199, −198, −197, −196, −195, −194, −193, −192, −191, −190, −189, −188, −187, −186, −185, −184, −183, −182, −181, −180, −179, −178, −177, −176, −175, −174, −173, −172, −171, −170, −169, −168, −167, −166, −165, −164, −163, −162, −161, −160, −159, −158, −157, −156, −155, −154, −153, −152, −151, −150, −149, −148, −147, −146, −145, −144, −143, −142, −141, −140, −139, −138, −137, −136, −135, −134, −133, −132, −131, −130, −129, −128, −127, −126, −125, −124, −123, −122, −121, −120, −119, −118, −117, −116, −115, −114, −113, −112, −111, −110, −109, −108, −107, −106, −105, −104, −103, −102, −101, −100, −99, −98, −97, −96, −95, −94, −93, −92, −91, −90, −89, −88, −87, −86, −85, −84, −83, −82, −81, −80, −79, −78, −77, −76, −75, −74, −73, −72, −71, −70, −69, −68, −67, −66, −65, −64, −63, −62, −61, −60, −59, −58, −57, −56, −55, −54, −53, −52, −51, −50, −49, −48, −47, −46, −45, −44, −43, −42, −41, −40, −39, −38, −37, −36, −35, −34, −33, −32, −31, −30, −29, −28, −27, −26, −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109. 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 and 200 relative to the Shank3 TSS.

In some embodiments, the Shank3 gRNA hybridizes to a region from about +139 to +159 relative to the TSS of the Shank3 gene. In some embodiments, the Shank3 gRNA hybridizes to a region from about −64 to −84 relative to the TSS of the Shank3 gene. In some embodiments, the Shank3 gRNA hybridizes to a region from about −124 to −104 relative to the TSS of the Shank3 gene. In some embodiments, the Shank3 gRNA hybridizes to a region from about −190 to −170 relative to the TSS of the Shank3 gene. In some embodiments, the Shank3 gRNA hybridizes to a region from about −228 to −208 relative to the TSS of the Shank3 gene. In some embodiments, the Shank3 gRNA hybridizes to a region from about-303 to −283 relative to the TSS of the Shank3 gene. In some embodiments, the Shank3 gRNA hybridizes to a region from about −359 to −339 relative to the TSS of the Shank3 gene.

In some embodiments, the Shank3 gRNA comprises a nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical of any one of SEQ ID NOs: 1-7. In some embodiments, the Shank3 gRNA comprises the nucleic acid sequence of any one of SEQ ID NOs: 1-7. In some embodiments, the Shank3 gRNA sequence comprises SEQ ID NO: 3. In some embodiments, the Shank3 gRNA comprises a nucleic acid sequence that differences from the sequence of any one of SEQ ID NOs: 1-7 by no more than 1, 2, 3, 4, 5, 6, 7, 8, 9, or 10 nucleotides.

In some embodiments gRNAs further include a scaffold region. In some embodiments, the scaffold region comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 21. In some embodiments, the scaffold region comprises a sequence of SEQ ID NO: 21.

In some embodiments, one or more nucleotides of the Shank3 gRNA can be chemically modified. Chemical modifications may involve, e.g., sugar modifications (e.g., locked nucleic acid), sugar/backbone modifications (e.g., mirror DNA), backbone modifications (e.g., phosphorothioate), base modifications (e.g., C5-modified uridine), or unnatural base pairs. For example, one or more nucleotides of the Shank3 gRNA may be chemically modified at the 2′-position (e.g., 2′-O-methyl, 2′-O-methoxyethyl (2′-MOE), and 2′-fluoro) or the 4′-position (e.g., 4′-thiol). In some embodiments, mutations (e.g., substitutions) may be made in a gRNA nucleic acid sequence by various methods known to one of ordinary skill in the art, such as, e.g., PCR-directed mutagenesis or any other method suitable for aspects of the disclosure.

In some embodiments, the gene editing protein or fragment thereof included within a construct described herein is a protein of the CRISPR/Cas system. In some embodiments, the protein of the CRISPR/Cas system comprises a nuclease-dead CRISPR/Cas protein. In some embodiments, the nuclease-dead CRISPR/Cas protein is a nuclease-dead Cas9 protein. As used herein, “dCas9” refers to dead Cas9 or Cas9 Endonuclease Dead, which is a mutant form of Cas9 which lacks endonuclease activity due to point mutations in its endonuclease domains. For example, point mutations (e.g., D10A and H840A) can be introduced into each nucleolytic domain, RuvC and HNH, of a wild type Cas9 protein to form a dCas9. It should be appreciated that any nuclease-dead CRISPR/Cas protein may be compatible with aspects of the disclosure.

In some embodiments, the nuclease-dead Cas9 protein is a Staphylococcus. aureus (S. aureus) dCas9 protein (dSaCas9). In some embodiments, the dCas9 comprises a nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 23. In some embodiments, the dCas9 comprises the nucleic acid sequence of SEQ ID NO: 23. In some embodiments, the dCas9 comprises a protein sequence encoded by SEQ ID NO: 23 that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 24. In some embodiments, the dCas9 comprises the protein sequence of SEQ ID NO: 24. It should be appreciated that a nuclease-dead Cas9 protein can be derived from any source, such as any prokaryotic organism that may be suitable for aspects of the disclosure. For example, in some embodiments, the nuclease-dead Cas9 protein can by derived from Streptococcus pyogenes (S. pyogenes). In some embodiments, the nuclease-dead CRISPR/Cas protein may be dCasX (e.g., DpbCas12e protein from Deltaproteobacteria), dNmCas9 (e.g., derived from N. meningiditis), dSauriCas9 (e.g., derived from Staphylococcus Auricularis), dCjCas9 (e.g., derived from Campylobacter jejuni), dFnCas9 (e.g., derived from Francisella novicida), dSt1Cas9 (e.g., derived from Streptococcus thermophilus), dSt3Cas9 (e.g., derived from Streptococcus thermophilus), dAsCPf1 (e.g., derived from Acidaminococcus), or dLbCpf1 (e.g., derived from Lachnospiraceae bacterium ND2006).

In some embodiments, the gene editing protein or fragment thereof is a Cas6 protein or variants thereof. In some embodiments, the gene editing protein or fragment thereof is a nuclease dead Cas6 protein or fragment thereof.

Constructs described herein include transcriptional activators. In some embodiments, the transcriptional activator is VP64. VP64 includes four tandem copies (or tandem repeats) of VP16 (a herpes simplex virus type 1 transcription factor), which regulates viral gene transcription. In some embodiments, the transcriptional activator can be any multimer of VP16 that is suitable for the present disclosure, including, e.g., VP48, VP160, or VP192. Non-limiting examples of transcriptional activators, transcription effector proteins, or transcription coactivators include VP(16)ₙ, p300, the Epstein-Barr virus R trans-activator (e.g., RTA), p65 (mouse or human), SunTag (e.g., a repeating peptide array), General Control Nondepressible (e.g., GCN4), or p65 with heat shock factor 1 (HSF1).

In some embodiments, the transcriptional activator comprises a nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 26. In some embodiments, the transcriptional activator comprises the nucleic acid sequence of SEQ ID NO: 26. In some embodiments, the transcriptional activator comprises a protein sequence encoded by SEQ ID NO: 26 that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 27. In some embodiments, the transcriptional activator comprises the protein sequence of SEQ ID NO: 27. It should be appreciated that transcriptional activators compatible with aspects of the disclosure can include any transcription factor known in the art. Without wishing to be bound by any theory, transcription factors may comprise a DNA-binding domain (DBD), an activation domain (AD), and/or a signal-sensing domain (SSD). In some embodiments, a transcriptional activator that is relatively small in size, such as VP64, is preferred.

In some embodiments, constructs described herein may comprise additional components such as regulatory sequences or genetic regulatory elements that may facilitate activation of Shank3 gene expression. Such components may improve the stability and/or the targeting specificity of the construct.

In some embodiments, constructs described herein comprise at least one promoter. As used herein, a “promoter” refers to a DNA sequence that can be recognized by transcription machinery in a cell, which can lead to initiation of transcription of a gene. It should be appreciated that promoters in constructs described herein can be located and oriented in any way that is compatible with constructs described herein. Any promoter that is known in the art and that is compatible with aspects of the disclosure may be used.

In some embodiments, one or more promoters is a constitutive promoter. As used herein, a constitutive promoter refers to a promoter that is active in all circumstances in a cell. In some embodiments, a constitutive promoter is a CAG promoter. In some embodiments, one or more promoters is a ubiquitous promoter. As used herein, a ubiquitous promoter refers to a promoter that is expressed in most or all cell types and/or is active in initiating transcription when expressed in most or all cell types. In other embodiments, one or more promoters is primarily or only expressed in a subset of cell types and/or is active in initiating transcription only in a subset of cell types. For example, in some embodiments, one or more promoters is a promoter that is primarily or only expressed in neuronal cells and/or is primarily or only active in neuronal cells. In some embodiments, one or more promoters is a promoter that is primarily or only expressed in brain cells and/or is primarily or only active in brain cells.

In some embodiments, at least one promoter is a CMV promoter. In some embodiments, at least one promoter is a U6 promoter. In some embodiments, at least one promoter is a human Synapsin 1 (hSyn1) promoter. In some embodiments, a construct comprises a CMV promoter and a U6 promoter. In some embodiments, a construct comprises a hSyn1 promoter and a U6 promoter.

In some embodiments, one or more promoters may be a ubiquitin promoter, an EF1α promoter, an EFS promoter (EF1 alpha short promoter), a Mecp2 promoter, a CaMKIIα (calcium/calmodulin-dependent protein kinase II alpha subunit) promoter, a PGK promoter (e.g., mammalian promoter from phosphoglycerate kinase gene), a Dlx5/6 promoter, a PV promoter, or a SST (somatostatin) promoter.

In some embodiments, the construct comprises a first promoter and a second promoter. In some embodiments, the first promoter drives expression of the gRNA. In some embodiments, the second promoter drives expression of gene editing protein or fragment thereof. In some embodiments, the second promoter drives expression of a protein of the CRISPR/Cas9 system. In some embodiments, the first promoter is the U6 promoter and the second promoter is the CMV promoter. In some embodiments, the first promoter is the U6 promoter and the second promoter is the hSyn1 promoter.

In some embodiments, a promoter comprises a nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 18-20. In some embodiments, a promoter comprises a sequence that differs by no more than 1, 2, 3, 4, 5, 6, 7, 8, 9, or 10 nucleotides from the sequence of any one of SEQ ID NOs: 18-20.

In some embodiments, the construct further comprises adeno-associated virus (AAV) inverted terminal repeats (ITRs). In some embodiments, the construct is flanked by S′ and 3′ AAV ITR sequences. As known in the art, ITRs are involved in the replication and encapsidation of the AAV genome, along with its integration in the host genome and its excision. Generally, ITR sequences are about 145 bp in length. ITR sequences are known in the art, and some degree of minor modification of these sequences is permissible. The ability to modify ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)).

In some embodiments, the ITRs are adeno-associated virus ITRs of a serotype selected from the group consisting of AAVI ITR, AAV2 ITR, AAV3 ITR, AAV4 ITR, AAV5 ITR, AAV6 ITR, and AAV9 ITR. In some embodiments, the ITRs are AAV2 ITR. In some embodiments, the ITRs are AAV9 ITRs. In some embodiments, the ITRs are AAV2 ITR and AAV9 ITR. In certain embodiments, the AAV2 ITR and the AAV9 ITR can be either the 5′ ITR or the 3′ ITR in the construct. In some embodiments, the ITRs are adeno-associated virus ITRs of any serotype that may be suitable for constructs disclosed herein.

In some embodiments, the ITR comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 29 or 30. In some embodiments, the ITR comprises the sequence of SEQ ID NO: 29 or 30.

In some embodiments, the construct further comprises a polyadenylation signal positioned between the gene editing protein or fragment thereof and the 3′ ITR. In some embodiments, the polyadenylation signal is a bovine growth hormone (bGH) polyadenylation signal. It should be appreciated that any polyadenylation signal that allows for polyadenylation and thereby mediates transcription termination may be compatible with aspects of the disclosure.

In some embodiments, constructs described herein comprise a nucleic acid sequence that is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 9-17. In some embodiments, constructs described herein comprise the nucleic acid sequence of any one of SEQ ID NOs: 9-17.

In some embodiments, constructs disclosed herein are capable of upregulating Shank3 gene expression in vivo. In some embodiments, constructs disclosed herein are capable of upregulating Shank3 gene expression in vivo to wild-type levels of Shank3 gene expression. In some embodiments, constructs disclosed herein are capable of upregulating Shank3 gene expression in vivo by at least 5%, 10%, 20%, 30%, 40%, 50%, 60%, 70%, 80%, 90%, 100%, 200%, 250%, 300%, 400%, 500% or more than 500% or by at least 2-fold, at least 5-fold, at least 10-fold, at least 20-fold, at least 50-fold, at least 100-fold, or at least 1000-fold compared to a control. In some embodiments, a control may be a construct that does not contain one or more components necessary for activating Shank3 expression.

### Vectors

Vectors described herein can be used to deliver constructs comprising a) a Shank3 guide RNA (gRNA) that hybridizes to a nucleic acid sequence of a Shank3 gene; b) a gene editing protein or fragment thereof; and c) a transcriptional activator to a subject, including, e.g., delivery to specific organs or to the central nervous system (CNS) of a subject.

In some embodiments, the vector is a viral vector. In some embodiments, the vector is an AAV vector. In some embodiments, the vector is a lentiviral vector. In some embodiments, the vector is an adenovirus vector. In some embodiments, the vector is a herpes simplex virus (HSV) vector. In some embodiments, the vector comprises a construct that has a sequence that is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical, or is 100% identical, including all values in between, to any one of SEQ ID NOs: 9-17. In some embodiments, the vector comprises a construct that comprises the sequence of any one of SEQ ID NOs: 9-17.

AAV vectors described herein may be delivered to a human subject in need thereof and may be suitable for treating a human subject who has a neurodevelopmental disorder. In some embodiments, the AAV vector comprises a construct that has a sequence that is at least 90% identical to SEQ ID NO: 17. In some embodiments, the AAV vector comprises a construct that has the sequence of SEQ ID NO: 17. SEQ ID NO: 17 comprises a Shank3 gRNA having the sequence of SEQ ID NO: 3.

As disclosed herein, “identity” of sequences refers to the measurement or calculation of the percent of identical matches between two or more sequences with gap alignments addressed by a mathematical model, algorithm, or computer program that is known to one of ordinary skill in the art. The percent identity of two sequences (e.g., nucleic acid or amino acid sequences) may, for example, be determined using Basic Local Alignment Search Tool (BLAST®) such as NBLAST® and XBLAST® programs (version 2.0). Alignment technique such as Clustal Omega may be used for multiple sequence alignments. Other algorithms or alignment methods may include but are not limited to the Smith-Waterman algorithm, the Needleman-Wunsch algorithm, or Fast Optimal Global Sequence Alignment Algorithm (FOGSAA).

AAV refers to a replication-deficient Dependoparvovirus within the Parvoviridae genus of viruses. AAV can be derived from a naturally occurring virus or can be recombinant. AAV can be packaged into capsids, which can be derived from naturally occurring capsid proteins or recombinant capsid proteins. The single-stranded DNA genome of AAV includes ITRs. Without wishing to be bound by any theory, AAV vectors can comprise one or more ITRs, including a 5′ ITR and/or a 3′ ITR, one or more promoters, one or more nucleic acid sequences encoding one or more proteins of interest, and/or additional posttranscriptional regulator elements. AAV vectors disclosed herein can be prepared using standard molecular biology techniques known to one of ordinary skill in the art, as described, for example, in Sambrook el al. (Molecular Cloning: A Laboratory Manual. Cold Spring Harbor Laboratory Press, N.Y. (2012)), which is incorporated herein by reference in its entirety.

In some embodiments, an AAV vector integrates into a host cell genome. In some embodiments, an AAV vector does not integrate into a host cell genome. In some embodiments, AAV vectors can include sequences from any known organism. In some embodiments, AAV vectors can include synthetic sequences. AAV vector sequences can be modified in any way known to one of ordinary skill in the art, such as by incorporating insertions, deletions or substitutions, and/or through the use of regulatory elements, such as promoters, enhancers, and transcription and translation terminators, such as polyadenylation signals. In some embodiments, AAV vectors can include sequences related to replication and integration.

In some embodiments, a construct disclosed herein is delivered to a tissue or a cell of interest via recombinant adeno-associated virus (rAAV). As used herein, “recombinant adeno-associated virus (rAAV)” refers to a genome editing platform that uses recombinant AAV vectors to enable insertion, deletion, and/or substitution of DNA sequences into the genomes of cells. The rAAV can be delivered to a tissue or a cell of interest. It should be appreciated that rAAVs can be produced by using recombinant methods known in the art.

In some embodiments, a helper-free AAV system is compatible with aspects of the disclosure. In some embodiments, the helper-free AAV system comprises three types of plasmids, including an AAV-ITR-containing plasmid, an AAV Rep-Cap plasmid and an AAV-helper plasmid. In some embodiments, the AAV-ITR-containing plasmid contains two AAV ITRs, a gRNA and dSaCas9-VP64. In some embodiments, the AAV Rep-Cap plasmid contains AAV replication genes and capsid genes. In some embodiments, the AAV-helper plasmid contains genes from adenovirus which mediate AAV replication.

In some embodiments, the rAAV comprises a construct disclosed herein and at least one AAV capsid protein. In some embodiments, the vector or rAAV is delivered to the central nervous system (CNS) of a subject. As used herein, delivering the vector or rAAV to the CNS may include delivering the vector or rAAV to any tissue or cell of interest in the CNS. In some embodiments, delivering the vector or the rAAV to the CNS involves delivering the vector or the rAAV to neuronal tissues or cells. In some embodiments, delivering the vector or the rAAV to the CNS involves delivering the vector or rAAV to the brain. In some embodiments, delivering the vector or rAAV to the CNS involves delivering the vector or rAAV to the spinal cord. In some embodiments, delivering the vector or rAAV to the CNS involves delivering the vector or rAAV to the white and/or gray matter. In some embodiments, the vector or rAAV is delivered to any tissue or cell of interest of a subject that may be suitable for the treatments disclosed herein.

As used in the present disclosure, “delivering” or “administering” a vector or a rAAV can include any method known in the art for delivering or administering a vector, a rAAV or a composition comprising a vector or a rAAV to a subject. Administering can include but is not limited to direct administration of a vector or a rAAV or a composition comprising the vector or the rAAV, or peripheral administration via passive diffusion or convection-enhanced delivery (CED) to bypass the blood brain barrier as known in the art. Vectors of rAAVs described herein can be administered in any composition that would be compatible with aspects of the disclosure.

In some embodiments, the capsid protein of the rAAVs can include any known AAV serotype, including, for example, AAVI, AAV2, AAV3, AAV4, AAV5, AAV6, AAV6.2, AAV7, AAV8, AAVrh.8, AAV9, AAV10, AAVrh.10, AAV11, AAVrh39, and AAVrh.43. In some embodiments, the AAV serotype is AAV2. In some embodiments, the AAV serotype is AAV9. Clades of AAV viruses are described in, and incorporated by reference, from Gao et al. (2004) J. Virol. 78 (12): 6381-6388. In some embodiments, any AAV serotype that is suitable for delivery to the CNS may be selected.

AAV vectors of the present disclosure may comprise or be derived from any natural or recombinant AAV serotype. In some embodiments, the AAV vector may utilize or be based on an AAV serotype described in WO 2017/201258A1, the contents of which are incorporated herein by reference in its entirety, such as, but not limited to, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu. 19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2. AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV. AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3. AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-MI, AAV CLv-MII, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVFI/HSC1, AAVFII/HSCII, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8. AAVF9/HSC9, AAV-PHP.B (PHP.B), AAV-PHP.A (PHP.A), G2B-26, G2B-13, TH1.1-32 and/or TH1.1-35, and variants thereof. AAV vectors are described further in U.S. Pat. No. 9,585,971, US 2017/0166926, and WO2020/160337, which are incorporated by reference herein in their entireties.

In some embodiments, the AAV may be packaged into an AAV particle and administered to a subject and/or delivered to a selected target cell. In some embodiments, the AAV particle comprises an AAV capsid protein. In some embodiments, the AAV particle comprises at least one capsid protein that is selected from the AAV serotypes disclosed herein including AAV1, AAV2, AAV3, AAV4, AAV5, AAV6, AAV6.2, AAV7, AAV8, AAV9, PHB.eB, AAV.rh8, AAV.rh10, AAV.rh39, AAV.43, AAV2/2-66, AAV2/2-84, and AAV2/2-125, or a variant of any of the foregoing.

As one of ordinary skill in the art would appreciate, any method known in the art for designing AAV vectors for clinical use, and for delivery of AAV vectors, may be compatible with aspects of the disclosure. For example, non-limiting examples of disclosure related to AAV vectors and delivery are provided in and incorporated by reference from U.S. Pat. No. 7,906,111, entitled “Adeno-associated virus (AAV) clades, sequences, vectors containing same, and uses therefor” and U.S. Pat. No. 9,834,788, entitled “AAV-vectors for use in gene therapy of choroideremia,” each of which is incorporated by reference herein in its entirety.

In some embodiments, the vector is a non-viral vector. For example, non-viral vectors may include, but are not limited to naked DNA, microbubble, plasmids, and episomes.

In some embodiments, constructs described herein can be delivered by any other means known to one of ordinary skill in the art, such as by using liposome nanoparticles, virus-like particles (VLPs), antibody-based delivery systems and/or polyamidoamine dendrimers.

In some embodiments, the viral or non-viral vectors, or other means of delivery, can be any platform or method that can be delivered to the brain, and/or which can cross the blood brain barrier (BBB), and/or which may be suitable for treatments disclosed herein. It has been shown that the BBB is one of the major obstacles for systemically administered vectors or viral vectors to target brain tissues in gene therapies. In some embodiments, a viral or non-viral vector may be modified to enhance the delivery of the viral or non-viral vector across the BBB. For example, capsids of adenoviral vectors may be genetically or chemically modified. Maleimide-activated full-length human transferrin (hTf) may be covalently attached to cysteine-modified adenovirus serotype 5 vectors to its fiber or hexon protein. Any suitable modification of viral or non-viral vectors may be used for improving the capabilities of the viral or non-viral vectors for crossing the BBB.

In some embodiments, a viral vector and a non-viral vector may be used concomitantly for delivering constructs described herein. In some embodiments, a viral vector may be used prior to a non-viral vector for delivering constructs described herein. In some embodiments, a viral vector may be used subsequent to a non-viral vector for delivering constructs described herein. In some embodiments, one or more non-viral vectors may be used.

In some embodiments, a viral and/or non-viral vector and another means of delivery (e.g., liposome nanoparticles, virus-like particles (VLPs), antibody-based delivery systems and/or polyamidoamine dendrimers) may be used concomitantly for delivering constructs described herein. In some embodiments, a viral and/or non-viral vector may be used prior to another means of delivery (e.g., liposome nanoparticles, virus-like particles (VLPs), antibody-based delivery systems and/or polyamidoamine dendrimers) for delivering constructs described herein. In some embodiments, a viral or non-viral vector may be used subsequent to another means of delivery (e.g., liposome nanoparticles, virus-like particles (VLPs), antibody-based delivery systems and/or polyamidoamine dendrimers) for delivering constructs described herein.

### Diseases and Disorders

The present disclosure provides compositions and methods suitable for treating a neurodevelopmental disorder, such as an autism spectrum disorder (ASD), or Phelan-McDermid syndrome.

As used herein “neurodevelopmental disorder” refers to any disorder that impairs the growth and/or development of the brain and/or central nervous system. In some embodiments, neurodevelopmental disorders impact one or more brain functions, such as emotion, learning ability, self-control, and memory. It should be appreciated that aspects of the disclosure may be applicable for treatment of any neurodevelopmental disorder.

In some embodiments, the neurodevelopmental disorder is an autism spectrum disorder (ASD). Diagnosis of ASDs is mainly based on criteria such as deficits in communication, impaired social interaction, and repetitive or restricted interests and behaviors. ASDs are highly heritable disorders with concordance rates as high as 90% for monozygotic twins. However, ASDs are clinically heterogeneous, covering a wide range of discrete disorders of differential symptomatic severity. ASDs are believed to be etiologically heterogeneous, possibly encompassing polygenic, monogenic and environmental factors.

Alterations in synaptic connectivity and function have been proposed as a key mechanism underlying ASDs. Recent genetic studies have identified a large numbers of candidate genes for ASDs, many of which encode synaptic proteins including Shank3, Neuroligin-3, Neuroligin-4 and Neurexin-1. These findings suggest that synaptic dysfunction may underlie a common mechanism for a subset of ASDs. Various Shank3 mutations have been identified as a monogenic cause of ASD with intellectual disability (ID). In ASD patients, all Shank3 deletions and/or mutations that have been identified lead to loss of function (LoF) in one of the two normal copies of the Shank3 gene (i.e., haploinsufficiency). Recent genetic screens also identified a large number of mutations in the Shank3 gene including microdeletions, nonsense mutations and recurrent breakpoints in ASD patients not diagnosed with Phelan-McDermid syndrome (PMS). These implicate Shank3 gene disruption and/or mutation as a monogenic cause of ASD. The current estimation is that deletions and/or mutations involving Shank3 account for about 2% of all ASD patients with ID. Thus, understanding the function of Shank3 may provide insight into pathological mechanisms of ASD.

As used herein, “intellectual disability” refers to a disability that causes a subject to have deficits in intellectual functioning and/or adaptive functioning. Intellectual functioning can include, for example, reasoning, problem solving, planning, abstract thinking, judgment, academic learning, and/or experiential learning. Intellectual functioning can be measured using any method known in the art, such as by IQ tests. Adaptive functioning can include, for example, skills needed to live in an independent and responsible manner such as communication and social skills. In some instances, intellectual disability can be evident during childhood or adolescence.

In some embodiments, the neurodevelopmental disorder is Phelan-McDermid syndrome (PMS, 22q13.3 deletion syndrome), which is an autism spectrum disorder that shows autistic-like behaviors, hypotonia, severe intellectual disability and impaired development of speech and language. Shank3 is one of the genes that has been reported to be deleted in Phelan-McDermid syndrome. Disruption of Shank3 is thought to be the cause of the core neurodevelopmental and neurobehavioral deficits in Phelan-McDermid syndrome because individuals carrying a ring chromosome 22 with an intact Shank3 gene are phenotypically normal.

Other neurodevelopmental disorders can include but are not limited to attention-deficit/hyperactivity disorder (ADHD), learning disabilities such as dyslexia or dyscalculia, intellectual disability (mental retardation), conduct or motor disorders, cerebral palsy, impairments in vision and hearing, developmental language disorder, neurogenetic disorders such as Fragile X syndrome, Down syndrome, Rett syndrome, hypogonadotropic hypogonadal syndromes, and traumatic brain injury.

Neurodevelopmental disorders can be diagnosed or determined by various behavioral assays. For example, behavioral assays may include, but are not limited to, attentional set-shifting test (AST), Elevated-Plus Maze (EPM), social interaction test, and Shock Probe Defensive Burying Test (SPDB). It should be appreciated that any assay or test that may be suitable for assessing behaviors associated with neurodevelopmental disorders may be compatible with aspects of the disclosure.

The present disclosure provides compositions and methods suitable for treating any diseases or disorders that are associated with disrupted Shank3 gene expression, including conditions, signs, or symptoms that are caused by disrupted Shank3 gene expression such as Shank3 gene haploinsufficiency. The disease and disorder may or may not be diagnosed by a medical professional as a neurodegenerative disease.

### Subjects

A subject to be treated by methods described herein may be a human subject or a non-human subject. Non-human subjects include, for example: non-human primates; farm animals, such as cows, horses, goats, sheep, and pigs; pets, such as dogs and cats; and rodents.

A subject to be treated by methods described herein may be a subject having, suspected of having, or at risk for developing a neurodevelopmental disorder. In some embodiments, a subject has been diagnosed as having a neurodevelopmental disorder, while in other embodiments, a subject has not been diagnosed as having a neurodevelopmental disorder. In some embodiments, the subject is a human subject having, suspected of having, or at risk for developing an ASD. In some embodiments, the subject is a human subject having, suspected of having, or at risk for developing Phelan-McDermid syndrome. In some embodiments, the subject has, is suspected of having, or is at risk of having, reduced expression of the Shank3 gene relative to a control subject.

In some embodiments, the expression of the Shank3 gene is reduced in the subject by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. In some embodiments, the control subject is a subject that does not have, is not suspected of having, or is not at risk of having, a neurodevelopmental disorder, an ASD, and/or Phelan-McDermid syndrome. In some embodiments, the reduced expression of the Shank3 gene in a subject is caused by disruption of at least one copy of the Shank3 gene. In some embodiments, the disruption of the Shank3 gene comprises a deletion in at least one copy of the Shank3 gene. In some embodiments, the disruption of the Shank3 gene comprises one or more mutations within at least one copy of the Shank3 gene.

In some embodiments, the subject is a human subject who exhibits one or more symptoms of an ASD. In some embodiments, the subject is a human subject who exhibits developmental delay. In some embodiments, the subject is a human subject who exhibits intellectual disability (ID). In some embodiments, the subject is a human subject who exhibits sleep disturbance. In some embodiments, the subject is a human subject who exhibits hypotonia. In some embodiments, the subject is a human subject who exhibits lack of speech. In some embodiments, the subject is a human subject who exhibits language delay. In some embodiments, the subject is a human subject who exhibits any symptoms or signs of an ASD.

In some embodiments, a subject is a human subject who is an adult. In some embodiments, the adult is older than 25 years of age. In some embodiments, the adult is not older than 25 years of age. In some embodiments, the adult is not older than 21 years of age. In some embodiments, the adult is not older than 18 years of age. In some embodiments, the adult is 16 years of age. In some embodiments, a subject is elderly (e.g., 65 years old or older). In some embodiments, the adult can be any age of adulthood that may be suitable for the treatment disclosed herein.

In some embodiments, the subject is a human subject who is not an adult. In some embodiments, the human subject is not older than 16 years of age. In some embodiments, the human subject is not older than 10 years of age. In some embodiments, the human subject is 10 years of age or younger. In some embodiments, the human subject is a child, an infant, or a newborn. In some embodiments, the human subject is a child older than 1 year old. In some embodiments, the human subject is a toddler. In some embodiments, the human subject is at the fetal stage of development. In some embodiments, the human subject is at the prenatal stage of development.

### Compositions and Administration

The present disclosure provides compositions, including pharmaceutical compositions, comprising a construct delivered in an AAV vector or an rAAV as disclosed herein and a pharmaceutically acceptable carrier. The compositions of the disclosure may comprise an AAV alone, or in combination with one or more other viruses.

Suitable carriers may be readily selected by one of ordinary skill in the art in view of the indication for which the AAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure. Pharmaceutical compositions comprising AAV vectors are described further in U.S. Pat. No. 9,585,971 and US 2017/0166926, which are incorporated by reference herein in their entireties.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the AAV vector or rAAV delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the AAV vectors disclosed herein. The formation and use of liposomes are generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of ML Vs results in the formation of small unilamellar vesicles (SUVs) with diameters in the range of 200 to 500 Å, containing an aqueous solution in the core.

Alternatively, nanocapsule formulations of the AAV vector may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In some embodiments, the pharmaceutical composition comprising a construct as disclosed herein delivered in an AAV vector or rAAV comprises other pharmaceutical ingredients, such as preservatives, or chemical stabilizers. Suitable exemplary preservatives include chlorobutanol, potassium sorbate, sorbic acid, sulfur dioxide, propyl gallate, parabens, ethyl vanillin, glycerin, phenol, thimerosal, and parachlorophenol. Suitable chemical stabilizers include gelatin and albumin. In many cases, it will be preferable to include isotonic agents, for example, sugars or sodium chloride. Prolonged absorption of the pharmaceutical compositions can be brought about by the use in the compositions of agents delaying absorption, for example, aluminum monostearate and gelatin.

The pharmaceutical forms suitable for delivering the AAV vectors or rAAVs include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. In some embodiments, it must be stable under the conditions of manufacture and storage and must be preserved against the contaminating action of microorganisms, such as bacteria and fungi.

Methods described herein comprise administering an AAV vector or rAAV in sufficient amounts to transfect the cells of a desired tissue (e.g., brain) and to provide sufficient levels of gene transfer and expression without undue adverse effects. Conventional and pharmaceutically acceptable routes of administration include, but are not limited to, direct delivery to the selected organ, oral, inhalation, intraocular, intravenous including facial vein injection and retroorbital injection, intracerebroventricular (ICV), intracisterna magna (ICM) injection, intramuscular, intrathecal, intracranial, subcutaneous, intradermal, intratumoral, and other parental routes of administration. Routes of administration may be combined, if desired. In some embodiments, the vector or rAAV as disclosed herein is administered intracerebroventricularly. In some embodiments, the vector or rAAV as disclosed herein is administered intravenously.

In some embodiments, the present disclosure provides methods of upregulating Shank3 gene expression in a subject in need thereof. In some embodiments, the present disclosure provides methods of treating a subject having a neurodevelopmental disorder. In some embodiments, the present disclosure provides methods of treating a subject having an ASD. In some embodiments, the present disclosure provides methods of treating a subject having Phelan-McDermid syndrome. Methods provided herein, in some embodiments, comprise administering and delivering an effective amount of a construct that can be used to upregulate Shank3 gene expressing using CRISPR-mediated activation. In some embodiments, the target tissue is cortex. In some embodiments, the target tissue is striatum. In some embodiments, the target tissue is thalamus cerebellum. In some embodiments, the target tissue is hippocampus. In some embodiments, the target tissue is brain stem. In some embodiments, the target tissue is any brain structure that expresses Shank3.

In some embodiments, methods for administering and/or delivering an effective amount of a composition comprising a vector or an rAAV that comprises a construct as disclosed herein to a target environment or tissue comprise delivering the composition to neurons or other brain cell types. In some embodiment, the vector is an AAV vector. In some embodiments, methods for delivering a construct to a target environment or tissue of a subject in need thereof comprise providing a composition comprising an AAV vector or AAV comprising a construct as disclosed herein to be delivered to the target environment or tissue of the subject and administering the composition to the subject. Methods of use of AAV vectors are described further in U.S. Pat. No. 9,585,971, US 2017/0166926, and WO2020/160337, which are incorporated by reference herein in their entireties.

In some embodiments, the composition comprising a vector or rAAV that comprises a construct as disclosed herein is delivered to a subject via intravenous administration, intracisternal magna injection, systemic administration, intracerebroventricular administration, in utero administration, intrathecal administration, retro-orbital injection, or facial vein injection. In some embodiments, in utero administration is used for a subject who is at the prenatal stage of development. In some embodiments, the composition is delivered to a subject via a nanoparticle. In some embodiments, the composition is delivered to a subject via a viral vector. In some embodiments, the composition is delivered to a subject via any carriers suitable for delivering nucleic acid materials.

In addition to the methods of delivery described above, the following techniques are also contemplated as alternative methods of delivering the AAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

The dose of rAAV comprising a construct as described herein required to achieve a particular “therapeutic effect,” e.g., the units of dose in absolute vector genomes (vg) or vector genomes per milliliter of pharmaceutical solution (vg/mL) will vary based on several factors including, but not limited to: the route of AAV administration, the level of gene expression required to achieve a therapeutic effect, the specific disease or disorder being treated, and the stability of the gene product. Doses that give maximal percentage of infection without affecting neurodevelopment are also suitable. One of skill in the art can readily determine a AAV dose range to treat a patient having a particular disease or disorder based on the aforementioned factors, as well as other factors.

An effective amount of rAAV is an amount sufficient to infect an animal or human subject or target a desired tissue. The effective amount will depend primarily on factors such as the species, age, gender, weight, health of the subject, and the tissue to be targeted, and may thus vary among subjects and tissues. The term “effective amount” or “amount effective” in the context of a composition or dose for administration to a subject refers to an amount of the composition or dose that produces one or more desired responses in the subject. In some embodiments, an effective amount of a composition disclosed herein may partially or fully rescue the effects of a mutated Shank3 gene and/or partially or fully restore loss of function of the Shank3 protein. An effective amount can involve reducing the level of an undesired response, although in some embodiments, it involves preventing an undesired response altogether. An effective amount can also involve delaying the occurrence of an undesired response. An effective amount can also be an amount that produces a desired therapeutic endpoint or a desired therapeutic result. In other embodiments, the amounts effective can involve enhancing the level of a desired response, such as a therapeutic endpoint or result. The achievement of any of the foregoing can be monitored by routine methods and the methods as disclosed in the present application. Effective amounts will depend, of course, on the particular subject being treated; the severity of a condition; the individual patient parameters including age, physical condition, size and weight; the duration of the treatment; the nature of concurrent therapy (if any); the specific route of administration and like factors. It should be appreciated that an effective amount, as used herein, does not need to be a clinically effective amount.

The efficacy of the effective amounts can be determined by evaluating Shank3 gene or protein expression. The efficacy of the effective amounts can be determined by behavior analysis. The efficacy of the effective amounts can be determined by the levels of the biomarkers associated with Shank3.

For example, in some embodiments, the number of vector genomes administered to the subject is any value between about 6.0×10¹¹ vg and about 9.0×10¹³ vg. In some embodiments, the number of vector genomes administered to the subject is any value between about 6.0×10¹³ vg/mL and about 9.0×10¹³ vg. In some embodiments, the number of vector genomes administered to the subject is any value between about 1×10¹⁰ to about 1×10¹⁴ vg. In certain embodiments, the effective amount of rAAV is 10¹⁰, 10¹¹, 10¹², 10¹³, or 10¹⁴ genome copies per kg. In certain embodiments, the effective amount of rAAV is 10¹⁰, 10¹¹, 10¹², 10¹³, 10¹⁴, or 10¹⁵ genome copies per subject. In some cases, a dosage between about 101 to 1013 genome copies is appropriate. In some embodiments, the number of vector genomes administered to the subject can be any dose that may be suitable for the treatments and methods disclosed herein.

In some embodiments, a dose of vector or rAAV is administered to a subject no more than once per calendar day (e.g., a 24-hour period). In some embodiments, a dose of vector or rAAV is administered to a subject no more than once per 2, 3, 4, 5, 6, or 7 calendar days. In some embodiments, a dose of vector or rAAV is administered to a subject no more than once per calendar week (e.g., 7 calendar days). In some embodiments, a dose of vector or rAAV is administered to a subject no more than bi-weekly (e.g., once in a two calendar week period). In some embodiments, a dose of vector or rAAV is administered to a subject no more than once per calendar month (e.g., once in 30 calendar days). In some embodiments, a dose of vector or rAAV is administered to a subject no more than once per six calendar months. In some embodiments, a dose of vector or rAAV is administered to a subject no more than once per calendar year (e.g., 365 days or 366 days in a leap year). In some embodiments, a dose of vector or rAAV is administered to a subject no more than once per two calendar years (e.g., 730 days or 731 days in a leap year). In some embodiments, a dose of vector or rAAV is administered to a subject no more than once per three calendar years (e.g., 1095 days or 1096 days in a leap year).

Formulation of pharmaceutically-acceptable excipients and carrier solutions disclosed herein is well-known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, administration of a construct or a composition comprising a construct can upregulate Shank3 gene expression to wild-type levels. In some embodiments, administration of a construct or a composition comprising a construct can upregulate Shank3 gene expression by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. In some embodiments, a control subject is a subject that does not receive the construct or composition.

In some embodiments, administration of a construct or a composition comprising a construct described herein to upregulate Shank3 gene expression can lead to improving sleep efficiency. In some embodiments, a subject has improved sleep efficiency after being administered an effective amount of a construct or a composition comprising a construct. In some embodiments, the sleep efficiency in the subject after being administered an effective amount of the construct or composition comprising a construct is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. In some embodiments, a control subject is a subject that does not receive the construct or composition comprising a construct. Improved sleep efficiency includes less sleep disturbance, which includes but is not limited to having trouble falling and staying asleep. Measurement of sleep efficiency can be conducted using any methods known in the art.

In some embodiments, administration of a construct or a composition comprising a construct described herein to upregulate Shank3 gene expression can lead to improving social impairment. In some embodiments, the social impairment of the subject is improved after being administered an effective amount of a construct or a composition comprising a construct. In some embodiments, the social impairment in the subject after being administered an effective amount of the construct or composition comprising a construct is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. In some embodiments, a control subject is a subject that does not receive the construct or composition comprising a construct. Measurement of social impairment can be conducted using any methods known in the art.

As used herein, “social impairment” refers to behavioral abnormalities or defects that prohibit a subject from displaying voluntary social interaction.

In some embodiments, administration of a construct or a composition comprising a construct described herein to upregulate Shank3 gene expression can lead to improving locomotion and/or motor coordination deficits. In some embodiments, the locomotion and/or motor coordination deficits of the subject are improved after being administered an effective amount of the construct or composition comprising a construct. In some embodiments, the locomotion and/or motor coordination deficits in the subject after being administered an effective amount of the construct or composition comprising a construct is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. In some embodiments, a control subject is a subject that does not receive the construct or composition comprising a construct. Measurement of locomotion and/or motor coordination deficits can be conducted using any methods known in the art.

As used herein, “locomotion and/or motor coordination deficits” can include, for example, lack of coordination, loss of balance, and/or a shuffling gait.

In some embodiments, administration of a construct or a composition comprising a construct described herein to upregulate Shank3 gene expression can lead to improvement in cortical-striatal synaptic dysfunction. In some embodiments, the cortical-striatal synaptic dysfunction of the subject is improved after being administered an effective amount of the construct or composition comprising a construct. In some embodiments, the cortical-striatal synaptic dysfunction in the subject after being administered an effective amount of the construct or composition comprising a construct is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. In some embodiments, a control subject is a subject that does not receive the construct or composition comprising a construct. Measurement of cortical-striatal synaptic dysfunction can be conducted using any methods known in the art.

As used herein, “cortical-striatal synaptic dysfunction” refers to defective corticostriatal circuits in the brain that can cause repetitive and compulsive behaviors, such as in neuropsychiatric disorders and neurodevelopmental diseases such as autism, obsessive-compulsive disorders, and Tourette syndrome.

Some aspects of the technology described herein may be understood further based on the non-limiting illustrative embodiments described in the below Examples section. Any limitations of the embodiments described in the below Examples section are limitations only of the embodiments described in the below Examples section and are not limitations of any other embodiments described herein.

### Examples

In order that the invention described herein may be more fully understood, the following examples are set forth. The examples described in this application are offered to illustrate the systems and methods provided herein and are not to be construed in any way as limiting their scope.

### Example 1: Design and Optimization of a CRISPR/dSaCas9 Transcription Activator System for Increasing Shank3 Gene Expression

Transcriptional activation methods using a CRISPR-mediated activating system were developed to achieve gene upregulation in models of haploinsufficiency. The Shank3 gene displays an extensive array of mRNA and protein isoforms resulting from the combination of multiple intragenic promoters and extensive alternative splicing of coding exons in the mouse brain. The longest isoform of Shank3 plays critical roles in the etiology of ASD.

A system was developed that includes a nuclease-dead Cas9 protein from S. aureus (dSaCas9) fused to a weak transcriptional activator VP64, targeted to the Shank3 promoter region with a guide RNA. VP64 is a transcriptional activator that carries four tandem copies of VP16 (a herpes simplex virus type 1 transcription factor). It is also known to have a moderate activation potential compared to other known activators for a wide variety of genes, which could be advantageous in obtaining physiologically relevant Shank3 dosage levels in vivo. In order to increase expression of the wildtype Shank3 gene (FIG. 1A), CRISPR activation (CRISPRa) conditions were optimized in vitro. This model served as the basis for testing potential Shank3 therapeutics (FIGS. 1A-1B).

Seven small guide RNAs (gRNAs) were designed to target the Shank3 promoter which drives the expression of the longest isoform of Shank3. These gRNAs were referred to as Shank3-gRNA1-7. The relative position of the gRNAs to the transcriptional start site (TSS) of Shank3 gene is shown in FIG. 1C. Neuro2a cells, a mouse neuroblastoma cell line, was chosen to test the Shank3 CRISPRa system in vitro because weak Shank3 expression could be detected in these cells. Transfection conditions were optimized to reach ˜50˜60% transfection rate using a pEF1-EGFP construct (FIG. 1D). The Neuro2a cells were then transfected with Shank3 CRISPRa guide RNAs. After 48 hours, Shank3 mRNA levels were measured using quantitative PCR (qPCR). This approach successfully identified gRNAs for the Shank3 promoter that were able to upregulate endogenous Shank3 mRNA from 2- to 13-fold (FIG. 1E; gRNA1: 2.91±0.79, gRNA2: 3.84±0.20, gRNA3: 12.65±0.57, gRNA4: 4.87±0.76, gRNA5: 3.89±0.38 gRNA6: 5.84±0.35, gRNA7: 2.19±0.12). Further analysis was primarily focused on the Shank3-gRNA3, which resulted in the highest amount of Shank3 upregulation.

### Example 2: Delivery of Shank3 CRISPRa rAAV to Mouse Brains Upregulates Shank3 in Shank3-InG3680^{+/-} Mice

To investigate the translational potential of the Shank3 CRISPRa system described herein to upregulate Shank3 in mice, recombinant adeno-associated virus (rAAV) was used to deliver CRISPRa into the brains of an ASD mouse model, Shank3-InG3680^{+/-} mice. The Shank3-InG3680^{+/-} mouse line harbors the ASD patient-linked single guanine nucleotide (G) insertion at DNA position 3680, which leads to a frameshift and downstream stop codon (InsG3680 mutation). It was previously found that the Shank3-InG3680^{+/-} mouse line manifests defective synaptic transmission in the striatum before weaning age, as well as impaired juvenile social play behavior, coinciding with the early onset of ASD symptoms in human patients. This model served as the basis for further testing the CRISPRa system for upregulating Shank3 using recombinant adeno-associated virus (rAAV).

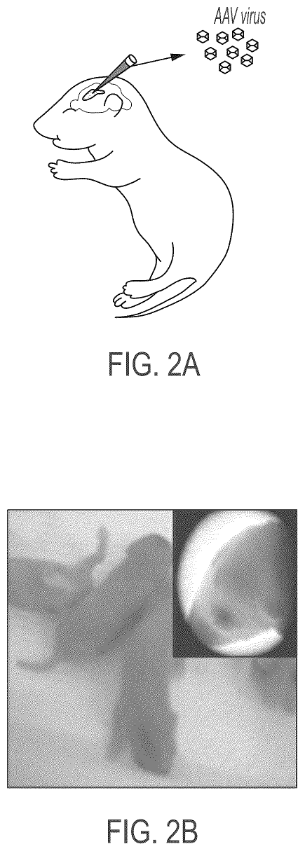

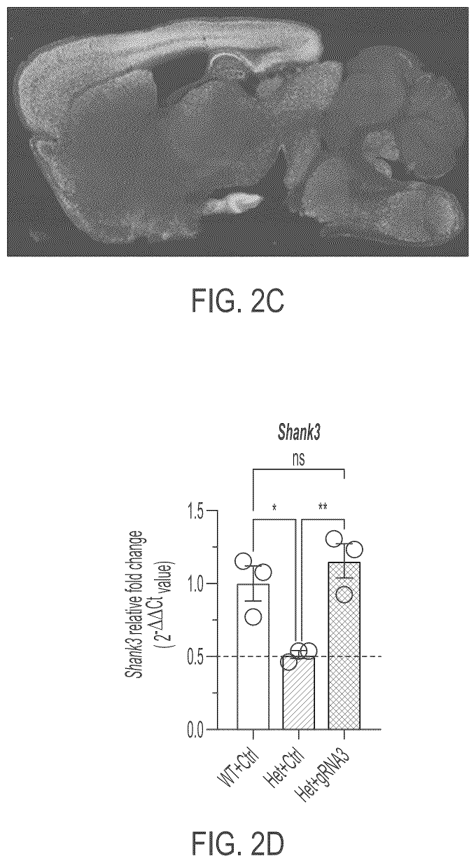

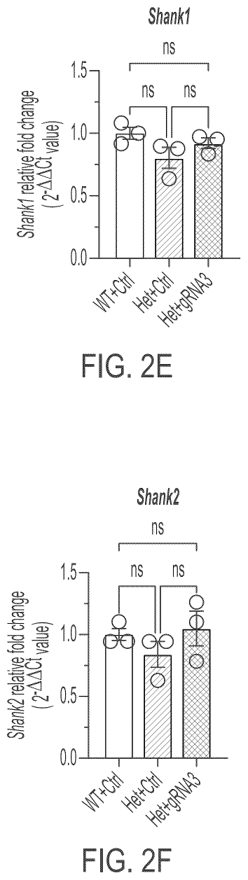

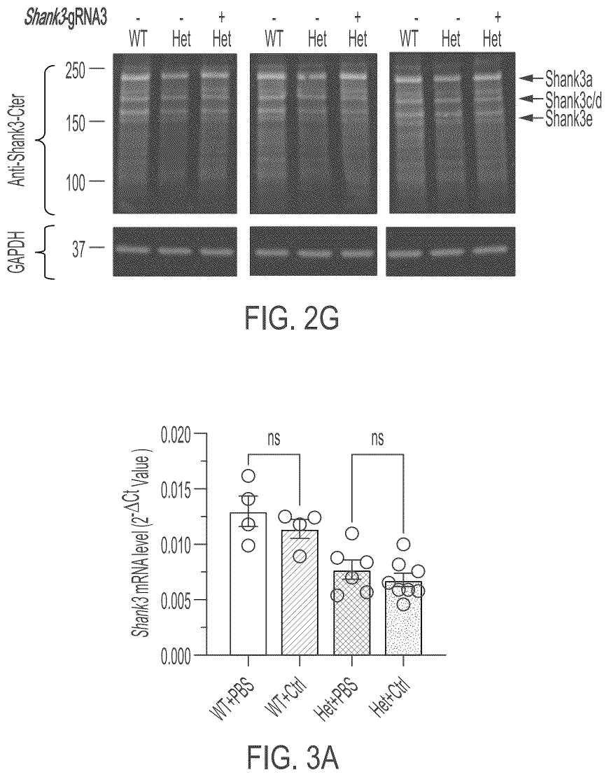

Intracerebroventricular injection (ICV injection; FIG. 2A) was used to deliver the rAAVs into developing brains. ICV injection conditions were optimized based on expression of EGFP fluorescence in mice injected with AAV-hSyn-nlsEGFP, which is an AAV containing nuclear-localized EGFP driven by human Synapsin/promoter (hSyn). Strong EGFP fluorescence signals were observed in most brain areas one-month post-ICV injection (FIGS. 2B-2C). Shank3-CRIPSPa constructs were then packaged into rAAV2/9 serotype. CRISPRa using Shank3-gRNA3 was tested (pAAV-U6-Shank3-gRNA3-CMV-dSaCas9-VP64). Virus carrying pAAV-U6-Shank3-gRNA3-CMV-dSaCas9-VP64 was administered by stereotactic injection into the left and right ventricles of Shank3-InG3680^{+/-}-mice at postnatal day 1 (P1) using the ICV injection method.

To test whether Shank3 expression levels were increased by delivering CRISPRa-rAAV to the brains of Shank3-InG3680^{+/-}-mice, mRNA expression levels for Shank3 from one-month-old AAV injected mice were measured. Shank3 upregulation to wild type (WT) levels was observed in Shank3-InG3680^{+/-} mice injected with rAAVs containing Shank3-gRNA3 CRISPRa, but not in mice injected with control virus (AAV-U6-CMV-dSaCas9-VP64) (FIG. 2D). To test the specificity of Shank3-gRNA3 CRISPRa mediated upregulation of Shank3. mRNA expression levels for other Shank family members, Shank1 and Shank2, were measured from one-month-old AAV injected mice and were found to be unaffected by Shank3-gRNA3 CRISPRa expression (FIGS. 2E-2F). It was next investigated whether Shank3-gRNA3 CRISPRa-mediated upregulation of Shank3 mRNA could lead to the upregulation of Shank3 protein. Western blot analysis showed that the Shank3 protein was increased to the WT level in Shank3-InG3680^{+/-} mice injected with Shank3-gRNA3 CRISPRa, but not in mice injected with control virus (FIG. 2G). Only the longest isoform of Shank3 protein was significantly upregulated. These results showed that Shank3-gRNA3 CRISPRa could significantly increase both mRNA and protein expression from the wild type allele of Shank3-InG3680^{+/-}-mice.

### Example 3: Long-Term Expression of Shank3 CRISPRa rAAV in Mouse Brains

Most genes are dosage-sensitive and either deletions or duplications are detrimental. For example, it has been reported that overexpression of Shank3 could increase neuronal activity and could lead to behavioral seizures.

In the Shank3 CRISPRa system described above, both the gRNA and dSaCas9-VP64 driven by ubiquitous promoters, U6 and CMV. To examine whether long-term expression of the Shank3 CRISPRa system could upregulate Shank3 beyond the WT level, gRNA-containing Shank3 CRISPRa rAAVs that were found to upregulate Shank3 mRNA in Neuro-2a cells at various levels were delivered to mouse brain by ICV injection (including gRNA2, gRNA3, gRNA6 and gRNA7).

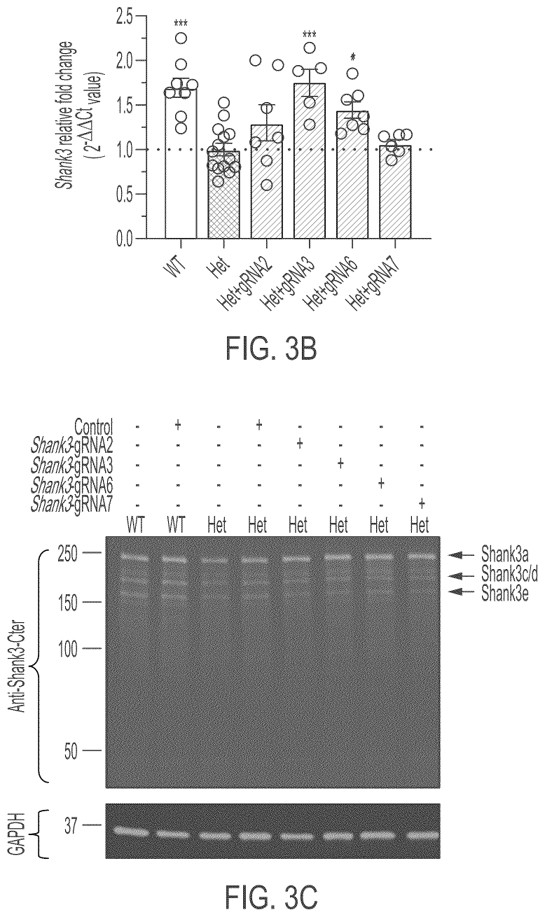

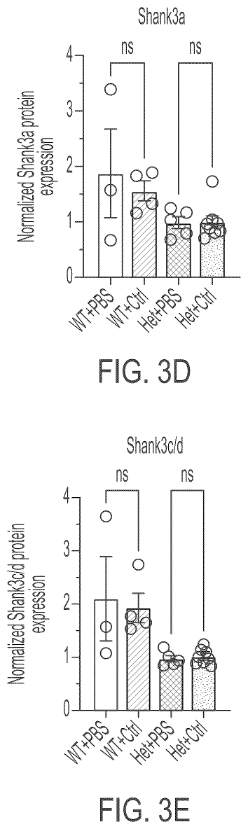

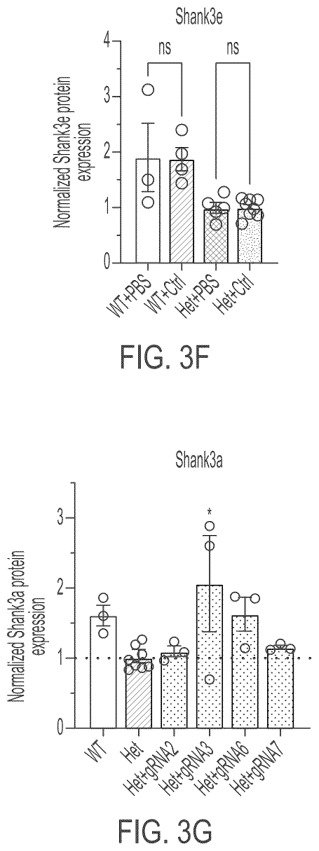

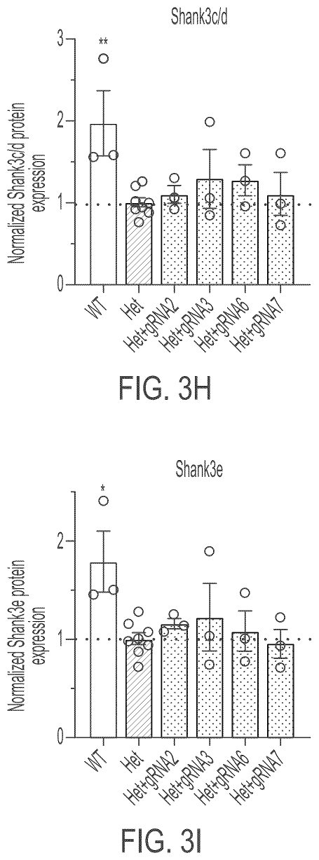

Moreover, potential non-specific activation by the transcriptional activator, VP64 was investigated. To rule out the possibility that Shank3 gene upregulation was caused by non-specific effects of VP64, control rAAVs were injected into both WT and Shank3-InG3680^{+/-} mouse brains by ICV. Compared to mice with ICV-injections of PBS, the control rAAVs could not increase either the Shank3 mRNA or protein levels in both WT and Shank3-InG3680″ mice 3˜4 months after the ICV injection (FIGS. 3A, and C-F).

Interestingly, in mice injected with Shank3-CRISPRa rAAVs, even the most effective gRNA3-containing Shank3 CRISPRa rAAVs could only upregulate Shank3 mRNA and protein levels to WT level (FIGS. 3B, C and G-I). Since the Shank3 CRISPRa system uses an exogenous transcriptional activator to upregulate Shank3 transcription from the normal copy of the Shank3 gene in Shank3-InG3680^{+/-} heterozygotes and significant over expression was not observed, these results suggest that the exogenous transcription activation system is also tightly controlled by endogenous regulatory mechanisms in cells.

### Example 4: Shank3 CRISPRa rAAV can Upregulate Shank3 in Shank3-InG3680^{+/-} Mice in Different Brain Regions

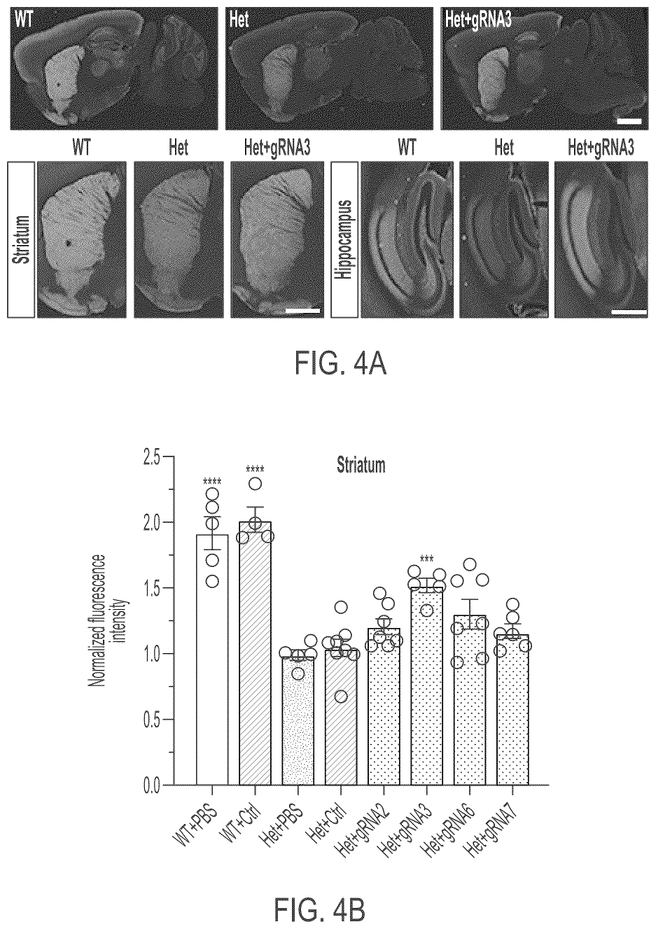

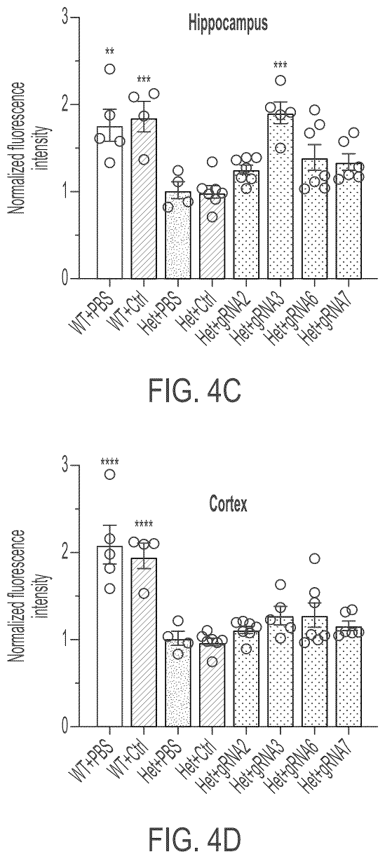

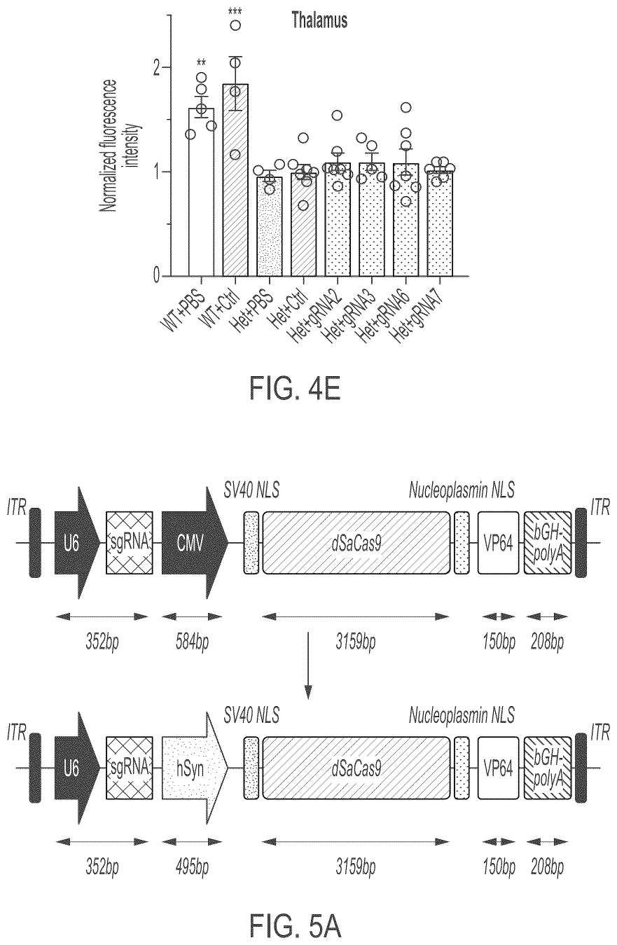

Shank3 is highly expressed in the striatum and modestly expressed in cortex, hippocampus, thalamus and cerebellum. Shank3 expression is reduced in Shank3-InG3680^{+/-} mutant mice (FIG. 4A). In the Shank3 CRISPRa system, both the gRNA and dSaCas9-VP64 are driven by ubiquitous promoters, U6 and CMV, and therefore the Shank3 CRISPRa system could be expressed in different cell types and brain regions with the ICV injection method. To examine the upregulation of Shank3 in different brain regions, immunohistochemistry was conducted after the ICV injection of Shank3 CRISPRa rAAVs. Compared to the Shank3-InG3680^{+/-} mice, the gRNA3-containing Shank3 CRISPRa could significantly increase the Shank3 expression in striatum and hippocampus of Shank3-InG3680^{+/-}-mice 3-4 months after the ICV injection, but not in cortex and thalamus (FIGS. 4A-4E). These results suggest that there is not ectopic expression of Shank3 in the brain after Shank3 CRISPRa ICV-injection.

### Example 5: CRISPRa Shank3 Gene Induction in Mouse Brains Under Neuronal Specific Human Synapsin-1 Promoter

Although CMV-driven Shank3 CRISPRa did not result in ectopic expression of Shank3 in mice after the Shank3 CRISPRa ICV-injection, there is still a potential risk of CMV-driven Shank3 CRISPRa in future clinic applications in human patients. To address this, the CMV promoter was swapped with human Synapsin/promoter. Human Synapsin1 is a widely used neuronal specific promoter that restricted expression of the Shank3 CRISPRa system to neuronal cells of the brain (FIG. 5A).

To test this neuron specific Shank3 CRISPRa system, ICV-injection of rAAVs was conducted in P1 mouse brains. Neuronal-specific Shank3 CRISPRa rAAVs were able to significantly increase Shank3 mRNA and protein expression of Shank3-InG3680^{+/-} mice 3-4 months after the ICV injection (FIGS. 5B-5C).

Immunohistochemistry results showed that the gRNA3-containing Shank3 CRISPRa could significantly increase Shank3 expression in striatum and hippocampus, but not in cortex and thalamus, which is similar to the non-neuronal specific Shank3 CRISPRa (FIGS. 5D-5H).

### Example 6: Materials, Methods, and Study Design for Examples 1-5

### RNA Isolation Quantitative Reverse-Transcription PCR

RNA was isolated from cells or tissues using Trizol reagent (Invitrogen, USA) following the manufacture's protocol. For mice, animals were euthanized, and tissues were harvested directly into the Trizol reagent and homogenized. For qRT-PCR, 1000 ng of RNA was reverse transcribed using iScript™ Reverse Transcription Supermix for RT-qPCR kit (BioRad, USA). qPCR was performed using SsoAdvanced Universal SYBR Green Supermix (Bio-Rad) and the Bio-Rad CFX96 thermocycler. Primers were verified with standard curves to ensure reliability. Optimal primer pairs were then used to evaluate levels of cDNA samples. The Primers were:

Shank3-Exon1-RT-F1:
(SEQ ID NO: 35)
5′-CCGGACCTGCAACAAACGA-3′;
Shank3-Exon1-RT-R1:
(SEQ ID NO: 36)
5′-GCGCGTCTTGAAGGCTATGAT-3′;
Shank1-RT-F1:
(SEQ ID NO: 37)
5′-CCGCTACAAGACCCGAGTCTA-3′;
Shank1-RT-R1:
(SEQ ID NO: 38)
5′-CCTGAATCTGAGTCGTGGTAGTT-3;
Shank2-RT-F1:
(SEQ ID NO: 39)
5′-AGAGGCCCCAGCTTATTCCAA-3′;
Shank2-RT-R1:
(SEQ ID NO: 40)
5′-CAGGGGTATAGCTTCCAAGGC-3′;
Gapdh-RT-F1:
(SEQ ID NO: 41)
5′-CATGGCCTTCCGTGTTCCT-3′;
and

Gapdh-RT-R1: 5′-TGATGTCATCATACTTGGCAGGTT-3′ (SEQ ID NO: 42). Genes of interest were normalized to Gapdh and presented as fold changes over baseline using the delta-delta CT method.

### Western Blot

Animals were euthanized, and tissues were harvested directly into RIPA buffer (ThermoFisher) and homogenized. Protein concentration was determined by the BCA assay using BSA as a standard (Pierce). For quantification, samples containing equal amounts of protein (12 μg) were resolved by SDS-PAGE using 4-20% Mini-PROTEAN® TGX™ Precast Protein Gels (BioRad) and subsequently transferred to nitrocellulose membranes. Following transfer, the membrane was blocked with 5% nonfat milk in TBST for 1 hr at room temperature and probed with Shank3 (1:1000, #64555, Cell Signaling Technology, USA) and GAPDH (1:1000, #33233, SantaCruz, USA) antibodies overnight. The membranes were then washed in PBST and incubated with IR-dye-labeled secondary antibodies (1:10,000; LI-COR Biosciences) for 1 h at room temperature, washed again in PBST, and visualized with the Odyssey infrared imaging system (LI-COR Biosciences). Blots were analyzed using Image Studio (LI-COR Biosciences) and normalized to loading control (GAPDH).

### AAV Construct and Virus Production

pCMV-SadCas9-VP64 (Addgene, #115790) was digested with XhoI (NEB) and ligated with U6-gRNA gBLOCK (synthesized by IDT) using HIFI DNA assembly kit

(NEB). The constructs were confirmed with Sanger sequencing.

The AAV production method used was as described in detail previously²⁸. Briefly, the AAV plasmid containing gRNAs, pAdDeltaF6 (Addgene, #112867) and pAAV2/9 (Addgene, #112865) were co-transfected into HEK293T cells using polyethylenimine (Cat. 23966-1; Polysciences). The cells were cultured in Dulbecco's modified essential medium (DMEM; Invitrogen, USA), containing 10% fetal bovine serum (Gibco, USA) and 1% penicillin-streptomycin (Gibco, USA) at 37° C. with 5% CO₂. The cells and media were harvested 72 h post transfection. Cells were harvested by 4,000 g centrifugation at 4° C. for 10 min. The virus in media was precipitated by 8% PEG 8000 (Sigma). Both cell pellet and virus precipitated from media were re-suspended in digestion buffer containing 500 mM NaCl, 40 mM Tris base and 10 mM MgCl₂. Benzonas nuclease (100U, Sigma) was added in digestion buffer and incubated in 37° C. water bath for an hour. Samples were centrifuged at 2,000 g for 15 min and the supernatant was added on a discontinuous gradient of 15%, 25%, 40% and 60% iodixanol in a 36.2 ml ultracentrifuge tube (Optiseal Seal, 362183, Beckman). Samples were ultracentrifugated at 350,000 g, at 18° C. for 2 h 30 min. 5 ml fractions in 40% layer and 40%/60% interface were collected. The fractions were desalted using a 100 kDa cut-off ultrafiltration tube (15 ml; Millipore). The buffer was exchanged 4 times with 1x PBS with 0.001% Pluronic F-68. The AAV titers were determined by real-time quantitative PCR (qPCR) using the primers of SaCas9:

Forward primer:
(SEQ ID NO: 43)
5′-ATCACCCCCCACCAGATCAAGC-3′;
Reverse primer:
(SEQ ID NO: 44)
5′-GTCCTTGTCGTACAGGCCGTTCA-3′.

### Animal Use

All experiments were conducted under the guidelines of the Division of Comparative Medicine, with the protocol approved by the Committee for Animal Care of the Massachusetts Institute of Technology. The Shank3-InG3680″-mouse line was generated as described previously and kept in C57BL6/129 mixed genetic background. WT and Shank3-InG3680^{+/-}-mice used in the experiments were obtained from a WT and Shank3-InG3680″-/Shank3-InG3680^{+/+}/mating strategy. All mice were bred in house on a 12 h light/dark cycle (lights on at 7:00, lights off at 19:00) with food and water available ad libitum. Animal experiments and data analysis were performed with experimenters blinded to genotypes.

### P1 Mouse Intracerebroventricular Injections

ICV injection procedures were performed using established protocols^{29.30}. In brief, P1 pups were induced with hypothermic anesthesia by placement on ice. Anesthesia was confirmed by squeezing of paw and cessation of movement before injections. ICV injections were performed using 10 μl glass pipets. Ventricular injection sites were identified by ⅔ distance from lambda suture to eye and 3 mm ventral from skin (marked on needle shaft). Viral solutions were diluted in PBS at 1×10¹³ gc/ml with fast green dye (1 mg/ml, Sigma, USA) and injected as a 1 μl/hemisphere. Both hemispheres were injected. Equivalent 2×1010 gc/pup. Injected pups were placed on a warming pad and regained movement before being returned to the home cage.

### Immunohistochemistry

Mice were anesthetized with isoflurane and transcardially perfused with PBS followed by 4% paraformaldehyde (PFA, Sigma) in PBS. Brains were dissected out and post-fixed in the 4% PFA at 4° C. overnight. After that, brains were sliced in 50 μm-thick sagittal sections by using a Vibratome VT1000S (Leica Biosystems, Germany). Sections were serially collected in plates containing PBS and were stored at 4° C. for subsequent immunohistochemical staining. Brain slices were rinsed once with PBS and permeated with 0.5% Triton X-100 (Sigma, USA) in PBS for 30 min at room temperature. Then, the slices were rinsed once with PBS and incubated with blocking buffer (containing 15% normal goat serum, 5% BSA and 0.2% Triton X-100) for 1 h at room temperature. After that, slices were incubated with Shank3 antibody (1:400, #64555, Cell Signaling Technology, USA) at 4° C. for 24 h. Then, the slices were washed with PBS with 0.02% Triton X-100 four times at room temperature, each time for 15 min. Slices were incubated room temperature for 2 h with the secondary antibody (#A-21245, Invitrogen). Slices were washed three times in PBS with 0.02% Triton X-100, each time for 15 min. Brain slices were mounted on slides with Fluormount-G mounting medium (Southern Biotech, USA).

### Statistics

All data were transferred to Prism 9.0 (GraphPad Software, Inc., USA) for statistical analysis and graphing. Data are presented as the mean±the s.e.m., and the n value given for each experiment refers to the number of mice analyzed. All error bars indicate the s.e.m. The significance levels for all tests were set at *p<0.05, **p<0.01, ***p<0.001, and ****p<0.0001.

TABLE 1
Representative sequences for Shank3-CRISPRa system

| SEQ ID NO: | Description | Sequence |
|---|---|---|
| 1 | gRNA1 | GCGGTCGGCGGGGCTCTTGCG |
| 2 | gRNA2 | GGAGGGGGCCGCGCCGGAGGC |
| 3 | gRNA3 | CCGGGAAACTGGGGTCCCCCG |
| 4 | gRNA4 | GGGGCCCGCGCCGAAGAGGGG |
| 5 | gRNA5 | GGGGAGTGGCTGACACAGCGG |
| 6 | gRNA6 | GGGTGACGGAGGGTTCTAGAG |
| 7 | gRNA7 | TCTCTCCCTCTCAGTCGGCCT |
| 8 | AAV-U6- | cagcactgcataattctcttactgtcatgccatccgtaagatgcttttctgtgactggtgagtactcaacaagtcattctg |
| | Control-CMV- | agaatagtgtatgcggcgaccgagttgctcttgcccggcgtcaatacgggataataccgcgccacatagcagaacttt |
| | dSaCas9-VP64 | aaaagtgctcatcattggaaaacgttcttcggggcgaaaactctcaaggatcttaccgctgttgagatccagttcgatgt |
| | 5′ part of | aacccactcgtgcacccaactgatcttcagcatcttttactttcaccagcgtttctgggtgagcaaaaacaggaaggca |
| | Ampicillin | aaatgccgcaaaaaagggaataagggcgacacggaaatgttgaatactcatactcttcctttttcaatattattgaagcattt |
| | resistant gene | atcaggcttattctctcatgagcggatacatatttgaatgtatttagaaaaataaacaaataggggttccgcgcacatttccccga |
| | (AmpR) (bold)- | aaagtgccacctgacgtctaagaaaccattattatcatgacattaacctataaaaataggcgtatcacgaggccctttcgtctcgc |
| | AmpR promoter | gcgtttcggtgatgacggtgaaaacctctgacacatgcagctcccggagacggtcacagcttgtctgtaagcggatgccggg |
| | (underlined)-f1 | agcagacaagcccgtcagggcgcgtcagcgggtgttggcgggtgtcggggctggcttaactatgcggcatcagagcagatt |
| | bacteriophage | gtactgagagtgcaccataaaattgtaaacgttaatattttgttaaaattcgcgttaaatttttgttaaatcagctcatttttt |
| | origin of | aaccaatagaccgaaatcggcaaaatcccttataaatcaaaagaatagcccgagatagagttgagtgttgttccagtt |
| | replication | tggaacaagagtccactattaaagaacgtggactccaacgtcaaagggcgaaaaaccgtctatcagggcgatggcc |
| | (bold)-5′ITR | cactacgtgaaccatcacccaaatcaagttttttggggtcgaggtgccgtaaagcactaaatcggaaccctaaagggagc |
| | (underlined)-U6 | ccccgatttagagcttgacggggaaagccggcgaacgtggcgagaaaggaagggaagaaagcgaaaggagcgggcgct |
| | promoter (bold)- | aaggcgctggcaagtgtagcggtcacgctgcgcgtaaccaccacacccgccgcgcttaatgcgccgctacagggcgcgta |
| | gRNA scaffold | ctatggttgctttgacgtatgcggtgtgaaataccgcacagatgcgtaaggagaaaataccgcatcaggcgcccctgcaggca |
| | (underlined)- | gctgcgcgctcgctcgctcactgaggccgccccgccaaagcccgggcgtcgggcgacctttgctcgccccgcctcactga |
| | CMV promoter | gcgagcgagcgcgcagagagggagtggccaactccatcactaggggttcctgcggccgcctcgagGAGGGCCT |
| | (bold)-SV40 | ATTTCCCATGATTCCTTCATATTTGCATATACGATACAAGGCTGTTAGA |
| | nuclear | GAGATAATTGGAATTAATTTGACTGTAAACACAAAGATATTAGTACAAA |
| | localization | ATACGTGACGTAGAAAGTAATAATTTCTTGGGTAGTTTGCAGTTTTAAA |
| | signal | ATTATGTTTTAAAATGGACTATCATATGCTTACCGTAACTTGAAAGTAT |
| | (underlined)- | TTCGATTTCTTGGCTTTATATATCTTGTGGAAAGGACGAAACACCGGAG |
| | dSaCas9 (bold)- | ACCACGGCAGGTCTCAGTTATAGTACTCTGGAAACAGAATCTACTATAACA |
| | Nucleoplasmin | AGGCAAAATGCCGTGTTTATCTCGTCAACTTGTTGGCGAGATTTTTTTGCGT |
| | nuclear | TGACATTGATTATTGACTAGTTATTAATAGTAATCAATTACGGGGTCAT |
| | localization | TAGTTCATAGCCCATATATGGAGTTCCGCGTTACATAACTTACGGTAAA |
| | signal | TGGCCCGCCTGGCTGACCGCCCAACGACCCCCGCCCATTGACGTCAAT |
| | (underlined)- | AATGACGTATGTTCCCATAGTAACGCCAATAGGGACTTTCCATTGACGT |
| | VP64 (bold)- | CAATGGGTGGAGTATTTACGGTAAACTGCCCACTTGGCAGTACATCAA |
| | Bovine growth | GTGTATCATATGCCAAGTACGCCCCCTATTGACGTCAATGACGGTAAAT |
| | hormone | GGCCCGCCTGGCATTATGCCCAGTACATGACCTTATGGGACTTTCCTAC |
| | polyadenylation | TTGGCAGTACATCTACGTATTAGTCATCGCTATTACCATGGTGATGCGG |
| | signal | TTTTGGCAGTACATCAATGGGCGTGGATAGCGGTTTGACTCACGGGGA |
| | (underlined)- | TTTCCAAGTCTCCACCCCATTGACGTCAATGGGAGTTTGTTTTGGCACC |
| | 3′TTR (bold)- | AAAATCAACGGGACTTTCCAAAATGTCGTAACAACTCCGCCCCATTGAC |
| | high-copy- | GCAAATGGGCGGTAGGCGTGTACGGTGGGAGGTCTATATAAGCAGAGC |
| | number | TCTCTGGCTAACTACCGGTGCCACCATGGCCCCAAAGAAGAAGCGGAAGGT |
| | ColE1/pMB1/ | CGGTATCCACGGAGTCCCAGCAGCCAAGCGGAACTACATCCTGGGCCTG |
| | pBR322/pUC | GCCATCGGCATCACCAGCGTGGGCTACGGCATCATCGACTACGAGACA |
| | origin of | CGGGACGTGATCGATGCCGGCGTGCGGCTGTTCAAAGAGGCCAACGTG |
| | replication | GAAAACAACGAGGGCAGGCGGAGCAAGAGAGGCGCCAGAAGGCTGAA |
| | (underlined)-3′ | GCGGCGGAGGCGGCATAGAATCCAGAGAGTGAAGAAGCTGCTGTTCGA |
| | part of | CTACAACCTGCTGACCGACCACAGCGAGCTGAGCGGCATCAACCCCTA |
| | Ampicillin | CGAGGCCAGAGTGAAGGGCCTGAGCCAGAAGCTGAGCGAGGAAGAGT |
| | resistant gene | TCTCTGCCGCCCTGCTGCACCTGGCCAAGAGAAGAGGCGTGCACAACG |
| | (AmpR) (bold) | TGAACGAGGTGGAAGAGGACACCGGCAACGAGCTGTCCACCAAAGAGC |
| | | AGATCAGCCGGAACAGCAAGGCCCTGGAAGAGAAATACGTGGCCGAAC |
| | | TGCAGCTGGAACGGCTGAAGAAAGACGGCGAAGTGCGGGGCAGCATC |
| | | AACAGATTCAAGACCAGCGACTACGTGAAAGAAGCCAAACAGCTGCTG |
| | | AAGGTGCAGAAGGCCTACCACCAGCTGGACCAGAGCTTCATCGACACC |
| | | TACATCGACCTGCTGGAAACCCGGCGGACCTACTATGAGGGACCTGGC |
| | | GAGGGCAGCCCCTTCGGCTGGAAGGACATCAAAGAATGGTACGAGATG |
| | | CTGATGGGCCACTGCACCTACTTCCCCGAGGAACTGCGGAGCGTGAAG |
| | | TACGCCTACAACGCCGACCTGTACAACGCCCTGAACGACCTGAACAAT |
| | | CTCGTGATCACCAGGGACGAGAACGAGAAGCTGGAATATTACGAGAAG |
| | | TTCCAGATCATCGAGAACGTGTTCAAGCAGAAGAAGAAGCCCACCCTG |
| | | AAGCAGATCGCCAAAGAAATCCTCGTGAACGAAGAGGATATTAAGGGC |
| | | TACAGAGTGACCAGCACCGGCAAGCCCGAGTTCACCAACCTGAAGGTG |
| | | TACCACGACATCAAGGACATTACCGCCCGGAAAGAGATTATTGAGAAC |
| | | GCCGAGCTGCTGGATCAGATTGCCAAGATCCTGACCATCTACCAGAGC |
| | | AGCGAGGACATCCAGGAAGAACTGACCAATCTGAACTCCGAGCTGACC |
| | | CAGGAAGAGATCGAGCAGATCTCTAATCTGAAGGGCTATACCGGCACC |
| | | CACAACCTGAGCCTGAAGGCCATCAACCTGATCCTGGACGAGCTGTGG |
| | | CACACCAACGACAACCAGATCGCTATCTTCAACCGGCTGAAGCTGGTG |
| | | CCCAAGAAGGTGGACCTGTCCCAGCAGAAAGAGATCCCCACCACCCTG |
| | | GTGGACGACTTCATCCTGAGCCCCGTCGTGAAGAGAAGCTTCATCCAG |
| | | AGCATCAAAGTGATCAACGCCATCATCAAGAAGTACGGCCTGCCCAAC |
| | | GACATCATTATCGAGCTGGCCCGCGAGAAGAACTCCAAGGACGCCCAG |
| | | AAAATGATCAACGAGATGCAGAAGCGGAACCGGCAGACCAACGAGCGG |
| | | ATCGAGGAAATCATCCGGACCACCGGCAAAGAGAACGCCAAGTACCTG |
| | | ATCGAGAAGATCAAGCTGCACGACATGCAGGAAGGCAAGTGCCTGTAC |
| | | AGCCTGGAAGCCATCCCTCTGGAAGATCTGCTGAACAACCCCTTCAACT |
| | | ATGAGGTGGACCACATCATCCCCAGAAGCGTGTCCTTCGACAACAGCT |
| | | TCAACAACAAGGTGCTCGTGAAGCAGGAAGAAGCCAGCAAGAAGGGCA |
| | | ACCGGACCCCATTCCAGTACCTGAGCAGCAGCGACAGCAAGATCAGCT |
| | | ACGAAACCTTCAAGAAGCACATCCTGAATCTGGCCAAGGGCAAGGGCA |
| | | GAATCAGCAAGACCAAGAAAGAGTATCTGCTGGAAGAACGGGACATCA |
| | | ACAGGTTCTCCGTGCAGAAAGACTTCATCAACCGGAACCTGGTGGATA |
| | | AAAATCAACGGGACTTTCCAAAATGTCGTAACAACTCCGCCCCATTGAC |
| | | GCAAATGGGCGGTAGGCGTGTACGGTGGGAGGTCTATATAAGCAGAGC |
| | | TCTCTGGCTAACTACCGGTGCCACCATGGCCCCAAAGAAGAAGCGGAAGGT |
| | | CGGTATCCACGGAGTCCCAGCAGCCAAGCGGAACTACATCCTGGGCCTG |
| | | GCCATCGGCATCACCAGCGTGGGCTACGGCATCATCGACTACGAGACA |
| | | CGGGACGTGATCGATGCCGGCGTGCGGCTGTTCAAAGAGGCCAACGTG |
| | | GAAAACAACGAGGGCAGGCGGAGCAAGAGAGGCGCCAGAAGGCTGAA |
| | | GCGGCGGAGGCGGCATAGAATCCAGAGAGTGAAGAAGCTGCTGTTCGA |
| | | CTACAACCTGCTGACCGACCACAGCGAGCTGAGCGGCATCAACCCCTA |
| | | CGAGGCCAGAGTGAAGGGCCTGAGCCAGAAGCTGAGCGAGGAAGAGT |
| | | TCTCTGCCGCCCTGCTGCACCTGGCCAAGAGAAGAGGCGTGCACAACG |
| | | TGAACGAGGTGGAAGAGGACACCGGCAACGAGCTGTCCACCAAAGAGC |
| | | AGATCAGCCGGAACAGCAAGGCCCTGGAAGAGAAATACGTGGCCGAAC |
| | | TGCAGCTGGAACGGCTGAAGAAAGACGGCGAAGTGCGGGGCAGCATC |
| | | AACAGATTCAAGACCAGCGACTACGTGAAAGAAGCCAAACAGCTGCTG |
| | | AAGGTGCAGAAGGCCTACCACCAGCTGGACCAGAGCTTCATCGACACC |
| | | TACATCGACCTGCTGGAAACCCGGCGGACCTACTATGAGGGACCTGGC |
| | | GAGGGCAGCCCCTTCGGCTGGAAGGACATCAAAGAATGGTACGAGATG |
| | | CTGATGGGCCACTGCACCTACTTCCCCGAGGAACTGCGGAGCGTGAAG |
| | | TACGCCTACAACGCCGACCTGTACAACGCCCTGAACGACCTGAACAAT |
| | | CTCGTGATCACCAGGGACGAGAACGAGAAGCTGGAATATTACGAGAAG |
| | | TTCCAGATCATCGAGAACGTGTTCAAGCAGAAGAAGAAGCCCACCCTG |
| | | AAGCAGATCGCCAAAGAAATCCTCGTGAACGAAGAGGATATTAAGGGC |
| | | TACAGAGTGACCAGCACCGGCAAGCCCGAGTTCACCAACCTGAAGGTG |
| | | TACCACGACATCAAGGACATTACCGCCCGGAAAGAGATTATTGAGAAC |
| | | ATCAAAAAGGGCAAAAGGCCGGCGGCCACGAAAAAGGCCGGCCAGGCAA |
| | | AAAAGAAAAAGGGATCCGAGGCCAGCGGTTCCGGACGGGCTGACGCATTG |
| | | GACGATTTTGATCTGGATATGCTGGGAAGTGACGCCCTCGATGATTTTG |
| | | ACCTTGACATGCTTGGTTCGGATGCCCTTGATGACTTTGACCTCGACAT |
| | | GCTCGGCAGTGACGCCCTTGATGATTTCGACCTGGACATGCTGTGATCT |
| | | AGAGAATTCCTAGAGCTCGCTGATCAGCCTCGACTGTGCCTTCTAGTTGCCA |
| | | GCCATCTGTTGTTTGCCCCTCCCCCGTGCCTTCCTTGACCCTGGAAGGTGCC |
| | | ACTCCCACTGTCCTTTCCTAATAAAATGAGGAAATTGCATCGCATTGTCTGA |
| | | GTAGGTGTCATTCTATTCTGGGGGGTGGGGGGGGCAGGACAGCAAGGGGG |
| | | AGGATTGGGAAGAGAATAGCAGGCATGCTGGGGAgctagaggccgcaggaacccctagt |
| | | gatggagttggccactccctctctgcgcgctcgctcgctcactgaggccgggcgaccaaaggtcgcccgacgcccggg |
| | | cggcctcagtgagcgagcgagcgcgcagctgcctgcaggacatgtgagcaaaaggccagcaaaaggccaggaaccgt |
| | | aaaaaggccgcgttgctggcgtttttccataggctccgcccccctgacgagcatcacaaaaatcgacgctcaagtcagaggtg |
| | | gcgaaacccgacaggactataaagataccaggcgtttccccctggaagctccctcgtgcgctctcctgttccgaccctgccgc |
| | | ttaccggatacctgtccgcctttctcccttcgggaagcgtggcgctttctcatagctcacgctgtaggtatctcagttcgctgtag |
| | | gtcgttcgctccaagctgggctgtgtgcacgaaccccccgttcagcccgaccgctgcgccttatccggtaactatcgtcttgag |
| | | tccaacccggtaagacacgacttatcgccactggcagcagccactggtaacaggattagcagagcgaggtatgtaggcggt |
| | | gctacagagttcttgaagtggtggcctaactacggctacactagaagaacagtatttggtatctgcgctctgctgaagccagtta |
| | | ccttcggaaaaagagttggtagctcttgatccggcaaacaaaccaccgctggtagcggtggtttttttgtttgcaagcagcagat |
| | | tacgcgcagaaaaaaaggatctcaagaagatcctttgatcttttctacggggtctgacgctcagtggaacgaaaactcacgtta |
| | | agggattttggtcatgagattatcaaaaaggatcttcacctagatccttttaaattaaaaatgaagttttaaatcaatctaaagtatat |
| | | atgagtaaacttggtctgacagttaccaatgcttaatcagtgaggcacctatctcagcgatctgtctatttcgttcatccata |
| | | gttgcctgactccccgtcgtgtagataactacgatacgggagggcttaccatctggccccagtgctgcaatgataccgc |
| | | ggcttccacgctcaccggctccagatttatcagcaataaaccagccagccggaagggccgagcgcagaagtggtcct |
| | | gcaactttatccgcctccatccagtctattaattgttgccgggaagctagagtaagtagttcgccagttaatagtttgcgc |
| | | aacgttgttgccattgctacaggcatcgtggtgtcacgctcgtcgtttggtatggcttcattcagctccggttcccaacga |
| | | tcaaggcgagttacatgatcccccatgttgtgcaaaaaagcggttagctccttcggtcctccgatcgttgtcagaagtaa |
| | | gttggccgcagtgttatcactcatggttatgg |
| 9 | AAV-U6- | cagcactgcataattctcttactgtcatgccatccgtaagatgcttttctgtgactggtgagtactcaaccaagtcattctg |
| | Shank3- | agaatagtgtatgcggcgaccgagttgctcttgcccggcgtcaatacgggataataccgcgccacatagcagaacttt |
| | gRNA1-CMV- | aaaagtgctcatcattggaaaacgttcttcggggcgaaaactctcaaggatcttaccgctgttgagatccagttcgatgt |
| | dSaCas9-VP64 | aacccactcgtgcacccaactgatcttcagcatcttttactttcaccagcgtttctgggtgagcaaaaacaggaaggca |
| | 5′ part of | aaatgccgcaaaaaagggaataagggcgacacggaaatgttgaatactcatactcttcctttttaatattattgaagcattt |
| | Ampicillin | atcagggttattgtctcatgagcggatacatatttgaatgtatttagaaaaataaacaaataggggttccgcgcacatttccccga |
| | resistant gene | aaagtgccacctgacgtctaagaaaccattattatcatgacattaacctataaaaataggcgtatcacgaggccctttcgtctcgc |
| | (AmpR) (bold)- | gcgtttcggtgatgacggtgaaaacctctgacacatgcagctcccggagacggtcacagcttgtctgtaagcggatgccggg |
| | AmpR promoter | agcagacaagcccgtcagggcgcgtcagcgggtgttggcgggtgtcggggctggcttaactatgcggcatcagagcagatt |
| | (underlined)-f1 | gtactgagagtgcaccataaaattgtaaacgttaatattttgttaaaattcgcgttaaatttttgttaaatcagctcatttttt |
| | bacteriophage | aaccaatagaccgaaatcggcaaaatcccttataaatcaaaagaatagcccgagatagagttgagtgttgttccagtt |
| | origin of | tggaacaagagtccactattaaagaacgtggactccaacgtcaaagggcgaaaaaccgtctatcagggcgatggcc |
| | replication | cactacgtgaaccatcacccaaatcaagttttttggggtcgaggtgccgtaaagcactaaatcggaaccctaaagggagc |
| | (bold)-5′ITR | ccccgatttagagcttgacggggaaagccggcgaacgtggcgagaaaggaagggaagaaagcgaaaggagcgggcgct |
| | (underlined)- | aaggcgctggcaagtgtagcggtcacgctgcgcgtaaccaccacacccgccgcgcttaatgcgccgctacagggcgcgta |
| | U6 promoter | ctatggttgctttgacgtatgcggtgtgaaataccgcacagatgcgtaaggagaaaataccgcatcaggcgcccctgcaggca |
| | (bold)-Shank3 | gctgcgcgctcgctcgctcactgaggccgccccgccaaagcccgggcgtcgggcgacctttggtcgcccggcctcagtga |
| | gRNA1 | gcgagcgagcgcgcagagagggagtggccaactccatcactaggggttcctgcggccgcctcgagGAGGGCCT |
| | underlined)- | ATTTCCCATGATTCCTTCATATTTGCATATACGATACAAGGCTGTTAGA |
| | gRNA scaffold | GAGATAATTGGAATTAATTTGACTGTAAACACAAAGATATTAGTACAAA |
| | (bold)-CMV | ATACGTGACGTAGAAAGTAATAATTTCTTGGGTAGTTTGCAGTTTTAAA |
| | promoter | ATTATGTTTTAAAATGGACTATCATATGCTTACCGTAACTTGAAAGTAT |
| | (underlined)- | TTCGATTTCTTGGCTTTATATATCTTGTGGAAAGGACGAAACACCGGCG |
| | SV40 nuclear | GTCGGCGGGGCTCTTGCGGTTATAGTACTCTGGAAACAGAATCTACTATA |
| | localization | ACAAGGCAAAATGCCGTGTTTATCTCGTCAACTTGTTGGCGAGATTTTT |
| | signal (bold)- | TTGCGTTGACATTGATTATTGACTAGTTATTAATAGTAATCAATTACGGGGT |
| | dSaCas9 | CATTAGTTCATAGCCCATATATGGAGTTCCGCGTTACATAACTTACGGTAAA |
| | (underlined)- | TGGCCCGCCTGGCTGACCGCCCAACGACCCCCGCCCATTGACGTCAATAAT |
| | Nucleoplasmin | GACGTATGTTCCCATAGTAACGCCAATAGGGACTTTCCATTGACGTCAATGG |
| | nuclear | GTGGAGTATTTACGGTAAACTGCCCACTTGGCAGTACATCAAGTGTATCATA |
| | localization | TGCCAAGTACGCCCCCTATTGACGTCAATGACGGTAAATGGCCCGCCTGGC |
| | signal (bold)- | ATTATGCCCAGTACATGACCTTATGGGACTTTCCTACTTGGCAGTACATCTA |
| | VP64 | CGTATTAGTCATCGCTATTACCATGGTGATGCGGTTTTGGCAGTACATCAAT |
| | (underlined)- | GGGCGTGGATAGCGGTTTGACTCACGGGGATTTCCAAGTCTCCACCCCATTG |
| | Bovine growth | ACGTCAATGGGAGTTTGTTTTGGCACCAAAATCAACGGGACTTTCCAAAAT |
| | hormone | GTCGTAACAACTCCGCCCCATTGACGCAAATGGGCGGTAGGCGTGTACGGT |
| | polyadenylation | GGGAGGTCTATATAAGCAGAGCTCTCTGGCTAACTACCGGTGCCACCATGG |
| | signal (bold)- | CCCCAAAGAAGAAGCGGAAGGTCGGTATCCACGGAGTCCCAGCAGCCAA |
| | 3′ITR | GCGGAACTACATCCTGGGCCTGGCCATCGGCATCACCAGCGTGGGCTACGG |
| | (underlined)- | CATCATCGACTACGAGACACGGGACGTGATCGATGCCGGCGTGCGGCTGTT |
| | high-copy- | CAAAGAGGCCAACGTGGAAAACAACGAGGGCAGGCGGAGCAAGAGAGGC |
| | number | GCCAGAAGGCTGAAGCGCCGGAGGCGGCATAGAATCCAGAGAGTGAAGAA |
| | ColEI/pMB1/ | GCTGCTGTTCGACTACAACCTGCTGACCGACCACAGCGAGCTGAGCGGCAT |
| | pBR322/pUC | CAACCCCTACGAGGCCAGAGTGAAGGGCCTGAGCCAGAAGCTGAGCGAGG |
| | origin of | AAGAGTTCTCTGCCGCCCTGCTGCACCTGGCCAAGAGAAGAGGCGTGCACA |
| | replication | ACGTGAACGAGGTGGAAGAGGACACCGGCAACGAGCTGTCCACCAAAGAG |
| | (bold)-3′ part | CAGATCAGCCGGAACAGCAAGGCCCTGGAAGAGAAATACGTGGCCGAACT |
| | of Ampicillin | GCAGCTGGAACGGCTGAAGAAAGACGGCGAAGTGCGGGGCAGCATCAACA |
| | resistant gene | GATTCAAGACCAGCGACTACGTGAAAGAAGCCAAACAGCTGCTGAAGGTG |
| | (AmpR) | CAGAAGGCCTACCACCAGCTGGACCAGAGCTTCATCGACACCTACATCGAC |
| | (underlined) | CTGCTGGAAACCCGGCGGACCTACTATGAGGGACCTGGCGAGGGCAGCCCC |
| | | TTCGGCTGGAAGGACATCAAAGAATGGTACGAGATGCTGATGGGCCACTGC |
| | | ACCTACTTCCCCGAGGAACTGCGGAGCGTGAAGTACGCCTACAACGCCGAC |
| | | CTGTACAACGCCCTGAACGACCTGAACAATCTCGTGATCACCAGGGACGAG |
| | | AACGAGAAGCTGGAATATTACGAGAAGTTCCAGATCATCGAGAACGTGTTC |
| | | AAGCAGAAGAAGAAGCCCACCCTGAAGCAGATCGCCAAAGAAATCCTCGT |
| | | GAACGAAGAGGATATTAAGGGCTACAGAGTGACCAGCACCGGCAAGCCCG |
| | | AGTTCACCAACCTGAAGGTGTACCACGACATCAAGGACATTACCGCCCGGA |
| | | AAGAGATTATTGAGAACGCCGAGCTGCTGGATCAGATTGCCAAGATCCTGA |
| | | CCATCTACCAGAGCAGCGAGGACATCCAGGAAGAACTGACCAATCTGAACT |
| | | CCGAGCTGACCCAGGAAGAGATCGAGCAGATCTCTAATCTGAAGGGCTATA |
| | | CCGGCACCCACAACCTGAGCCTGAAGGCCATCAACCTGATCCTGGACGAGC |
| | | TGTGGCACACCAACGACAACCAGATCGCTATCTTCAACCGGCTGAAGCTGG |
| | | TGCCCAAGAAGGTGGACCTGTCCCAGCAGAAAGAGATCCCCACCACCCTGG |
| | | TGGACGACTTCATCCTGAGCCCCGTCGTGAAGAGAAGCTTCATCCAGAGCA |
| | | TCAAAGTGATCAACGCCATCATCAAGAAGTACGGCCTGCCCAACGACATCA |
| | | TTATCGAGCTGGCCCGCGAGAAGAACTCCAAGGACGCCCAGAAAATGATCA |
| | | ACGAGATGCAGAAGCGGAACCGGCAGACCAACGAGCGGATCGAGGAAATC |
| | | ATCCGGACCACCGGCAAAGAGAACGCCAAGTACCTGATCGAGAAGATCAA |
| | | GCTGCACGACATGCAGGAAGGCAAGTGCCTGTACAGCCTGGAAGCCATCCC |
| | | TCTGGAAGATCTGCTGAACAACCCCTTCAACTATGAGGTGGACCACATCAT |
| | | CCCCAGAAGCGTGTCCTTCGACAACAGCTTCAACAACAAGGTGCTCGTGAA |
| | | GCAGGAAGAAGCCAGCAAGAAGGGCAACCGGACCCCATTCCAGTACCTGA |
| | | GCAGCAGCGACAGCAAGATCAGCTACGAAACCTTCAAGAAGCACATCCTGA |
| | | ATCTGGCCAAGGGCAAGGGCAGAATCAGCAAGACCAAGAAAGAGTATCTG |
| | | CTGGAAGAACGGGACATCAACAGGTTCTCCGTGCAGAAAGACTTCATCAAC |
| | | CGGAACCTGGTGGATACCAGATACGCCACCAGAGGCCTGATGAACCTGCTG |
| | | CGGAGCTACTTCAGAGTGAACAACCTGGACGTGAAAGTGAAGTCCATCAAT |
| | | GGCGGCTTCACCAGCTTTCTGCGGCGGAAGTGGAAGTTTAAGAAAGAGCGG |
| | | AACAAGGGGTACAAGCACCACGCCGAGGACGCCCTGATCATTGCCAACGCC |
| | | GATTTCATCTTCAAAGAGTGGAAGAAACTGGACAAGGCCAAAAAAGTGATG |
| | | GAAAACCAGATGTTCGAGGAAAAGCAGGCCGAGAGCATGCCCGAGATCGA |
| | | AACCGAGCAGGAGTACAAAGAGATCTTCATCACCCCCCACCAGATCAAGCA |
| | | CATTAAGGACTTCAAGGACTACAAGTACAGCCACCGGGTGGACAAGAAGCC |
| | | TAATAGAGAGCTGATTAACGACACCCTGTACTCCACCCGGAAGGACGACAA |
| | | GGGCAACACCCTGATCGTGAACAATCTGAACGGCCTGTACGACAAGGACAA |
| | | TGACAAGCTGAAAAAGCTGATCAACAAGAGCCCCGAAAAGCTGCTGATGTA |
| | | CCACCACGACCCCCAGACCTACCAGAAACTGAAGCTGATTATGGAACAGTA |
| | | CGGCGACGAGAAGAATCCCCTGTACAAGTACTACGAGGAAACCGGGAACT |
| | | ACCTGACCAAGTACTCCAAAAAGGACAACGGCCCCGTGATCAAGAAGATTA |
| | | AGTATTACGGCAACAAACTGAACGCCCATCTGGACATCACCGACGACTACC |
| | | CCAACAGCAGAAACAAGGTCGTGAAGCTGTCCCTGAAGCCCTACAGATTCG |
| | | ACGTGTACCTGGACAATGGCGTGTACAAGTTCGTGACCGTGAAGAATCTGG |
| | | ATGTGATCAAAAAAGAAAACTACTACGAAGTGAATAGCAAGTGCTATGAG |
| | | GAAGCTAAGAAGCTGAAGAAGATCAGCAACCAGGCCGAGTTTATCGCCTCC |
| | | TTCTACAACAACGATCTGATCAAGATCAACGGCGAGCTGTATAGAGTGATC |
| | | GGCGTGAACAACGACCTGCTGAACCGGATCGAAGTGAACATGATCGACATC |
| | | ACCTACCGCGAGTACCTGGAAAACATGAACGACAAGAGGCCCCCCAGGATC |
| | | ATTAAGACAATCGCCTCCAAGACCCAGAGCATTAAGAAGTACAGCACAGAC |
| | | ATTCTGGGCAACCTGTATGAAGTGAAATCTAAGAAGCACCCTCAGATCATC |
| | | AAAAAGGGCAAAAGGCCGGCGGCCACGAAAAAGGCCGGCCAGGCAAAA |
| | | AAGAAAAAGGGATCCGAGGCCAGCGGTTCCGGACGGGCTGACGCATTGGA |
| | | CGATTTTGATCTGGATATGCTGGGAAGTGACGCCCTCGATGATTTTGACCTT |
| | | GACATGCTTGGTTCGGATGCCCTTGATGACTTTGACCTCGACATGCTCGGCA |
| | | GTGACGCCCTTGATGATTTCGACCTGGACATGCTGTGATCTAGAGAATTCCT |
| | | AGAGCTCGCTGATCAGCCTCGACTGTGCCTTCTAGTTGCCAGCCATCTGT |
| | | TGTTTGCCCCTCCCCCGTGCCTTCCTTGACCCTGGAAGGTGCCACTCCC |
| | | ACTGTCCTTTCCTAATAAAATGAGGAAATTGCATCGCATTGTCTGAGTA |
| | | GGTGTCATTCTATTCTGGGGGGTGGGGGGGGCAGGACAGCAAGGGG |
| | | GAGGATTGGGAAGAGAATAGCAGGCATGCTGGGGAgctagaggccgcaggaaccc |
| | | ctagtgatggagttggccactccctctctgcgcgctcgctcgctcactgaggccgggcgaccaaaggtcgcccgacgcccg |
| | | ggggcctcagtgagcgagcgagcgcgcagctgcctgcaggacatgtgagcaaaaggccagcaaaaggccaggaaccg |
| | | taaaaaggccgcgttgctggcgtttttccataggctccgcccccctgacgagcatcacaaaaatcgacgctcaagtcaga |
| | | ggtggcgaaacccgacaggactataaagataccaggcgtttccccctggaagctccctcgtgcgctctcctgttccgac |
| | | cctgccgcttaccggatacctgtccgcctttctcccttcgggaagcgtggcgctttctcatagctcacgctgtaggtatctc |
| | | agttcggtgtaggtcgttcgctccaagctgggctgtgtgcacgaaccccccgttcagcccgaccgctgcgccttatccggt |
| | | aactatcgtcttgagtccaacccggtaagacacgacttatcgccactggcagcagccactggtaacaggattagcagagcga |
| | | ggtatgtaggggtgctacagagttcttgaagtggtggcctaactacggctacactagaagaacagtatttggtatctgcgctct |
| | | gctgaagccagttaccttcggaaaaagagttggtagctcttgatccggcaaacaaaccaccgctggtagcggtggtttttttttt |
| | | gcaagcagcagattacgcgcagaaaaaaaggatctcaagaagatcctttgatctttctacggggtctgacgctcagtggaac |
| | | gaaaactcacgttaagggattttggtcatgagattatcaaaaaggatcttcacctagatccttttaaattaaaaatgaagttttaaat |
| | | caatctaaagtatatatgagtaaacttggtctgacagttaccaatgcttaatcagtgaggcacctatctcagcgatctgtctatttcg |
| | | ttcatccatagttgcctgactccccgtcgtgtagataactacgatacgggagggcttaccatctggccccagtgctgcaatgata |
| | | ccgcggcttccacgctcaccggctccagatttatcagcaataaaccagccagccggaagggccgagcgcagaagtggtcct |
| | | gcaactttatccgcctccatccagtcattaattgttgccgggaagctagagtaagtagttcgccagttaatagtttgcgcaacgtt |
| | | gttgccattgctacaggcatctggtgtcacgctcgtcgtttggtatggcttcattcagctccggttcccaacgatcaaggcgagt |
| | | tacatgatcccccatcttctccaaaaaagccgttagctccttccgtcctccgatccttctcagaagtaagttcgccgcactcttat |
| | | cactcatggttatgg |
| 10 | AAV-U6- | cagcactgcataattctcttactgtcatgccatccgtaagatgcttttctgtgactggtgagtactcaaccaagtcattctg |
| | Shank3- | agaatagtgtatgcggcgaccgagttgctcttgcccggcgtcaatacgggataataccgcgccacatagcagaacttt |
| | gRNA2-CMV- | aaaagtgctcatcattggaaaacgttcttcggggcgaaaactctcaaggatcttaccgctgttgagatccagttcgatgt |
| | dSaCas9-VP64 | aacccactcgtgcacccaactgatcttcagcatcttttactttcaccagcgtttctgggtgagcaaaaacaggaaggca |
| | 5′ part of | aaatgccgcaaaaaagggaataagggcgacacggaaatgttgaatactcatactcttcctttttcaatattattgaagcattt |
| | Ampicillin | atcagggttattgtctcatgagcggatacatatttgaatgtatttagaaaaataaacaaataggggttccgcgcacatttccccga |
| | resistant gene | aaagtgccacctgacgtctaagaaaccattattatcatgacattaacctataaaaataggcgtatcacgaggccctttcgtctcgc |
| | (AmpR) (bold)- | gcgtttcggtgatgacggtgaaaacctctgacacatgcagctcccggagacggtcacagcttgtctgtaagcggatgccggg |
| | AmpR promoter | agcagacaagcccgtcagggcgcgtcaggggtgttggcgggtgtcggggctggcttaactatgcggcatcagagcagatt |
| | (underlined)-f1 | gtactgagagtgcaccataaaattgtaaacgttaatattttgttaaaattcgcgttaaatttttgttaaatcagctcatttttt |
| | bacteriophage | aaccaatagaccgaaatcggcaaaacccttataaatcaaaagaatagcccgagatagagttgagtgttgttccagtt |
| | origin of | tggaacaagagtccactattaaagaacgtggactccaacgtcaaagggcgaaaaaccgtctatcagggcgatggcc |
| | replication | cactacgtgaaccatcacccaaatcaagttttttggggtcgaggtgccgtaaagcactaaatcggaaccctaaagggagc |
| | (bold)-5′ITR | ccccgatttagagcttgacggggaaagccggcgaacgtggcgagaaaggaagggaagaaagcgaaaggagcgggcgct |
| | (underlined)- | aaggcgctggcaagtgtagcggtcacgctgcgcgtaaccaccacacccgccgcgcttaatgcgccgctacagggcgcgta |
| | U6 promoter | ctatggttgctttgacgtatgcggtgtgaaataccgcacagatgcgtaaggagaaaataccgcatcaggcgcccctgcaggca |
| | (bold)-Shank3 | gctgcgcgctcgctcgctcactgaggccgcccgggcaaagcccggggtcgggcgacctttcgtcgcccggcctcatga |
| | gRNA2 | gcgagcgagcgcccagagagggagtcgccaactccatcactaggggttcctgcggccgcctcgagGAGGGCCT |
| | (underlined)- | ATTTCCCATGATTCCTTCATATTTGCATATACGATACAAGGCTGTTAGA |
| | gRNA scaffold | GAGATAATTGGAATTAATTTGACTGTAAACACAAAGATATTAGTACAAA |
| | (bold)-CMV | ATACGTGACGTAGAAAGTAATAATTTCTTGGGTAGTTTGCAGTTTTAAA |
| | promoter | ATTATGTTTTAAAATGGACTATCATATGCTTACCGTAACTTGAAAGTAT |
| | (underlined)- | TTCGATTTCTTGGCTTTATATATCTTGTGGAAAGGACGAAACACCGGGA |
| | SV40 nuclear | GGGGGCCGCGCCGGAGGCGTTATAGTACTCTGGAAACAGAATCTACTAT |
| | localization | AACAAGGCAAAATGCCGTGTTTATCTCGTCAACTTGTTGGCGAGATTTT |
| | signal (bold)- | TTTGCGTTGACATTGATTATTGACTAGTTATTAATAGTAATCAATTACGGGG |
| | dSaCas9 | TCATTAGTTCATAGCCCATATATGGAGTTCCGCGTTACATAACTTACGGTAA |
| | (underlined)- | ATGGCCCGCCTGGCTGACCGCCCAACGACCCCCGCCCATTGACGTCAATAA |
| | Nucleoplasmin | TGACGTATGTTCCCATAGTAACGCCAATAGGGACTTTCCATTGACGTCAATG |
| | nuclear | GGTGGAGTATTTACGGTAAACTGCCCACTTGGCAGTACATCAAGTGTATCAT |
| | localization | ATGCCAAGTACGCCCCCTATTGACGTCAATGACGGTAAATGGCCCGCCTGG |
| | signal (bold)- | CATTATGCCCAGTACATGACCTTATGGGACTTTCCTACTTGGCAGTACATCT |
| | VP64 | ACGTATTAGTCATCGCTATTACCATGGTGATGCGGTTTTGGCAGTACATCAA |
| | (underlined)- | TGGGCGTGGATAGCGGTTTGACTCACGGGGATTTCCAAGTCTCCACCCCATT |
| | Bovine growth | GACGTCAATGGGAGTTTGTTTTGGCACCAAAATCAACGGGACTTTCCAAAA |
| | hormone | TGTCGTAACAACTCCGCCCCATTGACGCAAATGGGCGGTAGGCGTGTACGG |
| | polyadenylation | TGGGAGGTCTATATAAGCAGAGCTCTCTGGCTAACTACCGGTGCCACCATG |
| | signal (bold)- | GCCCCAAAGAAGAAGCGGAAGGTCGGTATCCACGGAGTCCCAGCAGCCA |
| | 3′ITR | AGCGGAACTACATCCTGGGCCTGGCCATCGGCATCACCAGCGTGGGCTACG |
| | (underlined)- | GCATCATCGACTACGAGACACGGGACGTGATCGATGCCGGCGTGCGGCTGT |
| | high-copy- | TCAAAGAGGCCAACGTGGAAAACAACGAGGGCAGGCGGAGCAAGAGAGGC |
| | number | GCCAGAAGGCTGAAGCGGCGGAGGCGGCATAGAATCCAGAGAGTGAAGAA |
| | ColE1/pMB1/ | GCTGCTGTTCGACTACAACCTGCTGACCGACCACAGCGAGCTGAGCGGCAT |
| | pBR322/pUC | CAACCCCTACGAGGCCAGAGTGAAGGGCCTGAGCCAGAAGCTGAGCGAGG |
| | origin of | AAGAGTTCTCTGCCGCCCTGCTGCACCTGGCCAAGAGAAGAGGCGTGCACA |
| | replication | ACGTGAACGAGGTGGAAGAGGACACCGGCAACGAGCTGTCCACCAAAGAG |
| | (bold)-3′ part | CAGATCAGCCGGAACAGCAAGGCCCTGGAAGAGAAATACGTGGCCGAACT |
| | of Ampicillin | GCAGCTGGAACGGCTGAAGAAAGACGGCGAAGTGCGGGGCAGCATCAACA |
| | resistant gene | GATTCAAGACCAGCGACTACGTGAAAGAAGCCAAACAGCTGCTGAAGGTg |
| | AmpR) | CAGAAGGCCTACCACCAGCTGGACCAGAGCTTCATCGACACCTACATCGAC |
| | (underlined) | CTGCTGGAAACCCGGCGGACCTACTATGAGGGACCTGGCGAGGGCAGCCCC |
| | | TTCGGCTGGAAGGACATCAAAGAATGGTACGAGATGCTGATGGGCCACTGC |
| | | ACCTACTTCCCCGAGGAACTGCGGAGCGTGAAGTACGCCTACAACGCCGAC |
| | | CTGTACAACGCCCTGAACGACCTGAACAATCTCGTGATCACCAGGGACGAG |
| | | AACGAGAAGCTGGAATATTACGAGAAGTTCCAGATCATCGAGAACGTGTTC |
| | | AAGCAGAAGAAGAAGCCCACCCTGAAGCAGATCGCCAAAGAAATCCTCGT |
| | | GAACGAAGAGGATATTAAGGGCTACAGAGTGACCAGCACCGGCAAGCCCG |
| | | AGTTCACCAACCTGAAGGTGTACCACGACATCAAGGACATTACCGCCCGGA |
| | | AAGAGATTATTGAGAACGCCGAGCTGCTGGATCAGATTGCCAAGATCCTGA |
| | | CCATCTACCAGAGCAGCGAGGACATCCAGGAAGAACTGACCAATCTGAACT |
| | | CCGAGCTGACCCAGGAAGAGATCGAGCAGATCTCTAATCTGAAGGGCTATA |
| | | CCGGCACCCACAACCTGAGCCTGAAGGCCATCAACCTGATCCTGGACGAGC |
| | | TGTGGCACACCAACGACAACCAGATCGCTATCTTCAACCGGCTGAAGCTGG |
| | | TGCCCAAGAAGGTGGACCTGTCCCAGCAGAAAGAGATCCCCACCACCCTGG |
| | | TGGACGACTTCATCCTGAGCCCCGTCGTGAAGAGAAGCTTCATCCAGAGCA |
| | | TCAAAGTGATCAACGCCATCATCAAGAAGTACGGCCTGCCCAACGACATCA |
| | | TTATCGAGCTGGCCCGCGAGAAGAACTCCAAGGACGCCCAGAAAATGATCA |
| | | ACGAGATGCAGAAGCGGAACCGGCAGACCAACGAGCGGATCGAGGAAATC |
| | | ATCCGGACCACCGGCAAAGAGAACGCCAAGTACCTGATCGAGAAGATCAA |
| | | GCTGCACGACATGCAGGAAGGCAAGTGCCTGTACAGCCTGGAAGCCATCCC |
| | | TCTGGAAGATCTGCTGAACAACCCCTTCAACTATGAGGTGGACCACATCAT |
| | | CCCCAGAAGCGTGTCCTTCGACAACAGCTTCAACAACAAGGTGCTCGTGAA |
| | | GCAGGAAGAAGCCAGCAAGAAGGGCAACCGGACCCCATTCCAGTACCTGA |
| | | GCAGCAGCGACAGCAAGATCAGCTACGAAACCTTCAAGAAGCACATCCTGA |
| | | ATCTGGCCAAGGGCAAGGGCAGAATCAGCAAGACCAAGAAAGAGTATCTG |
| | | CTGGAAGAACGGGACATCAACAGGTTCTCCGTGCAGAAAGACTTCATCAAC |
| | | CGGAACCTGGTGGATACCAGATACGCCACCAGAGGCCTGATGAACCTGCTG |
| | | CGGAGCTACTTCAGAGTGAACAACCTGGACGTGAAAGTGAAGTCCATCAAT |
| | | GGCGGCTTCACCAGCTTTCTGCGGCGGAAGTGGAAGTTTAAGAAAGAGCGG |
| | | AACAAGGGGTACAAGCACCACGCCGAGGACGCCCTGATCATTGCCAACGCC |
| | | GATTTCATCTTCAAAGAGTGGAAGAAACTGGACAAGGCCAAAAAAGTGATG |
| | | GAAAACCAGATGTTCGAGGAAAAGCAGGCCGAGAGCATGCCCGAGATCGA |
| | | AACCGAGCAGGAGTACAAAGAGATCTTCATCACCCCCCACCAGATCAAGCA |
| | | CATTAAGGACTTCAAGGACTACAAGTACAGCCACCGGGTGGACAAGAAGCC |
| | | TAATAGAGAGCTGATTAACGACACCCTGTACTCCACCCGGAAGGACGACAA |
| | | GGGCAACACCCTGATCGTGAACAATCTGAACGGCCTGTACGACAAGGACAA |
| | | TGACAAGCTGAAAAAGCTGATCAACAAGAGCCCCGAAAAGCTGCTGATGTA |
| | | CCACCACGACCCCCAGACCTACCAGAAACTGAAGCTGATTATGGAACAGTA |
| | | CGGCGACGAGAAGAATCCCCTGTACAAGTACTACGAGGAAACCGGGAACT |
| | | ACCTGACCAAGTACTCCAAAAAGGACAACGGCCCCGTGATCAAGAAGATTA |
| | | AGTATTACGGCAACAAACTGAACGCCCATCTGGACATCACCGACGACTACC |
| | | CCAACAGCAGAAACAAGGTCGTGAAGCTGTCCCTGAAGCCCTACAGATTCG |
| | | ACGTGTACCTGGACAATGGCGTGTACAAGTTCGTGACCGTGAAGAATCTGG |
| | | ATGTGATCAAAAAAGAAAACTACTACGAAGTGAATAGCAAGTGCTATGAG |
| | | GAAGCTAAGAAGCTGAAGAAGATCAGCAACCAGGCCGAGTTTATCGCCTCC |
| | | TTCTACAACAACGATCTGATCAAGATCAACGGCGAGCTGTATAGAGTGATG |
| | | GGCGTGAACAACGACCTGCTGAACCGGATCGAAGTGAACATGATCGACATC |
| | | ACCTACCGCGAGTACCTGGAAAACATGAACGACAAGAGGCCCCCCAGGATC |
| | | ATTAAGACAATCGCCTCCAAGACCCAGAGCATTAAGAAGTACAGCACAGAC |
| | | ATTCTGGGCAACCTGTATGAAGTGAAATCTAAGAAGCACCCTCAGATCATC |
| | | AAAAAGGGCAAAAGGCCGGCGGCCACGAAAAAGGCCGGCCAGGCAAAA |
| | | AAGAAAAAGGGATCCGAGGCCAGCGGTTCCGGACGGGCTGACGCATTGGA |
| | | CGATTTTGATCTGGATATGCTGGGAAGTGACGCCCTCGATGATTTTGACCTT |
| | | GACATGCTTGGTTCGGATGCCCTTGATGACTTTGACCTCGACATGCTCGGCA |
| | | GTGACGCCCTTGATGATTTCGACCTGGACATGCTGTGATCTAGAGAATTCCT |
| | | AGAGCTCGCTGATCAGCCTCGACTGTGCCTTCTAGTTGCCAGCCATCTGT |
| | | TGTTTGCCCCTCCCCCGTGCCTTCCTTGACCCTGGAAGGTGCCACTCCC |
| | | ACTGTCCTTTCCTAATAAAATGAGGAAATTGCATCGCATTGTCTGAGTA |
| | | GGTGTCATTCTATTCTGGGGGGTGGGGGGGGCAGGACAGCAAGGGG |
| | | GAGGATTGGGAAGAGAATAGCAGGCATGCTGGGGAgctagaggccgcaggaaccc |
| | | ctagtgatggagttcgccactccctctctgcgcgctcgctcgctcactgaggccggccgaccaaaggtcgcccgacgcccg |
| | | ggcggcctcagtgagcgagcgagcgcgcagctgcctgcaggacatgtgagcaaaaggccagcaaaaggccaggaaccg |
| | | taaaaaggccgcgttgctggcgtttttccataggctccgcccccctgacgagcatcacaaaaatcgacgctcaagtcaga |
| | | ggtggcgaaacccgacaggactataaagataccaggcgtttccccctggaagctccctcgtgcgctctcctgttccgac |
| | | cctgccgcttaccggatacctgtccgcctttctcccttcgggaagcgtggcgctttctcatagctcacgctgtaggtatctc |
| | | agttcggtgtaggtcgttcgctccaagctgggctgtgtgcacgaaccccccgttcagcccgaccgctgcgccttatccggt |
| | | aactatcgtcttgagtccaacccggtaagacacgacttatcgccactggcagcagccactggtaacaggattagcagagcga |
| | | ggtatgtaggcggtgctacagagttcttgaagtggtggcctaactacggctacactagaagaacagtatttggtatctgcgctct |
| | | gctgaagccagttaccttcggaaaaagagttggtagctcttgatccggcaaacaaaccaccgctggtagcggtggtttttttgttt |
| | | gcaagcagcagattacgcgcagaaaaaaaggatctcaagaagatcctttgatcttttctacggggtctgacgctcagtggaac |
| | | gaaaactcacgttaagggattttggtcatgagattatcaaaaaggatcttcacctagatccttttaaattaaaaatgaagttttaaat |
| | | caatctaaagtatatatgagtaaacttggtctgacagttaccaatgcttaatcagtgaggcacctatctcagcgatctctctatttcg |
| | | ttcatccatagttgcctgactccccgtcgtgtagataactacgatacgggagggcttaccatctcgccccagtgctgcaatgata |
| | | ccgcggcttccacgctcaccggctccagatttatcagcaataaaccagccagccggaagggccgagcgcagaagtggtcct |
| | | gcaactttatccgcctccatccagtctattaattcttgccgggaagctagagtaagtagttccccagttaatagtttgcgcaacgtt |
| | | gttgccattgctacaggcatcgtggtgtcacgctcgtcgtttggtatggcttcattcagctccggttcccaacgatcaaggcgagt |
| | | tacatgatcccccatgttgtgcaaaaaagcggttagctccttcggtcctccgatcgttgtcagaagtaagttggccgcagtgttat |
| | | cactcatggttatgg |
| 11 | AAV-U6- | cagcactgcataattctcttactgtcatgccatccgtaagatgcttttctgtgactggtgagtactcaaccaagtcattctg |
| | Shank3- | agaatagtgtatgcggcgaccgagttgctcttgcccggcgtcaatacgggataataccgcgccacatagcagaacttt |
| | gRNA3-CMV- | aaaagtgctcatcattggaaaacgttcttcggggcgaaaactctcaaggatcttaccgctgttgagafccagttcgatgt |
| | dSaCas9-VP64 | aacccactcgtgcacccaactgatcttcagcatcttttactttcaccagcgtttctgggtgagcaaaaacaggaaggca |
| | 5′ part of | aaatgccgcaaaaaagggaataagggcgacacggaaatgttgaatactcatactcttcctttttcaatattattgaagcattt |
| | Ampicillin | atcagggttattgtctcatgagcggatacatatttgaatgtatttagaaaaataaacaaataggggttccgcgcacatttccccga |
| | resistant gene | aaagtgccacctgacgtctaagaaaccattattatcatgacattaacctataaaaataggcgtatcacgaggccctttcgtctcgc |
| | (AmpR) (bold)- | gcgtttcggtgatgacggtgaaaacctctgacacatgcagctcccggagacggtcacagcttgtctgtaagcggatgccggg |
| | AmpR promoter | agcagacaagcccgtcagggcgcgtcagcgggtgttggcgggtgtcggggctggcttaactatgcggcatcagagcagatt |
| | (underlined)-f1 | gtactgagagtgcaccataaaattgtaaacgttaatattttgttaaaattcgcgttaaatttttgttaaatcagctcatttttt |
| | bacteriophage | aaccaatagaccgaaatcggcaaaacccttataaatcaaaagaatagcccgagatagagttgagtgttgttccagtt |
| | origin of | tggaacaagagtccactattaaagaacgtggactccaacgtcaaagggcgaaaaaccgtctatcagggcgatggcc |
| | replication | cactacgtgaaccatcacccaaatcaagttttttggggtcgaggtgccgtaaagcactaaatcggaaccctaaagggagc |
| | (bold)-5′ITR | ccccgatttagagcttgacggggaaagccggcgaacgtggcgagaaaggaagggaagaaagcgaaaggagcgggcgct |
| | (underlined)- | aaggcgctggcaagtgtagcggtcacgctgcgcgtaaccaccacacccgccgcgcttaatgcgccgctacagggcgcgta |
| | U6 promoter | ctatggttgctttgacgtatgcggtgtgaaataccgcacagatgcgtaaggagaaaataccgcatcaggcgcccctgcaggca |
| | (bold)-Shank3 | gctgcgcgctccctcgctcactcaggcccccccgccaaagccccggcgtcgcgccacctttcgtcgcccgccctcagtga |
| | gRNA3 | gcgagcgagcgcgcagagagggagtggccaactccatcactaggggttcctgcggccgcctcgagGAGGGCCT |
| | (underlined)- | ATTTCCCATGATTCCTTCATATTTGCATATACGATACAAGGCTGTTAGA |
| | gRNA scaffold | GAGATAATTGGAATTAATTTGACTGTAAACACAAAGATATTAGTACAAA |
| | (bold)-CMV | ATACGTGACGTAGAAAGTAATAATTTCTTGGGTAGTTTGCAGTTTTAAA |
| | promoter | ATTATGTTTTAAAATGGACTATCATATGCTTACCGTAACTTGAAAGTAT |
| | (underlined)- | TTCGATTTCTTGGCTTTATATATCTTGTGGAAAGGACGAAACACCGCCG |
| | SV40 nuclear | GGAAACTGGGGTCCCCCGGTTATAGTACTCTGGAAACAGAATCTACTATA |
| | localization | ACAAGGCAAAATGCCGTGTTTATCTCGTCAACTTGTTGGCGAGATTTTT |
| | signal (bold)- | TTGCGTTGACATTGATTATTGACTAGTTATTAATAGTAATCAATTACGGGGT |
| | dSaCas9 | CATTAGTTCATAGCCCATATATGGAGTTCCGCGTTACATAACTTACGGTAAA |
| | (underlined)- | TGGCCCGCCTGGCTGACCGCCCAACGACCCCCGCCCATTGACGTCAATAAT |
| | Nucleoplasmin | GACGTATGTTCCCATAGTAACGCCAATAGGGACTTTCCATTGACGTCAATGG |
| | nuclear | GTGGAGTATTTACGGTAAACTGCCCACTTGGCAGTACATCAAGTGTATCATA |
| | localization | TGCCAAGTACGCCCCCTATTGACGTCAATGACGGTAAATGGCCCGCCTGGC |
| | signal (bold)- | ATTATGCCCAGTACATGACCTTATGGGACTTTCCTACTTGGCAGTACATCTA |
| | VP64 | CGTATTAGTCATCGCTATTACCATGGTGATGCGGTTTTGGCAGTACATCAAT |
| | (underlined)- | GGGCGTGGATAGCGGTTTGACTCACGGGGATTTCCAAGTCTCCACCCCATTG |
| | Bovine growth | ACGTCAATGGGAGTTTGTTTTGGCACCAAAATCAACGGGACTTTCCAAAAT |
| | hormone | GTCGTAACAACTCCGCCCCATTGACGCAAATGGGCGGTAGGCGTGTACGGT |
| | polyadenylation | GGGAGGTCTATATAAGCAGAGCTCTCTGGCTAACTACCGGTGCCACCATGG |
| | signal (bold)- | CCCCAAAGAAGAAGCGGAAGGTCGGTATCCACGGAGTCCCAGCAGCCAA |
| | 3′ITR | GCGGAACTACATCCTGGGCCTGGCCATCGGCATCACCAGCGTGGGCTACGG |
| | (underlined)- | CATCATCGACTACGAGACACGGGACGTGATCGATGCCGGCGTGCGGCTGTT |
| | high-copy- | CAAAGAGGCCAACGTGGAAAACAACGAGGGCAGGCGGAGCAAGAGAGGC |
| | number | GCCAGAAGGCTGAAGCGGCGGAGGCGGCATAGAATCCAGAGAGTGAAGAA |
| | ColE1/pMB1/ | GCTGCTGTTCGACTACAACCTGCTGACCGACCACAGCGAGCTGAGCGGCAT |
| | pBR322/pUC | CAACCCCTACGAGGCCAGAGTGAAGGGCCTGAGCCAGAAGCTGAGCGAGG |
| | origin of | AAGAGTTCTCTGCCGCCCTGCTGCACCTGGCCAAGAGAAGAGGCGTGCACA |
| | replication | ACGTGAACGAGGTGGAAGAGGACACCGGCAACGAGCTGTCCACCAAAGAG |
| | (bold)-3′ part | CAGATCAGCCGGAACAGCAAGGCCCTGGAAGAGAAATACGTGGCCGAACT |
| | of Ampicillin | GCAGCTGGAACGGCTGAAGAAAGACGGCGAAGTGCGGGGCAGCATCAACA |
| | resistant gene | GATTCAAGACCAGCGACTACGTGAAAGAAGCCAAACAGCTGCTGAAGGTG |
| | (AmpR) | CAGAAGGCCTACCACCAGCTGGACCAGAGCTTCATCGACACCTACATCGAC |
| | (underlined) | CTGCTGGAAACCCGGCGGACCTACTATGAGGGACCTGGCGAGGGCAGCCCC |
| | | TTCGGCTGGAAGGACATCAAAGAATGGTACGAGATGCTGATGGGCCACTGC |
| | | ACCTACTTCCCCGAGGAACTGCGGAGCGTGAAGTACGCCTACAACGCCGAC |
| | | CTGTACAACGCCCTGAACGACCTGAACAATCTCGTGATCACCAGGGACGAG |
| | | AACGAGAAGCTGGAATATTACGAGAAGTTCCAGATCATCGAGAACGTGTTC |
| | | AAGCAGAAGAAGAAGCCCACCCTGAAGCAGATCGCCAAAGAAATCCTCGT |
| | | GAACGAAGAGGATATTAAGGGCTACAGAGTGACCAGCACCGGCAAGCCCG |
| | | AGTTCACCAACCTGAAGGTGTACCACGACATCAAGGACATTACCGCCCGGA |
| | | AAGAGATTATTGAGAACGCCGAGCTGCTGGATCAGATTGCCAAGATCCTGA |
| | | CCATCTACCAGAGCAGCGAGGACATCCAGGAAGAACTGACCAATCTGAACT |
| | | CCGAGCTGACCCAGGAAGAGATCGAGCAGATCTCTAATCTGAAGGGCTATA |
| | | CCGGCACCCACAACCTGAGCCTGAAGGCCATCAACCTGATCCTGGACGAGC |
| | | TGTGGCACACCAACGACAACCAGATCGCTATCTTCAACCGGCTGAAGCTGG |
| | | TGCCCAAGAAGGTGGACCTGTCCCAGCAGAAAGAGATCCCCACCACCCTGG |
| | | TGGACGACTTCATCCTGAGCCCCGTCGTGAAGAGAAGCTTCATCCAGAGCA |
| | | TCAAAGTGATCAACGCCATCATCAAGAAGTACGGCCTGCCCAACGACATCA |
| | | TTATCGAGCTGGCCCGCGAGAAGAACTCCAAGGACGCCCAGAAAATGATCA |
| | | ACGAGATGCAGAAGCGGAACCGGCAGACCAACGAGCGGATCGAGGAAATC |
| | | ATCCGGACCACCGGCAAAGAGAACGCCAAGTACCTGATCGAGAAGATCAA |
| | | GCTGCACGACATGCAGGAAGGCAAGTGCCTGTACAGCCTGGAAGCCATCCC |
| | | TCTGGAAGATCTGCTGAACAACCCCTTCAACTATGAGGTGGACCACATCAT |
| | | CCCCAGAAGCGTGTCCTTCGACAACAGCTTCAACAACAAGGTGCTCGTGAA |
| | | GCAGGAAGAAGCCAGCAAGAAGGGCAACCGGACCCCATTCCAGTACCTGA |
| | | GCAGCAGCGACAGCAAGATCAGCTACGAAACCTTCAAGAAGCACATCCTGA |
| | | ATCTGGCCAAGGGCAAGGGCAGAATCAGCAAGACCAAGAAAGAGTATCTG |
| | | CTGGAAGAACGGGACATCAACAGGTTCTCCGTGCAGAAAGACTTCATCAAC |
| | | CGGAACCTGGTGGATACCAGATACGCCACCAGAGGCCTGATGAACCTGCTG |
| | | CGGAGCTACTTCAGAGTGAACAACCTGGACGTGAAAGTGAAGTCCATCAAT |
| | | GGCGGCTTCACCAGCTTTCTGCGGCGGAAGTGGAAGTTTAAGAAAGAGCGG |
| | | AACAAGGGGTACAAGCACCACGCCGAGGACGCCCTGATCATTGCCAACGCC |
| | | GATTTCATCTTCAAAGAGTGGAAGAAACTGGACAAGGCCAAAAAAGTGATG |
| | | GAAAACCAGATGTTCGAGGAAAAGCAGGCCGAGAGCATGCCCGAGATCGA |
| | | AACCGAGCAGGAGTACAAAGAGATCTTCATCACCCCCCACCAGATCAAGCA |
| | | CATTAAGGACTTCAAGGACTACAAGTACAGCCACCGGGTGGACAAGAAGCC |
| | | TAATAGAGAGCTGATTAACGACACCCTGTACTCCACCCGGAAGGACGACAA |
| | | GGGCAACACCCTGATCGTGAACAATCTGAACGGCCTGTACGACAAGGACAA |
| | | TGACAAGCTGAAAAAGCTGATCAACAAGAGCCCCGAAAAGCTGCTGATGTA |
| | | CCACCACGACCCCCAGACCTACCAGAAACTGAAGCTGATTATGGAACAGTA |
| | | CGGCGACGAGAAGAATCCCCTGTACAAGTACTACGAGGAAACCGGGAACT |
| | | ACCTGACCAAGTACTCCAAAAAGGACAACGGCCCCGTGATCAAGAAGATTA |
| | | AGTATTACGGCAACAAACTGAACGCCCATCTGGACATCACCGACGACTACC |
| | | CCAACAGCAGAAACAAGGTCGTGAAGCTGTCCCTGAAGCCCTACAGATTCG |
| | | ACGTGTACCTGGACAATGGCGTGTACAAGTTCGTGACCGTGAAGAATCTGG |
| | | ATGTGATCAAAAAAGAAAACTACTACGAAGTGAATAGCAAGTGCTATGAG |
| | | GAAGCTAAGAAGCTGAAGAAGATCAGCAACCAGGCCGAGTTTATCGCCTCC |
| | | TTCTACAACAACGATCTGATCAAGATCAACGGCGAGCTGTATAGAGTCATC |
| | | GGCGTGAACAACGACCTGCTGAACCGGATCGAAGTGAACATGATCGACATC |
| | | ACCTACCGCGAGTACCTGGAAAACATGAACGACAAGAGGCCCCCCAGGATC |
| | | ATTAAGACAATCGCCTCCAAGACCCAGAGCATTAAGAAGTACAGCACAGAC |
| | | ATTCTGGGCAACCTGTATGAAGTGAAATCTAAGAAGCACCCTCAGATCATC |
| | | AAAAAGGGCAAAAGGCCGGCGGCCACGAAAAAGGCCGGCCAGGCAAAA |
| | | AAGAAAAAGGGATCCGAGGCCAGCGGTTCCGGACGGGCTGACGCATTGGA |
| | | CGATTTTGATCTGGATATGCTGGGAAGTGACGCCCTCGATGATTTTGACCTT |
| | | GACATGCTTGGTTCGGATGCCCTTGATGACTTTGACCTCGACATGCTCGGCA |
| | | GTGACGCCCTTGATGATTTCGACCTGGACATGCTGTGATCTAGAGAATTCCT |
| | | AGAGCTCGCTGATCAGCCTCGACTGTGCCTTCTAGTTGCCAGCCATCTGT |
| | | TGTTTGCCCCTCCCCCGTGCCTTCCTTGACCCTGGAAGGTGCCACTCCC |
| | | ACTGTCCTTTCCTAATAAAATGAGGAAATTGCATCGCATTGTCTGAGTA |
| | | GGTGTCATTCTATTCTGGGGGGTGGGGGGGGCAGGACAGCAAGGGG |
| | | GAGGATTGGGAAGAGAATAGCAGGCATGCTGGGGAgctagaggccgcaggaaccc |
| | | ctagtgatggagttcgccactccctctctgcgccctcgctcgctcactgagcccggccgaccaaaggtcgcccgacccccg |
| | | ggcggcctcagtgagcgagcgagcgcgcagctgcctgcaggacatgtgagcaaaaggccagcaaaaggccaggaaccg |
| | | taaaaaggccgcgttgctggcgtttttccataggctccgcccccctgacgagcatcacaaaaatcgacgctcaagtcaga |
| | | ggtggcgaaacccgacaggactataaagataccaggcgtttccccctggaagctccctcgtgcgctctcctgttccgac |
| | | cctgccgcttaccggatacctgtccgcctttctcccttcgggaagcgtggcgctttctcatagctcacgctgtaggtatctc |
| | | agttcggtgtaggtcgttcgctccaagctgggctgtgtgcacgaaccccccgttcagcccgaccgctgcgccttatccggt |
| | | aactatcgtcttgagtccaacccggtaagacacgacttatcgccactggcagcagccactggtaacaggattagcagagcga |
| | | ggtatgtaggcggtgctacagagttcttgaagtggtggcctaactacggctacactagaagaacagtatttggtatctgcgctct |
| | | gctgaagccagttaccttcggaaaaagagttggtagctcttgatccggcaaacaaaccaccgctggtagcggtggtttttttgitt |
| | | gcaagcagcagattacgcgcagaaaaaaaggatctcaagaagatcctttgatcttttctacggggtctgacgctcagtggaac |
| | | gaaaactcacgttaagggattttggtcatgagattatcaaaaaggatcttcacctagatccttttaaattaaaaatgaagttttaaat |
| | | caatctaaagtatatatgagtaaacttggtctgacagttaccaatgcttaatcagtgaggcacctatctcagcgatctctctatttcg |
| | | ttcatccatagttgcctgactccccgtcgtgtagataactacgatacgggagggcttaccatctggccccagtgctgcaatgata |
| | | ccgcggcttccacgctcaccggctccagatttatcagcaataaaccagccagccggaagggccgagcgcagaagtggtcct |
| | | gcaactttatccgcctccatccagtctattaattcttgccgggaagctagagtaagtagttccccagttaatagtttgcgcaacgtt |
| | | gttgccattgctacaggcatcgtgctctcacgctcgtcgtttggtatggcttcattcagctccggcgatcaaggcgagt |
| | | tacatgatcccccatgttctccaaaaaagcgcttagctccttcggtcctccgatccttgtcagaagtaagttggccgcagtcttat |
| | | cactcatggttatgg |
| 12 | AAV-U6 | cagcactgcataattctcttactgtcatgccatccgtaagatgcttttctgtgactggtgagtactcaaccaagtcattctg |
| | Shank3- | agaatagtgtatgcggcgaccgagttgctcttgcccggcgtcaatacgggataataccgcgccacatagcagaacttt |
| | gRNA4-CMV- | aaaagtgctcatcattggaaaacgttcttcggggcgaaaactctcaaggatcttaccgctgttgagatccagttcgatgt |
| | dSaCas9-VP64 | aacccactcgtgcacccaactgatcttcagcatcttttactttcaccagcgtttctgggtgagcaaaaacaggaaggca |
| | 5′ part of | aaatgccgcaaaaaagggaataagggcgacacggaaatgttgaatactcatactcttcctttttcaatattattgaagcattt |
| | Ampicillin | atcaggcttattgtctcatgagcggatacatatttgaatgtatttagaaaaataaacaaataggggttccgcgcacatttccccga |
| | resistant gene | aaagtgccacctgacgtctaagaaaccattattatcatgacattaacctataaaaataggcgtatcacgaggccctttcgtctcgc |
| | (AmpR) (bold)- | gcgtttcggtgatgacggtgaaaacctctgacacatgcagctcccggagacggtcacagcttgtctgtaagcggatgccggg |
| | AmpR promoter | agcagacaagcccgtcaggggcgtcagcgggtgttggcgggtgtcggggctggcttaactatgcggcatcagagcagatt |
| | (underlined)-f1 | gtactgagagtgcaccataaaattgtaaacgttaatattttgttaaaattcgcgttaaatttttgttaaatcagctcatttttt |
| | bacteriophage | aaccaatagaccgaaatcggcaaaatcccttataaatcaaaagaatagcccgagatagagttgagtgttgttccagtt |
| | origin of | tggaacaagagtccactattaaagaacgtggactccaacgtcaaagggcgaaaaaccgtctatcagggcgatggcc |
| | replication | cactacgtgaaccatcacccaaatcaagttttttggggtcgaggtgccgtaaagcactaaatcggaaccctaaagggagc |
| | (bold)-5′ITR | ccccgatttagagcttgacggggaaagccggcgaacgtggcgagaaaggaagggaagaaagcgaaaggagcgggcgct |
| | (underlined)- | aaggcgctggcaagtgtagcggtcacgctgcgcgtaaccaccacacccgccgcgcttaatgcgccgctacagggcgcgta |
| | U6 promoter | ctatggttgctttgacgtatgcggtgtgaaataccgcacagatgcgtaaggagaaaataccgcatcaggcgcccctgcaggca |
| | (bold)-Shank3 | gctgcgcgctcgctcgctcactgagccccccccgccaaagccccggcgtcgggcgacctttcgtcgcccggcctcagtga |
| | gRNA4 | gcgagcgagcgcgcagagagggagtggccaactccatcactaggggttcctgcggccgcctcgagGAGGGCCT |
| | (underlined)- | ATTTCCCATGATTCCTTCATATTTGCATATACGATACAAGGCTGTTAGA |
| | gRNA scaffold | GAGATAATTGGAATTAATTTGACTGTAAACACAAAGATATTAGTACAAA |
| | (bold)-CMV | ATACGTGACGTAGAAAGTAATAATTTCTTGGGTAGTTTGCAGTTTTAAA |
| | promoter | ATTATGTTTTAAAATGGACTATCATATGCTTACCGTAACTTGAAAGTAT |
| | (underlined)- | TTCGATTTCTTGGCTTTATATATCTTGTGGAAAGGACGAAACACCGGGG |
| | SV40 nuclear | GCCCGCGCCGAAGAGGGGGTTATAGTACTCTGGAAACAGAATCTACTAT |
| | localization | AACAAGGCAAAATGCCGTGTTTATCTCGTCAACTTGTTGGCGAGATTTT |
| | signal (bold)- | TTTGCGTTGACATTGATTATTGACTAGTTATTAATAGTAATCAATTACGGGG |
| | dSaCas9 | TCATTAGTTCATAGCCCATATATGGAGTTCCGCGTTACATAACTTACGGTAA |
| | (underlined)- | ATGGCCCGCCTGGCTGACCGCCCAACGACCCCCGCCCATTGACGTCAATAA |
| | Nucleoplasmin | TGACGTATGTTCCCATAGTAACGCCAATAGGGACTTTCCATTGACGTCAATG |
| | nuclear | GGTGGAGTATTTACGGTAAACTGCCCACTTGGCAGTACATCAAGTGTATCAT |
| | localization | ATGCCAAGTACGCCCCCTATTGACGTCAATGACGGTAAATGGCCCGCCTGG |
| | signal (bold)- | CATTATGCCCAGTACATGACCTTATGGGACTTTCCTACTTGGCAGTACATCT |
| | VP64 | ACGTATTAGTCATCGCTATTACCATGGTGATGCGGTTTTGGCAGTACATCAA |
| | (underlined)- | TGGGCGTGGATAGCGGTTTGACTCACGGGGATTTCCAAGTCTCCACCCCATT |
| | Bovine growth | GACGTCAATGGGAGTTTGTTTTGGCACCAAAATCAACGGGACTTTCCAAAA |
| | hormone | TGTCGTAACAACTCCGCCCCATTGACGCAAATGGGGGTAGGCGTGTACGG |
| | polyadenylation | TGGGAGGTCTATATAAGCAGAGCTCTCTGGCTAACTACCGGTGCCACCATG |
| | signal (bold)- | GCCCCAAAGAAGAAGCGGAAGGTCGGTATCCACGGAGTCCCAGCAGCCA |
| | 3′ITR | AGCGGAACTACATCCTGGGCCTGGCCATCGGCATCACCAGCGTGGGCTACG |
| | (underlined)- | GCATCATCGACTACGAGACACGGGACGTGATCGATGCCGGCGTGCGGCTGT |
| | high-copy- | TCAAAGAGGCCAACGTGGAAAACAACGAGGGCAGGCGGAGCAAGAGAGGC |
| | number | GCCAGAAGGCTGAAGCGGCGGAGGCGGCATAGAATCCAGAGAGTGAAGAA |
| | ColE1/pMB1/ | GCTGCTGTTCGACTACAACCTGCTGACCGACCACAGCGAGCTGAGCGGCAT |
| | pBR322/pUC | CAACCCCTACGAGGCCAGAGTGAAGGGCCTGAGCCAGAAGCTGAGCGAGG |
| | origin of | AAGAGTTCTCTGCCGCCCTGCTGCACCTGGCCAAGAGAAGAGGCGTGCACA |
| | replication | ACGTGAACGAGGTGGAAGAGGACACCGGCAACGAGCTGTCCACCAAAGAG |
| | (bold)-3′ part | CAGATCAGCCGGAACAGCAAGGCCCTGGAAGAGAAATACGTGGCCGAACT |
| | of Ampicillin | GCAGCTGGAACGGCTGAAGAAAGACGGCGAAGTGCGGGGCAGCATCAACA |
| | resistant gene | GATTCAAGACCAGCGACTACGTGAAAGAAGCCAAACAGCTGCTGAAGGTG |
| | (AmpR) | CAGAAGGCCTACCACCAGCTGGACCAGAGCTTCATCGACACCTACATCGAC |
| | (underlined) | CTGCTGGAAACCCGGCGGACCTACTATGAGGGACCTGGCGAGGGCAGCCCC |
| | | TTCGGCTGGAAGGACATCAAAGAATGGTACGAGATGCTGATGGGCCACTGC |
| | | ACCTACTTCCCCGAGGAACTGCGGAGCGTGAAGTACGCCTACAACGCCGAC |
| | | CTGTACAACGCCCTGAACGACCTGAACAATCTCGTGATCACCAGGGACGAG |
| | | AACGAGAAGCTGGAATATTACGAGAAGTTCCAGATCATCGAGAACGTGTTC |
| | | AAGCAGAAGAAGAAGCCCACCCTGAAGCAGATCGCCAAAGAAATCCTCGT |
| | | GAACGAAGAGGATATTAAGGGCTACAGAGTGACCAGCACCGGCAAGCCCG |
| | | AGTTCACCAACCTGAAGGTGTACCACGACATCAAGGACATTACCGCCCGGA |
| | | AAGAGATTATTGAGAACGCCGAGCTGCTGGATCAGATTGCCAAGATCCTGA |
| | | CCATCTACCAGAGCAGCGAGGACATCCAGGAAGAACTGACCAATCTGAACT |
| | | CCGAGCTGACCCAGGAAGAGATCGAGCAGATCTCTAATCTGAAGGGCTATA |
| | | CCGGCACCCACAACCTGAGCCTGAAGGCCATCAACCTGATCCTGGACGAGC |
| | | TGTGGCACACCAACGACAACCAGATCGCTATCTTCAACCGGCTGAAGCTGG |
| | | TGCCCAAGAAGGTGGACCTGTCCCAGCAGAAAGAGATCCCCACCACCCTGG |
| | | TGGACGACTTCATCCTGAGCCCCGTCGTGAAGAGAAGCTTCATCCAGAGCA |
| | | TCAAAGTGATCAACGCCATCATCAAGAAGTACGGCCTGCCCAACGACATCA |
| | | TTATCGAGCTGGCCCGCGAGAAGAACTCCAAGGACGCCCAGAAAATGATCA |
| | | ACGAGATGCAGAAGCGGAACCGGCAGACCAACGAGCGGATCGAGGAAATC |
| | | ATCCGGACCACCGGCAAAGAGAACGCCAAGTACCTGATCGAGAAGATCAA |
| | | GCTGCACGACATGCAGGAAGGCAAGTGCCTGTACAGCCTGGAAGCCATCCC |
| | | TCTGGAAGATCTGCTGAACAACCCCTTCAACTATGAGGTGGACCACATCAT |
| | | CCCCAGAAGCGTGTCCTTCGACAACAGCTTCAACAACAAGGIGCTCGTGAA |
| | | GCAGGAAGAAGCCAGCAAGAAGGGCAACCGGACCCCATTCCAGTACCTGA |
| | | GCAGCAGCGACAGCAAGATCAGCTACGAAACCTTCAAGAAGCACATCCTGA |
| | | ATCTGGCCAAGGGCAAGGGCAGAATCAGCAAGACCAAGAAAGAGTATCTG |
| | | CTGGAAGAACGGGACATCAACAGGTTCTCCGTGCAGAAAGACTTCATCAAC |
| | | CGGAACCTGGTGGATACCAGATACGCCACCAGAGGCCTGATGAACCTGCTG |
| | | CGGAGCTACTTCAGAGTGAACAACCTGGACGTGAAAGTGAAGTCCATCAAT |
| | | GGCGGCTTCACCAGCTTTCTGCGGCGGAAGTGGAAGTTTAAGAAAGAGCGG |
| | | AACAAGGGGTACAAGCACCACGCCGAGGACGCCCTGATCATTGCCAACGCC |
| | | GATTTCATCTTCAAAGAGTGGAAGAAACTGGACAAGGCCAAAAAAGTGATG |
| | | GAAAACCAGATGTTCGAGGAAAAGCAGGCCGAGAGCATGCCCGAGATCGA |
| | | AACCGAGCAGGAGTACAAAGAGATCTTCATCACCCCCCACCAGATCAAGCA |
| | | CATTAAGGACTTCAAGGACTACAAGTACAGCCACCGGGTGGACAAGAAGCC |
| | | TAATAGAGAGCTGATTAACGACACCCTGTACTCCACCCGGAAGGACGACAA |
| | | GGGCAACACCCTGATCGTGAACAATCTGAACGGCCTGTACGACAAGGACAA |
| | | TGACAAGCTGAAAAAGCTGATCAACAAGAGCCCCGAAAAGCTGCTGATGTA |
| | | CCACCACGACCCCCAGACCTACCAGAAACTGAAGCTGATTATGGAACAGTA |
| | | CGGCGACGAGAAGAATCCCCTGTACAAGTACTACGAGGAAACCGGGAACT |
| | | ACCTGACCAAGTACTCCAAAAAGGACAACGGCCCCGTGATCAAGAAGATTA |
| | | AGTATTACGGCAACAAACTGAACGCCCATCIGGACATCACCGACGACTACC |
| | | CCAACAGCAGAAACAAGGTCGTGAAGCTGTCCCTGAAGCCCTACAGATTCG |
| | | ACGTGTACCTGGACAATGGCGTGTACAAGTTCGTGACCGTGAAGAATCTGG |
| | | ATGTGATCAAAAAAGAAAACTACTACGAAGTGAATAGCAAGTGCTATGAG |
| | | GAAGCTAAGAAGCTGAAGAAGATCAGCAACCAGGCCGAGTTTATCGCCTCC |
| | | TTCTACAACAACGATCTGATCAAGATCAACGGCGAGCTGTATAGAGTGATC |
| | | GGCGTGAACAACGACCTGCTGAACCGGATCGAAGTGAACATGATCGACATC |
| | | ACCTACCGCGAGTACCTGGAAAACATGAACGACAAGAGGCCCCCCAGGATC |
| | | ATTAAGACAATCGCCTCCAAGACCCAGAGCATTAAGAAGTACAGCACAGAC |
| | | ATTCTGGGCAACCTGTATGAAGTGAAATCTAAGAAGCACCCTCAGATCATC |
| | | AAAAAGGGCAAAAGGCCGGCGGCCACGAAAAAGGCCGGCCAGGCAAAA |
| | | AAGAAAAAGGGATCCGAGGCCAGCGGTTCCGGACGGGCTGACGCATTGGA |
| | | CGATTTTGATCTGGATATGCTGGGAAGTGACGCCCTCGATGATTTTGACCTT |
| | | GACATGCTTGGTTCGGATGCCCTTGATGACTTTGACCTCGACATGCTCGGCA |
| | | GTGACGCCCTTGATGATTTCGACCTGGACATGCTGTGATCTAGAGAATTCCT |
| | | AGAGCTCGCTGATCAGCCTCGACTGTGCCTTCTAGTTGCCAGCCATCTGT |
| | | TGTTTGCCCCTCCCCCGTGCCTTCCTTGACCCTGGAAGGTGCCACTCCC |
| | | ACTGTCCTTTCCTAATAAAATGAGGAAATTGCATCGCATTGTCTGAGTA |
| | | GGTGTCATTCTATTCTGGGGGGTGGGGGGGGCAGGACAGCAAGGGG |
| | | GAGGATTGGGAAGAGAATAGCAGGCATGCTGGGGAgctagaggccgcaggaaccc |
| | | ctagtgatggagttggccactccctctctgcgcgctcgctcgctcactgaggccgggcgaccaaaggtcgcccgacgcccg |
| | | ggcggcctcagtgagcgagcgagcgcgcagctgcctgcaggacatgtgagcaaaaggccagcaaaaggccaggaaccg |
| | | taaaaaggccgcgttgctggcgtttttccataggctccgcccccctgacgagcatcacaaaaatcgacgctcaagtcaga |
| | | ggtggcgaaacccgacaggactataaagataccaggcgtttccccctggaagctccctcgtgcgctctcctgttccgac |
| | | cctgccgcttaccggatacctgtccgcctttctcccttcgggaagcgtggcgctttctcatagctcacgctgtaggtatctc |
| | | agttcggtgtaggtcgttcgctccaagctgggctgtgtgcacgaaccccccgttcagcccgaccgctgcgccttatccggt |
| | | aactatcgtcttgagtccaacccggtaagacacgacttatcgccactggcagcagccactggtaacaggattagcagagcga |
| | | ggtatgtaggggtgctacagagttcttgaagtggtggcctaactacggctacactagaagaacagtatttggtatctgcgctct |
| | | gctgaagccagttaccttcggaaaaagagttggtagctcttgatccggcaaacaaaccaccgctggtagcggtggtttttttgttt |
| | | gcaagcagcagattacgcgcagaaaaaaaggatctcaagaagatcctttgatcttttctacggggtctgacgctcagtggaac |
| | | gaaaactcacgttaagggattttggtcatgagattatcaaaaaggatcttcacctagatccttttaaattaaaaatgaagttttaaat |
| | | caatctaaagtatatatgagtaaacttggtctgacagttaccaatgcttaatcagtgaggcacctatctcagcgatctgtctatttcg |
| | | ttcatccatagttgcctgactccccgtcgtgtagataactacgatacgggagggcttaccatctggccccagtgctgcaatgata |
| | | ccgcggcttccacgctcaccggctccagatttatcagcaataaaccagccagccggaagggccgagcgcagaagtggtcct |
| | | gcaactttatccgcctccatccagtctattaattcttgcccggaagctagagtaagtagttccccagttaatagtttgcgcaacgtt |
| | | gttgccattgctacaggcatcgtggtgtcacgctcgtcgtttggtatggcttcattcagctccggttcccaacgatcaaggcgagt |
| | | tacatgatcccccatgttctgcaaaaaagcgcttagctccttcggtcctccgatccttgtcagaagtaagttggccgcagtgttat |
| | | cactcatggttatgg |
| 13 | AAV-U6- | cagcactgcataattctcttactgtcatgccatccgtaagatgcttttctgtgactggtgagtactcaaccaagtcattctg |
| | Shank3- | agaatagtgtatgcggcgaccgagttgctcttgcccggcgtcaatacgggataataccgcgccacatagcagaacttt |
| | gRNA5-CMV- | aaaagtgctcatcattggaaaacgttcttcggggcgaaaactctcaaggatcttaccgctgttgagatccagttcgatgt |
| | dSaCas9-VP64 | aacccactcgtgcacccaactgatcttcagcatcttttactttcaccagcgtttctgggtgagcaaaaacaggaaggca |
| | 5′ part of | aaatgccgcaaaaaagggaataaggggacacggaaatgttgaatactcatactcttcctttttcaatattattgaagcattt |
| | Ampicillin | atcaggcttattgtctcatgagcggatacatatttgaatgtatttagaaaaataaacaaataggggttccgcgcacatttccccga |
| | resistant gene | aaagtgccacctgacgtctaagaaaccattattatcatgacattaacctataaaaataggcgtatcacgaggccctttcgtctcgc |
| | (AmpR) (bold)- | gcgtttcggtgatgacggtgaaaacctctgacacatgcagctcccggagacggtcacagcttgtctgtaagcggatgccggg |
| | AmpR promoter | agcagacaagcccgtcagggcgcgtcaggggtgttggcgggtgtcggggctggcttaactatgcggcatcagagcagatt |
| | (underlined)-f1 | gtactgagagtgcaccataaaattgtaaacgttaatattttgttaaaattcgcgttaaatttttgttaaatcagctcatttttt |
| | bacteriophage | aaccaatagaccgaaatcggcaaaatcccttataaatcaaaagaatagcccgagatagagttgagtgttgttccagtt |
| | origin of | tggaacaagagtccactattaaagaacgtggactccaacgtcaaagggcgaaaaaccgtctatcagggcgatggcc |
| | replication | cactacgtgaaccatcacccaaatcaagttttttggggtcgaggtgccgtaaagcactaaatcggaaccctaaagggagc |
| | (bold)-5′ITR | ccccgatttagagcttgacggggaaagccggcgaacgtggcgagaaaggaagggaagaaagcgaaaggaggggcgct |
| | (underlined)- | aaggcgctggcaagtgtagcggtcacgctgcgcgtaaccaccacacccgccgcgcttaatgcgcgctacagggcgcgta |
| | U6 promoter | ctatggttgctttgacgtatgcggtgtgaaataccgcacagatgcgtaaggagaaaataccgcatcaggcgcccctgcaggca |
| | (bold)-Shank3 | gctgcgccctccctcgctcactcaggccccccgggcaaagcccgggcgtcggccgacctttcgtcgcccggcctcagtga |
| | gRNA5 | gcgagcgagcgcgcagagagggagtggccaactccatcactaggggttcctgcggccgcctcgagGAGGGCCT |
| | (underlined)- | ATTTCCCATGATTCCTTCATATTTGCATATACGATACAAGGCTGTTAGA |
| | gRNA scaffold | GAGATAATTGGAATTAATTTGACTGTAAACACAAAGATATTAGTACAAA |
| | (bold)-CMV | ATACGTGACGTAGAAAGTAATAATTTCTTGGGTAGTTTGCAGTTTTAAA |
| | promoter | ATTATGTTTTAAAATGGACTATCATATGCTTACCGTAACTTGAAAGTAT |
| | (underlined)- | TTCGATTTCTTGGCTTTATATATCTTGTGGAAAGGACGAAACACCGGGG |
| | SV40 nuclear | GAGTGGCTGACACAGCGGGTTATAGTACTCTGGAAACAGAATCTACTAT |
| | localization | AACAAGGCAAAATGCCGTGTTTATCTCGTCAACTTGTTGGCGAGATTTT |
| | signal (bold)- | TTTGCGTTGACATTGATTATTGACTAGTTATTAATAGTAATCAATTACGGGG |
| | dSaCas9 | TCATTAGTTCATAGCCCATATATGGAGTTCCGCGTTACATAACTTACGGTAA |
| | (underlined)- | ATGGCCCGCCTGGCTGACCGCCCAACGACCCCCGCCCATTGACGTCAATAA |
| | Nucleoplasmin | TGACGTATGTTCCCATAGTAACGCCAATAGGGACTTTCCATTGACGTCAATG |
| | nuclear | GGTGGAGTATTTACGGTAAACTGCCCACTTGGCAGTACATCAAGTGTATCAT |
| | localization | ATGCCAAGTACGCCCCCTATTGACGTCAATGACGGTAAATGGCCCGCCTGG |
| | signal (bold)- | CATTATGCCCAGTACATGACCTTATGGGACTTTCCTACTTGGCAGTACATCT |
| | VP64 | ACGTATTAGTCATCGCTATTACCATGGTGATGCGGTTTTGGCAGTACATCAA |
| | (underlined)- | TGGGCGTGGATAGCGGTTTGACTCACGGGGATTTCCAAGTCTCCACCCCATT |
| | Bovine growth | GACGTCAATGGGAGTTTGTTTTGGCACCAAAATCAACGGGACTTTCCAAAA |
| | hormone | TGTCGTAACAACTCCGCCCCATTGACGCAAATGGGCGGTAGGCGTGTACGG |
| | polyadenylation | TGGGAGGTCTATATAAGCAGAGCTCTCTGGCTAACTACCGGTGCCACCATG |
| | signal (bold)- | GCCCCAAAGAAGAAGCGGAAGGTCGGTATCCACGGAGTCCCAGCAGCCA |
| | 3′ITR | AGCGGAACTACATCCTGGGCCTGGCCATCGGCATCACCAGCGTGGGCTACG |
| | (underlined)- | GCATCATCGACTACGAGACACGGGACGTGATCGATGCCGGCGTGCGGCTGT |
| | high-copy- | TCAAAGAGGCCAACGTGGAAAACAACGAGGGCAGGCGGAGCAAGAGAGGC |
| | number | GCCAGAAGGCTGAAGCGGCGGAGGCGGCATAGAATCCAGAGAGTGAAGAA |
| | ColE1/pMB1/ | GCTGCTGTTCGACTACAACCTGCTGACCGACCACAGCGAGCTGAGCGGCAT |
| | pBR322/pUC | CAACCCCTACGAGGCCAGAGTGAAGGGCCTGAGCCAGAAGCTGAGCGAGG |
| | origin of | AAGAGTTCTCTGCCGCCCTGCTGCACCTGGCCAAGAGAAGAGGCGTGCACA |
| | replication | ACGTGAACGAGGTGGAAGAGGACACCGGCAACGAGCTGTCCACCAAAGAG |
| | (bold)-3′ part | CAGATCAGCCGGAACAGCAAGGCCCTGGAAGAGAAATACGTGGCCGAACT |
| | of Ampicillin | GCAGCTGGAACGGCTGAAGAAAGACGGCGAAGTGCGGGGCAGCATCAACA |
| | resistant gene | GATTCAAGACCAGCGACTACGTGAAAGAAGCCAAACAGCTGCTGAAGGTG |
| | (AmpR) | CAGAAGGCCTACCACCAGCTGGACCAGAGCTTCATCGACACCTACATCGAC |
| | (underlined) | CTGCTGGAAACCCGGCGGACCTACTATGAGGGACCTGGCGAGGGCAGCCCC |
| | | TTCGGCTGGAAGGACATCAAAGAATGGTACGAGATGCTGATGGGCCACTGC |
| | | ACCTACTTCCCCGAGGAACTGCGGAGCGTGAAGTACGCCTACAACGCCGAC |
| | | CTGTACAACGCCCTGAACGACCTGAACAATCTCGTGATCACCAGGGACGAG |
| | | AACGAGAAGCTGGAATATTACGAGAAGTTCCAGATCATCGAGAACGTGTTC |
| | | AAGCAGAAGAAGAAGCCCACCCTGAAGCAGATCGCCAAAGAAATCCTCGT |
| | | GAACGAAGAGGATATTAAGGGCTACAGAGTGACCAGCACCGGCAAGCCCG |
| | | AGTTCACCAACCTGAAGGTGTACCACGACATCAAGGACATTACCGCCCGGA |
| | | AAGAGATTATTGAGAACGCCGAGCTGCTGGATCAGATTGCCAAGATCCTGA |
| | | CCATCTACCAGAGCAGCGAGGACATCCAGGAAGAACTGACCAATCTGAACT |
| | | CCGAGCTGACCCAGGAAGAGATCGAGCAGATCTCTAATCTGAAGGGCTATA |
| | | CCGGCACCCACAACCTGAGCCTGAAGGCCATCAACCTGATCCTGGACGAGC |
| | | TGTGGCACACCAACGACAACCAGATCGCTATCTTCAACCGGCTGAAGCTGG |
| | | TGCCCAAGAAGGTGGACCTGTCCCAGCAGAAAGAGATCCCCACCACCCTGG |
| | | TGGACGACTTCATCCTGAGCCCCGTCGTGAAGAGAAGCTTCATCCAGAGCA |
| | | TTCAAAGTGATCAACGCCATCATCAAGAAGTACGGCCTGCCCAACGACATCA |
| | | TTATCGAGCTGGCCCGCGAGAAGAACTCCAAGGACGCCCAGAAAATGATCA |
| | | ACGAGATGCAGAAGCGGAACCGGCAGACCAACGAGCGGATCGAGGAAATC |
| | | ATCCGGACCACCGGCAAAGAGAACGCCAAGTACCTGATCGAGAAGATCAA |
| | | GCTGCACGACATGCAGGAAGGCAAGTGCCTGTACAGCCTGGAAGCCATCCC |
| | | TCTGGAAGATCTGCTGAACAACCCCTTCAACTATGAGGTGGACCACATCAT |
| | | CCCCAGAAGCGTGTCCTTCGACAACAGCTTCAACAACAAGGIGCTCGTGAA |
| | | CCAGGAAGAAGCCAGCAAGAAGGGCAACCGGACCCCATTCCAGTACCTGA |
| | | GCAGCAGCGACAGCAAGATCAGCTACGAAACCTTCAAGAAGCACATCCTGA |
| | | ATCTGGCCAAGGGCAAGGGCAGAATCAGCAAGACCAAGAAAGAGTATCTG |
| | | CTGGAAGAACGGGACATCAACAGGTTCTCCGTGCAGAAAGACTTCATCAAC |
| | | CGGAACCTGGTGGATACCAGATACGCCACCAGAGGCCTGATGAACCTGCTG |
| | | CGGAGCTACTTCAGAGTGAACAACCTGGACGTGAAAGTGAAGTCCATCAAT |
| | | GGCGGCTTCACCAGCTTTCTGCGGCGGAAGTGGAAGTTTAAGAAAGAGCGG |
| | | AACAAGGGGTACAAGCACCACGCCGAGGACGCCCTGATCATTGCCAACGCC |
| | | GATTTCATCTTCAAAGAGTGGAAGAAACTGGACAAGGCCAAAAAAGTGATG |
| | | GAAAACCAGATGTTCGAGGAAAAGCAGGCCGAGAGCATGCCCGAGATCGA |
| | | AACCGAGCAGGAGTACAAAGAGATCTTCATCACCCCCCACCAGATCAAGCA |
| | | CATTAAGGACTTCAAGGACTACAAGTACAGCCACCGGGTGGACAAGAAGCC |
| | | TAATAGAGAGCTGATTAACGACACCCTGTACTCCACCCGGAAGGACGACAA |
| | | GGGCAACACCCTGATCGTGAACAATCTGAACGGCCTGTACGACAAGGACAA |
| | | TGACAAGCTGAAAAAGCTGATCAACAAGAGCCCCGAAAAGCTGCTGATGTA |
| | | CCACCACGACCCCCAGACCTACCAGAAACTGAAGCTGATTATGGAACAGTA |
| | | CGGCGACGAGAAGAATCCCCTGTACAAGTACTACGAGGAAACCGGGAACT |
| | | ACCTGACCAAGTACTCCAAAAAGGACAACGGCCCCGTGATCAAGAAGATTA |
| | | AGTATTACGGCAACAAACTGAACGCCCATCTGGACATCACCGACGACTACC |
| | | CCAACAGCAGAAACAAGGTCGTGAAGCTGTCCCTGAAGCCCTACAGATTCG |
| | | ACGTGTACCTGGACAATGGCGTGTACAAGTTCGTGACCGTGAAGAATCTGG |
| | | ATGTGATCAAAAAAGAAAACTACTACGAAGTGAATAGCAAGIGCTATGAG |
| | | GAAGCTAAGAAGCTGAAGAAGATCAGCAACCAGGCCGAGTTTATCGCCTCC |
| | | TTCTACAACAACGATCTGATCAAGATCAACGGCGAGCTGTATAGAGTGATC |
| | | GGCGTGAACAACGACCTGCTGAACCGGATCGAAGTGAACATGATCGACATC |
| | | ACCTACCGCGAGTACCTGGAAAACATGAACGACAAGAGGCCCCCCAGGATC |
| | | ATTAAGACAATCGCCTCCAAGACCCAGAGCATTAAGAAGTACAGCACAGAC |
| | | ATTCTGGGCAACCTGTATGAAGTGAAATCTAAGAAGCACCCTCAGATCATC |
| | | AAAAAGGGCAAAAGGCCGGCGGCCACGAAAAAGGCCGGCCAGGCAAAA |
| | | AAGAAAAAGGGATCCGAGGCCAGCGGTTCCGGACGGGCTGACGCATTGGA |
| | | CGATTTTGATCTGGATATGCTGGGAAGTGACGCCCTCGATGATTTTGACCTT |
| | | GACATGCTTGGTTCGGATGCCCTTGATGACTTTGACCTCGACATGCTCGGCA |
| | | GTGACGCCCTTGATGATTTCGACCTGGACATGCTGTGATCTAGAGAATTCCT |
| | | AGAGCTCGCTGATCAGCCTCGACTGTGCCTTCTAGTTGCCAGCCATCTGT |
| | | TGTTTGCCCCTCCCCCGTGCCTTCCTTGACCCTGGAAGGTGCCACTCCC |
| | | ACTGTCCTTTCCTAATAAAATGAGGAAATTGCATCGCATTGTCTGAGTA |
| | | GGTGTCATTCTATTCTGGGGGGTGGGGTGGGGCAGGACAGCAAGGGG |
| | | GAGGATTGGGAAGAGAATAGCAGGCATGCTGGGGAgctagaggccgcaggaaccc |
| | | ctagtgatggagttggccactccctctctgcgcgctcgctcgctcactgagcccgggcgaccaaaggtcgcccgacgcccg |
| | | ggcggcctcagtgagcgagcgagcgcgcagctgcctgcaggacatgtgagcaaaaggccagcaaaaggccaggaaccg |
| | | taaaaaggccgcgttgctggcgtttttccataggctccgcccccctgacgagcatcacaaaaatcgacgctcaagtcaga |
| | | ggtggcgaaacccgacaggactataaagataccaggcgtttccccctggaagctccctcgtgcgctctcctgttccgac |
| | | cctgccgcttaccggatacctgtccgcctttctcccttcgggaagcgtggcgctttctcatagctcacgctgtaggtatctc |
| | | agttcggtgtaggtcgttcgctccaagctgggctgtgtgcacgaaccccccgttcagcccgaccgctgcgccttatccggt |
| | | aactatcgtcttgagtccaacccggtaagacacgacttatcgccactggcagcagccactggtaacaggattagcagagcga |
| | | ggtatgtaggcggtgctacagagttcttgaagtggtggcctaactacggctacactagaagaacagtatttggtatctgcgctct |
| | | gctgaagccagttaccttcggaaaaagagttggtagctcttgatccggcaaacaaaccaccgctggtagcggtggtttttttgttt |
| | | gcaagcagcagattacgcgcagaaaaaaaggatctcaagaagatcctttgatcttttctacggggtctgacgctcagtggaac |
| | | gaaaactcacgttaagggattttggtcatgagattatcaaaaaggatcttcacctagatccttttaaattaaaaatgaagttttaaat |
| | | caatctaaagtatatatgagtaaacttggtctgacagttaccaatgcttaatcagtgaggcacctatctcagcgatctgtctatttcg |
| | | ttcatccatagttgcctgactccccgtcgtgtagataactacgatacgggagggcttaccatctggccccagtgctgcaatgata |
| | | ccgcggcttccacgctcaccggctccagatttatcagcaataaaccagccagccggaagggccgagcgcagaagtggtcct |
| | | gcaactttatccgcctccatccagtctattaattgttgccgggaagctagagtaagtagttcgccagttaatagtttgcgcaacgtt |
| | | gttgccattgctacaggcatcgtggtgtcacgctcgtcgtttggtatggcttcattcagctccggttcccaacgatcaaggcgagt |
| | | tacatgatcccccatgttgtgcaaaaagcggttagctccttcggtcctccgatcgttgtcagaagtaagttggccgcagtgttat |
| | | cactcatggttatgg |
| 14 | AAV-U6- | cagcactgcataattctcttactgtcatgccatccgtaagatgcttttctgtgactggtgagtactcaaccaagtcattctg |
| | Shank3- | agaatagtgtatgcggcgaccgagttgctcttgcccggcgtcaatacgggataataccgcgccacatagcagaacttt |
| | gRNA6-CMV- | aaaagtgctcatcattggaaaacgttcttcggggcgaaaactctcaaggatcttaccgctgttgagatccagttcgatgt |
| | dSaCas9-Vp64 | aacccactcgtgcacccaactgatcttcagcatcttttactttcaccagcgtttctgggtgagcaaaaacaggaaggca |
| | 5′ part of | aaatgccgcaaaaaagggaataagggcgacacggaaatgttgaatactcatactcttcctttttcaatattattgaagcattt |
| | Ampicillin | atcagggttattctctcatgagcggatacatatttgaatctatttagaaaaataaacaaataggggttccgcgcacatttccccga |
| | resistant gene | aaagtgccacctgacgtctaagaaaccattattatcatgacattaacctataaaaataggcgtatcacgaggccctttcgtctcgc |
| | (AmpR) (bold)- | gcgtttcggtgatgacggtgaaaacctctgacacatgcagctcccggagacggtcacagcttgtctgtaagcggatgccggg |
| | AmpR promoter | agcagacaagcccgtcagggcgcgtcagcgggtgttggcgggtgtcggggctggcttaactatgcggcatcagagcagatt |
| | (underlined)-f1 | gtactgagagtgcaccataaaattgtaaacgttaatattttgttaaaattcgcgttaaatttttgttaaatcagctcatttttt |
| | bacteriophage | aaccaatagaccgaaatcggcaaaatcccttataaatcaaaagaatagcccgagatagagttgagtgttgttccagtt |
| | origin of | tggaacaagagtccactattaaagaacgtggactccaacgtcaaagggcgaaaaaccgtctatcagggcgatggcc |
| | replication | cactacgtgaaccatcacccaaatcaagttttttggggtcgaggtgccgtaaagcactaaatcggaaccctaaagggagc |
| | (bold)-5′ITR | ccccgatttagagcttgacggggaaagccggcgaacgtggcgagaaaggaagggaagaaagcgaaaggagcgggcgct |
| | (underlined)- | aaggcgctggcaagtgtagcggtcacgctgcgcgtaaccaccacacccgccgcgcttaatgcgccgctacagggcgcgta |
| | U6 promoter | ctatggttgctttgacgtatgcggtgtgaaataccgcacagatgcgtaaggagaaaataccgcatcaggcgcccctgcaggca |
| | (bold)-Shank3 | gctgcgcgctcgctcgctcactgaggccgcccgggcaaagcccgggcgtcgggcgacctttggtcgcccggcctcagtga |
| | gRNA6 | gcgagcgagcgcgcagagagggagtggccaactccatcactaggggttcctgcggccgcctcgagGAGGGCCT |
| | (underlined)- | ATTTCCCATGATTCCTTCATATTTGCATATACGATACAAGGCTGTTAGA |
| | gRNA scaffold | GAGATAATTGGAATTAATTTGACTGTAAACACAAAGATATTAGTACAAA |
| | (bold)-CMV | ATACGTGACGTAGAAAGTAATAATTTCTTGGGTAGTTTGCAGTTTTAAA |
| | promoter | ATTATGTTTTAAAATGGACTATCATATGCTTACCGTAACTTGAAAGTAT |
| | (underlined)- | TTCGATTTCTTGGCTTTATATATCTTGTGGAAAGGACGAAACACCGGGG |
| | SV40 nuclear | TGACGGAGGGTTCTAGAGGTTATAGTACTCTGGAAACAGAATCTACTATA |
| | localization | ACAAGGCAAAATGCCGTGTTTATCTCGTCAACTTGTTGGCGAGATTTTT |
| | signal (bold)- | TTGCGTTGACATTGATTATTGACTAGTTATTAATAGTAATCAATTACGGGGT |
| | dSaCas9 | CATTAGTTCATAGCCCATATATGGAGTTCCGCGTTACATAACTTACGGTAAA |
| | (underlined)- | TGGCCCGCCTGGCTGACCGCCCAACGACCCCCGCCCATTGACGTCAATAAT |
| | Nucleoplasmin | GACGTATGTTCCCATAGTAACGCCAATAGGGACTTTCCATTGACGTCAATGG |
| | nuclear | GTGGAGTATTTACGGTAAACTGCCCACTTGGCAGTACATCAAGTGTATCATA |
| | localization | TGCCAAGTACGCCCCCTATTGACGTCAATGACGGTAAATGGCCCGCCTGGC |
| | signal (bold)- | ATTATGCCCAGTACATGACCTTATGGGACTTTCCTACTTGGCAGTACATCTA |
| | VP64 | CGTATTAGTCATCGCTATTACCATGGTGATGCGGTTTTGGCAGTACATCAAT |
| | (underlined)- | GGGCGTGGATAGCGGTTTGACTCACGGGGATTTCCAAGTCTCCACCCCATTG |
| | Bovine growth | ACGTCAATGGGAGTTTGTTTTGGCACCAAAATCAACGGGACTTTCCAAAAT |
| | hormone | GTCGTAACAACTCCGCCCCATTGACGCAAATGGGCGGTAGGCGTGTACGGT |
| | polyadenylation | GGGAGGTCTATATAAGCAGAGCTCTCTGGCTAACTACCGGTGCCACCATGG |
| | signal (bold)- | CCCCAAAGAAGAAGCGGAAGGTCGGTATCCACGGAGTCCCAGCAGCCAA |
| | 3′ITR | GCGGAACTACATCCTGGGCCTGGCCATCGGCATCACCAGCGTGGGCTACGG |
| | (underlined)- | CATCATCGACTACGAGACACGGGACGTGATCGATGCCGGCGTGCGGCTGTT |
| | high-copy- | CAAAGAGGCCAACGTGGAAAACAACGAGGGCAGGCGGAGCAAGAGAGGC |
| | number | GCCAGAAGGCTGAAGCGGCGGAGGCGGCATAGAATCCAGAGAGTGAAGAA |
| | ColE1/pMB1/ | GCTGCTGTTCGACTACAACCTGCTGACCGACCACAGCGAGCTGAGCGGCAT |
| | pBR322/pUC | CAACCCCTACGAGGCCAGAGTGAAGGGCCTGAGCCAGAAGCTGAGCGAGG |
| | origin of | AAGAGTTCTCTGCCGCCCTGCTGCACCTGGCCAAGAGAAGAGGCGTGCACA |
| | replication | ACGTGAACGAGGTGGAAGAGGACACCGGCAACGAGCTGTCCACCAAAGAG |
| | (bold)-3′ part | CAGATCAGCCGGAACAGCAAGGCCCTGGAAGAGAAATACGTGGCCGAACT |
| | of Ampicillin | GCAGCTGGAACGGCTGAAGAAAGACGGCGAAGTGCGGGGCAGCATCAACA |
| | resistant gene | GATTCAAGACCAGCGACTACGTGAAAGAAGCCAAACAGCTGCTGAAGGTG |
| | (AmpR) | CAGAAGGCCTACCACCAGCTGGACCAGAGCTTCATCGACACCTACATCGAC |
| | (underlined) | CTGCTGGAAACCCGGCGGACCTACTATGAGGGACCTGGCGAGGGCAGCCCC |
| | | TTCGGCTGGAAGGACATCAAAGAATGGTACGAGATGCTGATGGGCCACTGC |
| | | ACCTACTTCCCCGAGGAACTGCGGAGCGTGAAGTACGCCTACAACGCCGAC |
| | | CTGTACAACGCCCTGAACGACCTGAACAATCTCGTGATCACCAGGGACGAG |
| | | AACGAGAAGCTGGAATATTACGAGAAGTTCCAGATCATCGAGAACGTGTTC |
| | | AAGCAGAAGAAGAAGCCCACCCTGAAGCAGATCGCCAAAGAAATCCTCGT |
| | | GAACGAAGAGGATATTAAGGGCTACAGAGTGACCAGCACCGGCAAGCCCG |
| | | AGTTCACCAACCTGAAGGTGTACCACGACATCAAGGACATTACCGCCCGGA |
| | | AAGAGATTATTGAGAACGCCGAGCTGCTGGATCAGATTGCCAAGATCCTGA |
| | | CCATCTACCAGAGCAGCGAGGACATCCAGGAAGAACTGACCAATCTGAACT |
| | | CCGAGCTGACCCAGGAAGAGATCGAGCAGATCTCTAATCTGAAGGGCTATA |
| | | CCGGCACCCACAACCTGAGCCTGAAGGCCATCAACCTGATCCTGGACGAGC |
| | | TGTGGCACACCAACGACAACCAGATCGCTATCTTCAACCGGCTGAAGCTGG |
| | | TGCCCAAGAAGGTGGACCTGTCCCAGCAGAAAGAGATCCCCACCACCCTGG |
| | | TGGACGACTTCATCCTGAGCCCCGTCGTGAAGAGAAGCTTCATCCAGAGCA |
| | | TCAAAGTGATCAACGCCATCATCAAGAAGTACGGCCTGCCCAACGACATCA |
| | | TTATCGAGCTGGCCCGCGAGAAGAACTCCAAGGACGCCCAGAAAATGATCA |
| | | ACGAGATGCAGAAGCGGAACCGGCAGACCAACGAGCGGATCGAGGAAATC |
| | | ATCCGGACCACCGGCAAAGAGAACGCCAAGTACCTGATCGAGAAGATCAA |
| | | GCTGCACGACATGCAGGAAGGCAAGTGCCTGTACAGCCTGGAAGCCATCCC |
| | | TCTGGAAGATCTGCTGAACAACCCCTTCAACTATGAGGTGGACCACATCAT |
| | | CCCCAGAAGCGTGTCCTTCGACAACAGCTTCAACAACAAGGTGCTCGTGAA |
| | | GCAGGAAGAAGCCAGCAAGAAGGGCAACCGGACCCCATTCCAGTACCTGA |
| | | GCAGCAGCGACAGCAAGATCAGCTACGAAACCTTCAAGAAGCACATCCTGA |
| | | ATCTGGCCAAGGGCAAGGGCAGAATCAGCAAGACCAAGAAAGAGTATCTG |
| | | CTGGAAGAACGGGACATCAACAGGTTCTCCGTGCAGAAAGACTTCATCAAC |
| | | CGGAACCTGGTGGATACCAGATACGCCACCAGAGGCCTGATGAACCTGCTG |
| | | CGGAGCTACTTCAGAGTGAACAACCTGGACGTGAAAGTGAAGTCCATCAAT |
| | | GGCGGCTTCACCAGCTTTCTGCGGCGGAAGTGGAAGTTTAAGAAAGAGCGG |
| | | AACAAGGGGTACAAGCACCACGCCGAGGACGCCCTGATCATTGCCAACGCC |
| | | GATTTCATCTTCAAAGAGTGGAAGAAACTGGACAAGGCCAAAAAAGTGATG |
| | | GAAAACCAGATGTTCGAGGAAAAGCAGGCCGAGAGCATGCCCGAGATCGA |
| | | AACCGAGCAGGAGTACAAAGAGATCTTCATCACCCCCCACCAGATCAAGCA |
| | | CATTAAGGACTTCAAGGACTACAAGTACAGCCACCGGGTGGACAAGAAGCC |
| | | TAATAGAGAGCTGATTAACGACACCCTGTACTCCACCCGGAAGGACGACAA |
| | | GGGCAACACCCTGATCGTGAACAATCTGAACGGCCTGTACGACAAGGACAA |
| | | TGACAAGCTGAAAAAGCTGATCAACAAGAGCCCCGAAAAGCTGCTGATGTA |
| | | CCACCACGACCCCCAGACCTACCAGAAACTGAAGCTGATTATGGAACAGTA |
| | | CGGCGACGAGAAGAATCCCCTGTACAAGTACTACGAGGAAACCGGGAACT |
| | | ACCTGACCAAGTACTCCAAAAAGGACAACGGCCCCGTGATCAAGAAGATTA |
| | | AGTATTACGGCAACAAACTGAACGCCCATCTGGACATCACCGACGACTACC |
| | | CCAACAGCAGAAACAAGGTCGTGAAGCTGTCCCTGAAGCCCTACAGATTCG |
| | | ACGTGTACCTGGACAATGGCGTGTACAAGTTCGTGACCGTGAAGAATCTGG |
| | | ATGTGATCAAAAAAGAAAACTACTACGAAGTGAATAGCAAGTGCTATGAG |
| | | GAAGCTAAGAAGCTGAAGAAGATCAGCAACCAGGCCGAGTTTATCGCCTCC |
| | | TTCTACAACAACGATCTGATCAAGATCAACGGCGAGCTGTATAGAGTGATC |
| | | GGCGTGAACAACGACCTGCTGAACCGGATCGAAGTGAACATGATCGACATC |
| | | ACCTACCGCGAGTACCTGGAAAACATGAACGACAAGAGGCCCCCCAGGATC |
| | | ATTAAGACAATCGCCTCCAAGACCCAGAGCATTAAGAAGTACAGCACAGAC |
| | | ATTCTGGGCAACCTGTATGAAGTGAAATCTAAGAAGCACCCTCAGATCATC |
| | | AAAAAGGGCAAAAGGCCGGCGGCCACGAAAAAGGCCGGCCAGGCAAAA |
| | | AAGAAAAAGGGATCCGAGGCCAGCGGTTCCGGACGGGCTGACGCATTGGA |
| | | CGATTTTGATCTGGATATGCTGGGAAGTGACGCCCTCGATGATTTTGACCTT |
| | | GACATGCTTGGTTCGGATGCCCTTGATGACTTTGACCTCGACATGCTCGGCA |
| | | GTGACGCCCTTGATGATTTCGACCTGGACATGCTGTGATCTAGAGAATTCCT |
| | | AGAGCTCGCTGATCAGCCTCGACTGTGCCTTCTAGTTGCCAGCCATCTGT |
| | | TGTTTGCCCCTCCCCCGTGCCTTCCTTGACCCTGGAAGGTGCCACTCCC |
| | | ACTGTCCTTTCCTAATAAAATGAGGAAATTGCATCGCATTGTCTGAGTA |
| | | GGTGTCATTCTATTCTGGGGGGTGGGGGGGGCAGGACAGCAAGGGG |
| | | GAGGATTGGGAAGAGAATAGCAGGCATGCTGGGGAgctagaggccgcaggaaccc |
| | | ctagtgatggagttggccactccctctctgcgcgctcgctcgctcactgagcccgggcgaccaaaggtcgcccgacccccg |
| | | ggcggcctcagtgagcgagcgagcgcgcagctgcctgcaggacatgtgagcaaaaggccagcaaaaggccaggaaccg |
| | | taaaaaggccgcgttgctggcgtttttccataggctccgcccccctgacgagcatcacaaaaatcgacgctcaagtcaga |
| | | ggtggcgaaacccgacaggactataaagataccaggcgtttccccctggaagctccctcgtgcgctctcctgttccgac |
| | | cctgccgcttaccggatacctgtccgcctttctcccttcgggaagcgtggcgctttctcatagctcacgctgtaggtatctc |
| | | agttcggtgtaggtcgttcgctccaagctgggctgtgtgcacgaaccccccgttcagcccgaccgctgcgccttatccggt |
| | | aactatcgtcttgagtccaacccggtaagacacgacttatcgccactggcagcagccactggtaacaggattagcagagcga |
| | | ggtatgtaggcggtgctacagagttcttgaagtggtggcctaactacggctacactagaagaacagtatttggtatctgcgctct |
| | | gctgaagccagttaccttcggaaaaagagttggtagctcttgatccggcaaacaaaccaccgctggtagcggtggtttttttgttt |
| | | gcaagcagcagattacgcgcagaaaaaaaggatctcaagaagatcctttgatctttctacggggtctgacgctcagtggaac |
| | | gaaaactcacgttaagggattttggtcatgagattatcaaaaaggatcttcacctagatccttttaaattaaaaatgaagttttaaat |
| | | caatctaaagtatatatgagtaaacttggtctgacagttaccaatgcttaatcagtgaggcacctatctcagcgatctgtctatttcg |
| | | ttcatccatagttgcctgactccccgtcgtgtagataactacgatacgggagggcttaccatctggccccagtcctccaatgata |
| | | ccgcggcttccacgctcaccggctccagatttatcagcaataaaccagccagccggaagggccgagcgcagaagtggtcct |
| | | gcaactttatccgcctccatccagtctattaattgttgccgggaagctagagtaagtagttcgccagttaatagtttgcgcaacgtt |
| | | gttgccattgctacaggcatcgtggtgtcacgctcgtcgtttggtatggcttcattcagctccggttcccaacgatcaaggcgagt |
| | | tacatgatcccccatgttgtgcaaaaagcggttagctccttcggtcctccgatcgttgtcagaagtaagttggccgcagtgttat |
| | | cactcatggttatgg |
| 15 | AAV-U6- | cagcactgcataattctcttactgtcatgccatccgtaagatgcttttctgtgactggtgagtactcaaccaagtcattctg |
| | Shank3- | agaatagtgtatgcggcgaccgagttgctcttgcccggcgtcaatacgggataataccgcgccacatagcagaacttt |
| | gRNA7-CMV- | aaaagtgctcatcattggaaaacgttcttcggggcgaaaactctcaaggatcttaccgctgttgagatccagttcgatgt |
| | dSaCas9-VP64 | aacccactcgtgcacccaactgatcttcagcatcttttactttcaccagcgtttctgggtgagcaaaaacaggaaggca |
| | 5′ part of | aaatgccgcaaaaaagggaataagggcgacacggaaatgttgaatactcatactcttcctttttcaatattattgaagcattt |
| | Ampicillin | atcaggcttattgtctcatgagcggatacatatttgaatgtatttagaaaaataaacaaataggggttccgcgcacatttccccga |
| | resistant gene | aaagtgccacctgacgtctaagaaaccattattatcatgacattaacctataaaaataggcgtatcacgaggccctttcgtctcgc |
| | (AmpR) (bold) | gcgtttcggtgatgacggtgaaaacctctgacacatgcagctcccggagacggtcacagcttgtctgtaagcggatgccggg |
| | AmpR promoter | agcagacaagcccgtcagggcgcgtcagcgggtgttgggggtgtcggggctggcttaactatgcggcatcagagcagatt |
| | (underlined)-f1 | gtactgagagtgcaccataaaattgtaaacgttaatattttgttaaaattcgcgttaaatttttgttaaatcagctcatttttt |
| | bacteriophage | aaccaatagaccgaaatcggcaaaatcccttataaatcaaaagaatagcccgagatagagttgagtgttgttccagtt |
| | origin of | tggaacaagagtccactattaaagaacgtggactccaacgtcaaagggcgaaaaaccgtctatcagggcgatggcc |
| | replication | cactacgtgaaccatcacccaaatcaagttttttggggtcgaggtgccgtaaagcactaaatcggaaccctaaagggagc |
| | (bold)-5′ITR | ccccgatttagagcttgacggggaaagccggcgaacgtggcgagaaaggaagggaagaaagcgaaaggagcgggcgct |
| | (underlined)-U6 | aaggcgctggcaagtgtagcggtcacgctgcgcgtaaccaccacacccgccgcgcttaatgcgccgctacagggcgcgta |
| | promoter | ctatggttgctttgacgtatgcggtgtgaaataccgcacagatgcgtaaggagaaaataccgcatcaggcgcccctgcaggca |
| | (bold)-Shank3 | gctgcgcgctcgctcgctcactgaggcccccgggcaaagcccgggcgtcgggcgacctttggtcgcccggcctcatga |
| | gRNA7 | gcgagcgagcgcgcgagagggagtggccaactccatcactaggggttcctgcggccgcctcgagGAGGGCCT |
| | (underlined)- | ATTTCCCATGATTCCTTCATATTTGCATATACGATACAAGGCTGTTAGA |
| | gRNA scaffold | GAGATAATTGGAATTAATTTGACTGTAAACACAAAGATATTAGTACAAA |
| | (bold)-CMV | ATACGTGACGTAGAAAGTAATAATTTCTTGGGTAGTTTGCAGTTTTAAA |
| | promoter | ATTATGTTTTAAAATGGACTATCATATGCTTACCGTAACTTGAAAGTAT |
| | (underlined) | TTCGATTTCTTGGCTTTATATATCTTGTGGAAAGGACGAAACACCGTCTC |
| | SV40 nuclear | TCCCTCTCAGTCGGCCTGTTATAGTACTCTGGAAACAGAATCTACTATAA |
| | localization | CAAGGCAAAATGCCGTGTTTATCTCGTCAACTTGTTGGCGAGATTTTTTT |
| | signal (bold)- | GCGTTGACATTGATTATTGACTAGTTATTAATAGTAATCAATTACGGGGTCA |
| | dSaCas9 | TTAGTTCATAGCCCATATATGGAGTTCCGCGTTACATAACTTACGGTAAATG |
| | (underlined)- | GCCCGCCTGGCTGACCGCCCAACGACCCCCGCCCATTGACGTCAATAATGA |
| | Nucleoplasmin | CGTATGTTCCCATAGTAACGCCAATAGGGACTTTCCATTGACGTCAATGGGT |
| | nuclear | GGAGTATTTACGGTAAACTGCCCACTTGGCAGTACATCAAGTGTATCATATG |
| | localization | CCAAGTACGCCCCCTATTGACGTCAATGACGGTAAATCGCCCGCCTCGCAT |
| | signal (bold)- | TATGCCCAGTACATGACCTTATGGGACTTTCCTACTTGGCAGTACATCTACG |
| | VP64 | TATTAGTCATCGCTATTACCATGGTGATGCGGTTTTGGCAGTACATCAATGG |
| | (underlined)- | GCGTGGATAGCGGTTTGACTCACGGGGATTTCCAAGTCTCCACCCCATTGAC |
| | Bovine growth | GTCAATGGGAGTTTGTTTTGGCACCAAAATCAACGGGACTTTCCAAAATGTC |
| | hormone | GTAACAACTCCGCCCCATTGACGCAAATGGGCGGTAGGCGTGTACGGTGGG |
| | polyadenylation | AGGTCTATATAAGCAGAGCTCTCTGGCTAACTACCGGTGCCACCATGGCCC |
| | signal (bold)- | CAAAGAAGAAGCGGAAGGTCGGTATCCACGGAGTCCCAGCAGCCAAGCC |
| | 3′ITR | GAACTACATCCTGGGCCTGGCCATCGGCATCACCAGCGTGGGCTACGGCAT |
| | (underlined)- | CATCGACTACGAGACACGGGACGTGATCGATGCCGGCGTGCGGCTGTTCAA |
| | high-copy- | AGAGGCCAACGTGGAAAACAACGAGGGCAGGCGGAGCAAGAGAGGCGCC |
| | number | AGAAGGCTGAAGCGGCGGAGGCGGCATAGAATCCAGAGAGTGAAGAAGCT |
| | ColE1/pMB1/ | GCTGTTCGACTACAACCTGCTGACCGACCACAGCGAGCTGAGCGGCATCAA |
| | pBR322/pUC | CCCCTACGAGGCCAGAGTGAAGGGCCTGAGCCAGAAGCTGAGCGAGGAAG |
| | origin of | AGTTCTCTGCCGCCCTGCTGCACCTGGCCAAGAGAAGAGGCGTGCACAACG |
| | replication | TGAACGAGGTGGAAGAGGACACCGGCAACGAGCTGTCCACCAAAGAGCAG |
| | (bold)-3′ part | ATCAGCCGGAACAGCAAGGCCCTGGAAGAGAAATACGTGGCCGAACTGCA |
| | of Ampicillin | GCTGGAACGGCTGAAGAAAGACGGCGAAGTGCGGGGCAGCATCAACAGAT |
| | resistant gene | TCAAGACCAGCGACTACGTGAAAGAAGCCAAACAGCTGCTGAAGGTGCAG |
| | (AmpR) | AAGGCCTACCACCAGCTGGACCAGAGCTTCATCGACACCTACATCGACCTG |
| | (underlined) | CTGGAAACCCGGCGGACCTACTATGAGGGACCTGGCGAGGGCAGCCCCTTC |
| | | GGCTGGAAGGACATCAAAGAATGGTACGAGATGCTGATGGGCCACTGCACC |
| | | TACTTCCCCGAGGAACTGCGGAGCGTGAAGTACGCCTACAACGCCGACCTG |
| | | TACAACGCCCTGAACGACCTGAACAATCTCGTGATCACCAGGGACGAGAAC |
| | | GAGAAGCTGGAATATTACGAGAAGTTCCAGATCATCGAGAACGTGTTCAAG |
| | | CAGAAGAAGAAGCCCACCCTGAAGCAGATCGCCAAAGAAATCCTCGTGAA |
| | | CGAAGAGGATATTAAGGGCTACAGAGTGACCAGCACCGGCAAGCCCGAGT |
| | | TCACCAACCTGAAGGTGTACCACGACATCAAGGACATTACCGCCCGGAAAG |
| | | AGATTATTGAGAACGCCGAGCTGCTGGATCAGATTGCCAAGATCCTGACCA |
| | | TCTACCAGAGCAGCGAGGACATCCAGGAAGAACTGACCAATCTGAACTCCG |
| | | AGCTGACCCAGGAAGAGATCGAGCAGATCTCTAATCTGAAGGGCTATACCG |
| | | GCACCCACAACCTGAGCCTGAAGGCCATCAACCTGATCCTGGACGAGCTGT |
| | | GGCACACCAACGACAACCAGATCGCTATCTTCAACCGGCTGAAGCTGGTGC |
| | | CCAAGAAGGTGGACCTGTCCCAGCAGAAAGAGATCCCCACCACCCTGGTGG |
| | | ACGACTTCATCCTGAGCCCCGTCGTGAAGAGAAGCTTCATCCAGAGCATCA |
| | | AAGTGATCAACGCCATCATCAAGAAGTACGGCCTGCCCAACGACATCATTA |
| | | TCGAGCTGGCCCGCGAGAAGAACTCCAAGGACGCCCAGAAAATGATCAAC |
| | | GAGATGCAGAAGCGGAACCGGCAGACCAACGAGCGGATCGAGGAAATCAT |
| | | CCGGACCACCGGCAAAGAGAACGCCAAGTACCTGATCGAGAAGATCAAGC |
| | | TGCACGACATGCAGGAAGGCAAGTGCCTGTACAGCCTGGAAGCCATCCCTC |
| | | TGGAAGATCTGCTGAACAACCCCTTCAACTATGAGGTGGACCACATCATCC |
| | | CCAGAAGCGTGTCCTTCGACAACAGCTTCAACAACAAGGTGCTCGTGAAGC |
| | | AGGAAGAAGCCAGCAAGAAGGGCAACCGGACCCCATTCCAGTACCTGAGC |
| | | AGCAGCGACAGCAAGATCAGCTACGAAACCTTCAAGAAGCACATCCTGAAT |
| | | CTGGCCAAGGGCAAGGGCAGAATCAGCAAGACCAAGAAAGAGTATCTGCT |
| | | GGAAGAACGGGACATCAACAGGTTCTCCGTGCAGAAAGACTTCATCAACCG |
| | | GAACCTGGTGGATACCAGATACGCCACCAGAGGCCTGATGAACCTGCTGCG |
| | | GAGCTACTTCAGAGTGAACAACCTGGACGTGAAAGTGAAGTCCATCAATGG |
| | | CGGCTTCACCAGCTTTCTGCGGCGGAAGTGGAAGTTTAAGAAAGAGCCGAA |
| | | CAAGGGGTACAAGCACCACGCCGAGGACGCCCTGATCATTGCCAACGCCGA |
| | | TTTCATCTTCAAAGAGTGGAAGAAACTGGACAAGGCCAAAAAAGTGATGGA |
| | | AAACCAGATGTTCGAGGAAAAGCAGGCCGAGAGCATGCCCGAGATCGAAA |
| | | CCGAGCAGGAGTACAAAGAGATCTTCATCACCCCCCACCAGATCAAGCACA |
| | | TTAAGGACTTCAAGGACTACAAGTACAGCCACCGGGTGGACAAGAAGCCTA |
| | | ATAGAGAGCTGATTAACGACACCCTGTACTCCACCCGGAAGGACGACAAGG |
| | | GCAACACCCTGATCGTGAACAATCTGAACGGCCTGTACGACAAGGACAATG |
| | | ACAAGCTGAAAAAGCTGATCAACAAGAGCCCCGAAAAGCTGCTGATGTACC |
| | | ACCACGACCCCCAGACCTACCAGAAACTGAAGCTGATTATGGAACAGTACG |
| | | GCGACGAGAAGAATCCCCTGTACAAGTACTACGAGGAAACCGGGAACTAC |
| | | CTGACCAAGTACTCCAAAAAGGACAACGGCCCCGTGATCAAGAAGATTAAG |
| | | TATTACGGCAACAAACTGAACGCCCATCTGGACATCACCGACGACTACCCC |
| | | AACAGCAGAAACAAGGTCGTGAAGCTGTCCCTGAAGCCCTACAGATTCGAC |
| | | GTGTACCTGGACAATGGCGTGTACAAGTTCGTGACCGTGAAGAATCTGGAT |
| | | GTGATCAAAAAAGAAAACTACTACGAAGTGAATAGCAAGTGCTATGAGGA |
| | | AGCTAAGAAGCTGAAGAAGATCAGCAACCAGGCCGAGTTTATCGCCTCCTT |
| | | CTACAACAACGATCTGATCAAGATCAACGGCGAGCTGTATAGAGTGATCGG |
| | | CGTGAACAACGACCTGCTGAACCGGATCGAAGTGAACATGATCGACATCAC |
| | | CTACCGCGAGTACCTGGAAAACATGAACGACAAGAGGCCCCCCAGGATCAT |
| | | TAAGACAATCGCCTCCAAGACCCAGAGCATTAAGAAGTACAGCACAGACAT |
| | | TCTGGGCAACCTGTATGAAGTGAAATCTAAGAAGCACCCTCAGATCATCAA |
| | | AAAGGGCAAAAGGCCGGCGGCCACGAAAAAGGCCGGCCAGGCAAAAAA |
| | | GAAAAAGGGATCCGAGGCCAGCGGTTCCGGACGGGCTGACGCATTCGACG |
| | | ATTTTGATCTGGATATGCTGGGAAGTGACGCCCTCGATGATTTTGACCTTGA |
| | | CATGCTTGGTTCGGATGCCCTTGATGACTTTGACCTCGACATGCTCGGCAGT |
| | | GACGCCCTTGATGATTTCGACCTGGACATGCTGTGATCTAGAGAATTCCTAG |
| | | AGCTCGCTGATCAGCCTCGACTGTGCCTTCTAGTTGCCAGCCATCTGTTG |
| | | TTTGCCCCTCCCCCGTGCCTTCCTTGACCCTGGAAGGTGCCACTCCCAC |
| | | TGTCCTTTCCTAATAAAATGAGGAAATTGCATCGCATTGTCTGAGTAGG |
| | | TGTCATTCTATTCTGGGGGGTGGGGTGGGGCAGGACAGCAAGGGGGA |
| | | GGATTGGGAAGAGAATAGCAGGCATGCTGGGGAgctagaggccgcaggaacccctag |
| | | tgatggagttggccactccctctctgcgcgctcgctcgctcactgaggccgggcgaccaaagctcccccgacgcccgcgcg |
| | | gcctcagtgagcgagcgagcgcgcagctgcctgcaggacatgtgagcaaaaggccagcaaaaggccaggaaccgtaaaa |
| | | aggccgcgttgctggcgtttttccataggctccgcccccctgacgagcatcacaaaaatcgacgctcaagtcagaggtg |
| | | gcgaaacccgacaggactataaagataccaggcgtttccccctggaagctccctcgtgcgctctcctgttccgaccctg |
| | | ccgcttaccggatacctgtccgcctttctcccttcgggaagcgtggcgctttctcatagctcacgctgtaggtatctcagtt |
| | | cggtgtaggtcgttcgctccaagctgggctgtgtgcacgaaccccccgttcagcccgaccgctgcgccttatccggtaact |
| | | atcgtcttgagtccaacccggtaagacacgacttatcgccactggcagcagccactggtaacaggattagcagagcgaggta |
| | | tgtaggcggtgctacagagttcttgaagtggtggcctaactacggctacactagaagaacagtatttggtatctgcgctctgctg |
| | | aagccagttaccttcggaaaaagagttggtagctcttgatccggcaaacaaaccaccgctggtagcggtggtttttttgtttgca |
| | | agcagcagattacgcgcagaaaaaaaggatctcaagaagatcctttgatcttttctacggggtctgacgctcagtggaacgaa |
| | | aactcacgttaagggattttggtcatgagattatcaaaaaggatcttcacctagatccttttaaattaaaaatgaagttttaaatcaat |
| | | ctaaagtatatatgagtaaacttggtctgacagttaccaatgcttaatcagtgaggcacctatctcagcgatctctctatttcgttca |
| | | tccatagttgcctgactccccgtcgtgtagataactacgatacgggagggcttaccatctggccccagtcctccaatgataccg |
| | | cggcttccacgctcaccggctccagatttatcagcaataaaccagccagccggaagggccgagcgcagaagtggtcctgca |
| | | actttatccgcctccatccagtctattaattgttgccgggaagctagagtaagtagttcgccagttaatagtttgcgcaacgttgttg |
| | | ccattgctacaggcatcgtggtgtcacgctcgtcgtttggtatggcttcattcagctccggttcccaacgatcaaggcgagttac |
| | | atgatcccccatgttgtgcaaaaaagcggttagctccttcggtcctccgatcgttgtcagaagtaagttggccgcagtgttatcac |
| | | tcatgcttatgg |
| 16 | AAV-U6- | cagcactgcataattctcttactgtcatgccatccgtaagatgcttttctgtgactggtgagtactcaaccaagtcattctg |
| | Control-hSyn- | agaatagtgtatgcggcgaccgagttgctcttgcccggcgtcaatacgggataataccgcgccacatagcagaacttt |
| | dSaCas9-VP64 | aaaagtgctcatcattggaaaacgttcttcggggcgaaaactctcaaggatcttaccgctgttgagatccagttcgatgt |
| | 5′ part of | aacccactcgtgcacccaactgatcttcagcatcttttactttcaccagcgtttctgggtgagcaaaaacaggaaggca |
| | Ampicillin | aaatgccgcaaaaaagggaataagggcgacacggaaatgttgaatactcatactcttcctttttcaatattattgaagcattt |
| | resistant gene | atcaggcttattctctcatgagcggatacatatttgaatctatttagaaaaataaacaaatagggcttccgcgcacatttccccga |
| | (AmpR) (bold)- | aaagtgccacctgacgtctaagaaaccattattatcatgacattaacctataaaaataggcgtatcacgaggccctttcgtctcgc |
| | AmpR promoter | gcgtttcggtgatgacggtgaaaacctctgacacatgcagctcccggagacggtcacagcttgtctgtaagcggatgccggg |
| | (underlined)-f1 | agcagacaagcccgtcagggcgcgtcagcgggtgttggcgggtgtggggctggcttaactatgcggcatcagagcagatt |
| | bacteriophage | gtactgagagtgcaccataaaattgtaaacgttaatattttgttaaaattcgcgttaaatttttgttaaatcagctcatttttt |
| | origin of | aaccaatagaccgaaatcggcaaaatcccttataaatcaaaagaatagcccgagatagagttgagtgttgttccagtt |
| | replication | tggaacaagagtccactattaaagaacgtggactccaacgtcaaagggcgaaaaaccgtctatcagggcgatggcc |
| | (bold)-5′TTR | cactacgtgaaccatcacccaaatcaagttttttggggtcgaggtgccgtaaagcactaaatcggaaccctaaagggagc |
| | (underlined)- | ccccgatttagagcttgacggggaaagccggcgaacgtggcgagaaaggaagggaagaaagcgaaaggagcgggcgct |
| | U6 promoter | aaggcgctggcaagtgtagcggtcacgctgcgcgtaaccaccacacccgccgcgcttaatgcgccgctacagggcgcgta |
| | (bold)-gRNA | ctatggttgctttgacgtatgcggtgtgaaataccgcacagatgcgtaaggagaaaataccgcatcaggcgcccctgcaggca |
| | scaffold | gctgcgcgctcgctcgctcactgaggccgcccgggcaaagcccgggcctccggcgacctttgctcgcccggcctcagtga |
| | (underlined)- | gcgagcgagcgcgcagagagggactggccaactccatcactagcgcttcctgcggccgcctcgagGAGGGCCT |
| | Human | ATTTCCCATGATTCCTTCATATTTGCATATACGATACAAGGCTGTTAGA |
| | Synapsin1 | GAGATAATTGGAATTAATTTGACTGTAAACACAAAGATATTAGTACAAA |
| | promoter (bold)- | ATACGTGACGTAGAAAGTAATAATTTCTTGGGTAGTTTGCAGTTTTAAA |
| | SV40 nuclear | ATTATGTTTTAAAATGGACTATCATATGCTTACCGTAACTTGAAAGTAT |
| | localization | TTCGATTTCTTGGCTTTATATATCTTGTGGAAAGGACGAAACACCGGAG |
| | signal | ACCACGGCAGGTCTCAGTTATAGTACTCTGGAAACAGAATCTACTATAACA |
| | (underlined)- | AGGCAAAATGCCGTGTTTATCTCGTCAACTTGTTGGCGAGATTTTTTTGCGT |
| | dSaCas9 (bold) | TGACATTGATTATTGACTAGTGTGTCTAGACTGCAGAGGGCCCTGCGTAT |
| | Nucleoplasmin | GAGTGCAAGTGGGTTTTAGGACCAGGATGAGGCGGGGGGGGGTGCC |
| | nuclear | TACCTGACGACCGACCCCGACCCACTGGACAAGCACCCAACCCCCATT |
| | localization | CCCCAAATTGCGCATCCCCTATCAGAGAGGGGGAGGGGAAACAGGATG |
| | signal | CGGCGAGGCGCGTGCGCACTGCCAGCTTCAGCACCGCGGACAGTGCCT |
| | (underlined)- | TCGCCCCCGCCTGGCGGCGCGCGCCACCGCCGCCTCAGCACTGAAGGC |
| | VP64 (bold)- | GCGCTGACGTCACTCGCCGGTCCCCCGCAAACTCCCCTTCCCGGCCAC |
| | Bovine growth | CTTGGTCGCGTCCGCGCCGCCGCCGGCCCAGCCGGACCGCACCACGCG |
| | hormone | AGGCGCGAGATAGGGGGGCACGGGCGCGACCATCTGCGCTGCGGCGC |
| | polyadenylation | CGGCGACTCAGCGCTGCCTCAGTCTGCGGTGGGCAGCGGAGGAGTCGT |
| | signal | GTCGTGCCTGAGAGCGCAGTCGAGAAACCGGCTAGAACCGGTGCCACC |
| | (underlined)- | ATGGCCCCAAAGAAGAAGCGGAAGGTCGGTATCCACGGAGTCCCAGCAGC |
| | 3′ITR (bold)- | CAAGCGGAACTACATCCTGGGCCTGGCCATCGGCATCACCAGCGTGGG |
| | high-copy- | CTACGGCATCATCGACTACGAGACACGGGACGTGATCGATGCCGGCGT |
| | number | GCGGCTGTTCAAAGAGGCCAACGTGGAAAACAACGAGGGCAGGCGGA |
| | ColE1/pMB1/ | GCAAGAGAGGCGCCAGAAGGCTGAAGCGGCGGAGGCGGCATAGAATC |
| | pBR322/pUC | CAGAGAGTGAAGAAGCTGCTGTTCGACTACAACCTGCTGACCGACCAC |
| | origin of | AGCGAGCTGAGCGGCATCAACCCCTACGAGGCCAGAGTGAAGGGCCTG |
| | replication | AGCCAGAAGCTGAGCGAGGAAGAGTTCTCTGCCGCCCTGCTGCACCTG |
| | (underlined)-3′ | GCCAAGAGAAGAGGCGTGCACAACGTGAACGAGGTGGAAGAGGACAC |
| | part of | CGGCAACGAGCTGTCCACCAAAGAGCAGATCAGCCGGAACAGCAAGGC |
| | Ampicillin | CCTGGAAGAGAAATACGTGGCCGAACTGCAGCTGGAACGGCTGAAGAA |
| | resistant gene | AGACGGCGAAGTGCGGGGCAGCATCAACAGATTCAAGACCAGCGACTA |
| | (AmpR) (bold) | CGTGAAAGAAGCCAAACAGCTGCTGAAGGTGCAGAAGGCCTACCACCA |
| | | GCTGGACCAGAGCTTCATCGACACCTACATCGACCTGCTGGAAACCCG |
| | | GCGGACCTACTATGAGGGACCTGGCGAGGGCAGCCCCTTCGGCTGGAA |
| | | GGACATCAAAGAATGGTACGAGATGCTGATGGGCCACTGCACCTACTT |
| | | CCCCGAGGAACTGCGGAGCGTGAAGTACGCCTACAACGCCGACCTGTA |
| | | CAACGCCCTGAACGACCTGAACAATCTCGTGATCACCAGGGACGAGAA |
| | | CGAGAAGCTGGAATATTACGAGAAGTTCCAGATCATCGAGAACGTGTT |
| | | CAAGCAGAAGAAGAAGCCCACCCTGAAGCAGATCGCCAAAGAAATCCT |
| | | CGTGAACGAAGAGGATATTAAGGGCTACAGAGTGACCAGCACCGGCAA |
| | | GCCCGAGTTCACCAACCTGAAGGTGTACCACGACATCAAGGACATTAC |
| | | CGCCCGGAAAGAGATTATTGAGAACGCCGAGCTGCTGGATCAGATTGC |
| | | CAAGATCCTGACCATCTACCAGAGCAGCGAGGACATCCAGGAAGAACT |
| | | GACCAATCTGAACTCCGAGCTGACCCAGGAAGAGATCGAGCAGATCTC |
| | | TAATCTGAAGGGCTATACCGGCACCCACAACCTGAGCCTGAAGGCCAT |
| | | CAACCTGATCCTGGACGAGCTGTGGCACACCAACGACAACCAGATCGC |
| | | TATCTTCAACCGGCTGAAGCTGGTGCCCAAGAAGGTGGACCTGTCCCA |
| | | GCAGAAAGAGATCCCCACCACCCTGGTGGACGACTTCATCCTGAGCCC |
| | | CGTCGTGAAGAGAAGCTTCATCCAGAGCATCAAAGTGATCAACGCCAT |
| | | CATCAAGAAGTACGGCCTGCCCAACGACATCATTATCGAGCTGGCCCG |
| | | CGAGAAGAACTCCAAGGACGCCCAGAAAATGATCAACGAGATGCAGAA |
| | | GCGGAACCGGCAGACCAACGAGCGGATCGAGGAAATCATCCGGACCAC |
| | | CGGCAAAGAGAACGCCAAGTACCTGATCGAGAAGATCAAGCTGCACGA |
| | | CATGCAGGAAGGCAAGTGCCTGTACAGCCTGGAAGCCATCCCTCTGGA |
| | | AGATCTGCTGAACAACCCCTTCAACTATGAGGTGGACCACATCATCCCC |
| | | AGAAGCGTGTCCTTCGACAACAGCTTCAACAACAAGGTGCTCGTGAAG |
| | | CAGGAAGAAGCCAGCAAGAAGGGCAACCGGACCCCATTCCAGTACCTG |
| | | AGCAGCAGCGACAGCAAGATCAGCTACGAAACCTTCAAGAAGCACATC |
| | | CTGAATCTGGCCAAGGGCAAGGGCAGAATCAGCAAGACCAAGAAAGAG |
| | | TATCTGCTGGAAGAACGGGACATCAACAGGTTCTCCGTGCAGAAAGAC |
| | | TTCATCAACCGGAACCTGGTGGATACCAGATACGCCACCAGAGGCCTG |
| | | ATGAACCTGCTGCGGAGCTACTTCAGAGTGAACAACCTGGACGTGAAA |
| | | GTGAAGTCCATCAATGGCGGCTTCACCAGCTTTCTGCGGCGGAAGTGG |
| | | AAGTTTAAGAAAGAGCGGAACAAGGGGTACAAGCACCACGCCGAGGAC |
| | | GCCCTGATCATTGCCAACGCCGATTTCATCTTCAAAGAGTGGAAGAAAC |
| | | TGGACAAGGCCAAAAAAGTGATGGAAAACCAGATGTTCGAGGAAAAGC |
| | | AGGCCGAGAGCATGCCCGAGATCGAAACCGAGCAGGAGTACAAAGAG |
| | | ATCTTCATCACCCCCCACCAGATCAAGCACATTAAGGACTTCAAGGACT |
| | | ACAAGTACAGCCACCGGGTGGACAAGAAGCCTAATAGAGAGCTGATTA |
| | | ACGACACCCTGTACTCCACCCGGAAGGACGACAAGGGCAACACCCTGA |
| | | TCGTGAACAATCTGAACGGCCTGTACGACAAGGACAATGACAAGCTGA |
| | | AAAAGCTGATCAACAAGAGCCCCGAAAAGCTGCTGATGTACCACCACG |
| | | ACCCCCAGACCTACCAGAAACTGAAGCTGATTATGGAACAGTACGGCG |
| | | ACGAGAAGAATCCCCTGTACAAGTACTACGAGGAAACCGGGAACTACC |
| | | TGACCAAGTACTCCAAAAAGGACAACGGCCCCGTGATCAAGAAGATTA |
| | | AGTATTACGGCAACAAACTGAACGCCCATCTGGACATCACCGACGACT |
| | | ACCCCAACAGCAGAAACAAGGTCGTGAAGCTGTCCCTGAAGCCCTACA |
| | | GATTCGACGTGTACCTGGACAATGGCGTGTACAAGTTCGTGACCGTGA |
| | | AGAATCTGGATGTGATCAAAAAAGAAAACTACTACGAAGTGAATAGCA |
| | | AGTGCTATGAGGAAGCTAAGAAGCTGAAGAAGATCAGCAACCAGGCCG |
| | | AGTTTATCGCCTCCTTCTACAACAACGATCTGATCAAGATCAACGGCGA |
| | | GCTGTATAGAGTGATCGGCGTGAACAACGACCTGCTGAACCGGATCGA |
| | | AGTGAACATGATCGACATCACCTACCGCGAGTACCTGGAAAACATGAA |
| | | CGACAAGAGGCCCCCCAGGATCATTAAGACAATCGCCTCCAAGACCCA |
| | | GAGCATTAAGAAGTACAGCACAGACATTCTGGGCAACCTGTATGAAGT |
| | | GAAATCTAAGAAGCACCCTCAGATCATCAAAAAGGGCAAAAGGCCGGCG |
| | | GCCACGAAAAAGGCCGGCCAGGCAAAAAAGAAAAAGGGATCCGAGGCCAG |
| | | CGGTTCCGGACGGGCTGACGCATTGGACGATTTTGATCTGGATATGCTG |
| | | GGAAGTGACGCCCTCGATGATTTTGACCTTGACATGCTTGGTTCGGAT |
| | | GCCCTTGATGACTTTGACCTCGACATGCTCGGCAGTGACGCCCTTGAT |
| | | GATTTCGACCTGGACATGCTGTGATCTAGAGAATTCCTAGAGCTCGCTGA |
| | | TCAGCCTCGACTGTGCCTTCTAGTTGCCAGCCATCTGTTGTTTGCCCCTCCCC |
| | | CGTGCCTTCCTTGACCCTGGAAGGTGCCACTCCCACTGTCCTTTCCTAATAA |
| | | AATGAGGAAATTGCATCGCATTGTCTGAGTAGGTGTCATTCTATTCTGGGGG |
| | | GTGGGGGGGGCAGGACAGCAAGGGGGAGGATTGGGAAGAGAATAGCAGG |
| | | CATGCTGGGGAgctagaggccgcaggaacccctagtgatggagttggccactccctctctgcgcgctcgctcg |
| | | ctcactgaggccgggcgaccaaaggtcgcccgacgcccgggcggcctcagtgagcgagcgagcgcgcagctgcctg |
| | | caggacatgtgagcaaaaggccagcaaaaggccaggaaccgtaaaaaggccgcgttgctggcgtttttccataggctccgc |
| | | ccccctgacgagcatcacaaaaalccaccctcaagtcagaggtgccgaaaccccacaggactataaagataccagccgttt |
| | | ccccctggaagctccctcgtgcgctctcctgttccgaccctcccgcttaccggatacctgtccgcctttctcccttcgggaagcg |
| | | tggcgctttctcatagctcacgctgtaggtatctcagttcggtgtaggtcgttcgctccaagctgggctgtgtgcacgaaccccc |
| | | cgttcagcccgaccgctgcgccttatccggtaactatcgtcttgagtccaacccggtaagacacgacttatcgccactggcagc |
| | | agccactggtaacaggattagcagagcgaggtatgtaggcggtgctacagagttcttgaagtggtggcctaactacggctaca |
| | | ctagaagaacagtatttggtatctgcgctctgctgaagccagttaccttcggaaaaagagttggtagctcttgatccggcaaaca |
| | | aaccaccgctggtagcggtggtttttttgtttgcaagcagcagattacgcgcagaaaaaaaggatctcaagaagatcctttgatc |
| | | ttttctacggggtctgacgctcagtggaacgaaaactcacgttaagggattttggtcatgagattatcaaaaaggatcttcaccta |
| | | gatccttttaaattaaaaatgaagttttaaatcaatctaaagtatatatgagtaaacttggtctgacagttaccaatgcttaatcagt |
| | | gaggcacctatctcagcgatctgtctatttcgttcatccatagttgcctgactccccgtcgtgtagataactacgatacgg |
| | | gagggcttaccatctggccccagtgctgcaatgataccgcggcttccacgctcaccggctccagatttatcagcaataa |
| | | accagccagccggaagggccgagcgcagaagtggtcctgcaactttatccgcctccatccagtctattaattgttgccg |
| | | ggaagctagagtaagtagttcgccagttaatagtttgcgcaacgttgttgccattgctacaggcatcgtggtgtcacgct |
| | | cgtcgtttggtatggcttcattcagctccggttcccaacgatcaaggcgagttacatgatcccccatgtgtgcaaaaaa |
| | | gcggttagctccttcggtcctccgatcgttgtcagaagtaagttggccgcagtgttatcactcatggttatgg |
| 17 | AAV-U6- | cagcactgcataattctcttactgtcatgccatccgtaagatgcttttctgtgactggtgagtactcaaccaagtcattctg |
| | Shank3- | agaatagtgtatgcggcgaccgagttgctcttgcccggcgtcaatacgggataataccgcgccacatagcagaacttt |
| | gRNA3-hSyn- | aaaagtgctcatcattggaaaacgttcttcggggcgaaaactctcaaggatcttaccgctgttgagatccagttcgatgt |
| | dSaCas9-VP64 | aacccactcgtgcacccaactgatcttcagcatcttttactttcaccagcgtttctgggtgagcaaaaacaggaaggca |
| | 5′ part of | aaatgccgcaaaaaagggaataagggcgacacggaaatgttgaatactcatactcttcctttttcaatattattgaagcattt |
| | Ampicillin | atcagggttattgtctcatgagcggatacatatttgaatgtatttagaaaaataaacaaataggggttccgcgcacatttccccga |
| | resistant gene | aaagtgccacctgacgtctaagaaaccattattatcatgacattaacctataaaaataggcgtatcacgaggccctttcgtctcgc |
| | (AmpR) (bold)- | gcgtttcggtgatgacggtgaaaacctctgacacatgcagctcccggagacggtcacagcttgtctgtaagcggatgccggg |
| | AmpR promoter | agcagacaagcccgtcagggcgcgtcagcgggtgttggcgggtgtcggggctggcttaactatgcggcatcagagcagatt |
| | (underlined)-f1 | gtactgagagtgcaccataaaattgtaaacgttaatattttgttaaaattcgcgttaaatttttgttaaatcagctcatttttt |
| | bacteriophage | aaccaatagaccgaaatcggcaaaatcccttataaatcaaaagaatagcccgagatagagttgagtgttgttccagtt |
| | origin of | tggaacaagagtccactattaaagaacgtggactccaacgtcaaagggcgaaaaaccgtctatcagggcgatggcc |
| | replication | cactacgtgaaccatcacccaaatcaagttttttggggtcgaggtgccgtaaagcactaaatcggaaccctaaagggagc |
| | (bold)-5′ITR | ccccgatttagagcttgacggggaaagccggcgaacgtggcgagaaaggaagggaagaaagcgaaaggagcgggcgct |
| | (underlined)- | aaggcgctggcaagtgtagcggtcacgctgcgcgtaaccaccacacccgccgcgcttaatgcgccgctacagggcgcgta |
| | U6 promoter | ctatggttgctttgacgtatgcggtgtgaaataccgcacagatgcgtaaggagaaaataccgcatcaggcgcccctgcaggca |
| | (bold)-Shank3 | gctgcgcgctccctcgctcactgaggcccccccgccaaagcccgggcgtcgggcgacctttggtcgcccggcctcagtga |
| | gRNA3 | gcgagcgagcgcccagagagggagtggccaactccatcactaggggttcctgcggccgcctcgagGAGGGCCT |
| | (underlined)- | ATTTCCCATGATTCCTTCATATTTGCATATACGATACAAGGCTGTTAGA |
| | gRNA scaffold | GAGATAATTGGAATTAATTTGACTGTAAACACAAAGATATTAGTACAAA |
| | (bold)-Human | ATACGTGACGTAGAAAGTAATAATTTCTTGGGTAGTTTGCAGTTTTAAA |
| | Synapsin1 | ATTATGTTTTAAAATGGACTATCATATGCTTACCGTAACTTGAAAGTAT |
| | promoter | TTCGATTTCTTGGCTTTATATATCTTGTGGAAAGGACGAAACACCGCCG |
| | (underlined)- | GGAAACTGGGCTCCCCCGGTTATAGTACTCTGGAAACAGAATCTACTATA |
| | SV40 nuclear | ACAAGGCAAAATGCCGTGTTTATCTCGTCAACTTGTTGGCGAGATTTTT |
| | localization | TTGCGTTGACATTGATTATTGACTAGTGTGTCTAGACTGCAGAGGGCCCTGC |
| | signal (bold)- | GTATGAGTGCAAGTGGGTTTTAGGACCAGGATGAGGGGGGTGGGGGTGCC |
| | dSaCas9 | TACCTGACGACCGACCCCGACCCACTGGACAAGCACCCAACCCCCATTCCC |
| | (underlined)- | CAAATTGCGCATCCCCTATCAGAGAGGGGGAGGGGAAACAGGATGCGGCG |
| | Nucleoplasmin | AGGCGCGTGCGCACTGCCAGCTTCAGCACCGCGGACAGTGCCTTCGCCCCC |
| | nuclear | GCCTGGCGGCGCGCGCCACCGCCGCCTCAGCACTGAAGGCGCGCTGACGTC |
| | localization | ACTCGCCGGTCCCCCGCAAACTCCCCTTCCCGGCCACCTTGGTCGCGTCCGC |
| | signal (bold)- | GCCGCCGCCGGCCCAGCCGGACCGCACCACGCGAGGCGCGAGATAGGGGG |
| | VP64 | GCACGGGCGCGACCATCTGCGCTGCGGCGCCGGCGACTCAGCGCTGCCTCA |
| | (underlined)- | GTCTGCGGTGGGCAGCGGAGGAGTCGTGTCGTGCCTGAGAGCGCAGTCGAG |
| | Bovine growth | AAACCGGCTAGAACCGGTGCCACCATGGCCCCAAAGAAGAAGCGGAAGG |
| | hormone | TCGGTATCCACGGAGTCCCAGCAGCCAAGCGGAACTACATCCTGGGCCTGG |
| | polyadenylation | CCATCGGCATCACCAGCGTGGGCTACGGCATCATCGACTACGAGACACGGG |
| | signal (bold)- | ACGTGATCGATGCCGGCGTGCGGCTGTTCAAAGAGGCCAACGTGGAAAACA |
| | 3′ITR | ACGAGGGCAGGCGGAGCAAGAGAGGCGCCAGAAGGCTGAAGCGGCGGAG |
| | (underlined)- | GCGGCATAGAATCCAGAGAGTGAAGAAGCTGCTGTTCGACTACAACCTGCT |
| | high-copy- | GACCGACCACAGCGAGCTGAGCGGCATCAACCCCTACGAGGCCAGAGTGA |
| | number | AGGGCCTGAGCCAGAAGCTGAGCGAGGAAGAGTTCTCTGCCGCCCTGCTGC |
| | ColE1/pMB1/ | ACCTGGCCAAGAGAAGAGGCGTGCACAACGTGAACGAGGTGGAAGAGGAC |
| | pBR322/pUC | ACCGGCAACGAGCTGTCCACCAAAGAGCAGATCAGCCGGAACAGCAAGGC |
| | origin of | CCTGGAAGAGAAATACGTGGCCGAACTGCAGCTGGAACGGCTGAAGAAAG |
| | replication | ACGGCGAAGTGCGGGGCAGCATCAACAGATTCAAGACCAGCGACTACGTG |
| | (bold)-3′ part | AAAGAAGCCAAACAGCTGCTGAAGGTGCAGAAGGCCTACCACCAGCTGGA |
| | of Ampicillin | CCAGAGCTTCATCGACACCTACATCGACCTGCTGGAAACCCGGCGGACCTA |
| | resistant gene | CTATGAGGGACCTGGCGAGGGCAGCCCCTTCGGCTGGAAGGACATCAAAGA |
| | (AmpR) | ATGGTACGAGATGCTGATGGGCCACTGCACCTACTTCCCCGAGGAACTGCG |
| | (underlined) | GAGCGTGAAGTACGCCTACAACGCCGACCTGTACAACGCCCTGAACGACCT |
| | | GAACAATCTCGTGATCACCAGGGACGAGAACGAGAAGCTGGAATATTACG |
| | | AGAAGTTCCAGATCATCGAGAACGTGTTCAAGCAGAAGAAGAAGCCCACCC |
| | | TGAAGCAGATCGCCAAAGAAATCCTCGTGAACGAAGAGGATATTAAGGGCT |
| | | ACAGAGTGACCAGCACCGGCAAGCCCGAGTTCACCAACCTGAAGGTGTACC |
| | | ACGACATCAAGGACATTACCGCCCGGAAAGAGATTATTGAGAACGCCGAGC |
| | | TGCTGGATCAGATTGCCAAGATCCTGACCATCTACCAGAGCAGCGAGGACA |
| | | TCCAGGAAGAACTGACCAATCTGAACTCCGAGCTGACCCAGGAAGAGATCG |
| | | AGCAGATCTCTAATCTGAAGGGCTATACCGGCACCCACAACCTGAGCCTGA |
| | | AGGCCATCAACCTGATCCTGGACGAGCTGTGGCACACCAACGACAACCAGA |
| | | TCGCTATCTTCAACCGGCTGAAGCTGGTGCCCAAGAAGGTGGACCTGTCCC |
| | | AGCAGAAAGAGATCCCCACCACCCTGGTGGACGACTTCATCCTGAGCCCCG |
| | | TCGTGAAGAGAAGCTTCATCCAGAGCATCAAAGTGATCAACGCCATCATCA |
| | | AGAAGTACGGCCTGCCCAACGACATCATTATCGAGCTGGCCCGCGAGAAGA |
| | | ACTCCAAGGACGCCCAGAAAATGATCAACGAGATGCAGAAGCGGAACCGG |
| | | CAGACCAACGAGCGGATCGAGGAAATCATCCGGACCACCGGCAAAGAGAA |
| | | CGCCAAGTACCTGATCGAGAAGATCAAGCTGCACGACATGCAGGAAGGCA |
| | | AGTGCCTGTACAGCCTGGAAGCCATCCCTCTGGAAGATCTGCTGAACAACC |
| | | CCTTCAACTATGAGGTGGACCACATCATCCCCAGAAGCGTGTCCTTCGACA |
| | | ACAGCTTCAACAACAAGGTGCTCGTGAAGCAGGAAGAAGCCAGCAAGAAG |
| | | GGCAACCGGACCCCATTCCAGTACCTGAGCAGCAGCGACAGCAAGATCAGC |
| | | TACGAAACCTTCAAGAAGCACATCCTGAATCTGGCCAAGGGCAAGGGCAGA |
| | | ATCAGCAAGACCAAGAAAGAGTATCTGCTGGAAGAACGGGACATCAACAG |
| | | GTTCTCCGTGCAGAAAGACTTCATCAACCGGAACCTGGTGGATACCAGATA |
| | | CGCCACCAGAGGCCTGATGAACCTGCTGCGGAGCTACTTCAGAGTGAACAA |
| | | CCTGGACGTGAAAGTGAAGTCCATCAATGGCGGCTTCACCAGCTTTCTGCG |
| | | GCGGAAGTGGAAGTTTAAGAAAGAGCGGAACAAGGGGTACAAGCACCACG |
| | | CCGAGGACGCCCTGATCATTGCCAACGCCGATTTCATCTTCAAAGAGTGGA |
| | | AGAAACTGGACAAGGCCAAAAAAGTGATGGAAAACCAGATGTTCGAGGAA |
| | | AAGCAGGCCGAGAGCATGCCCGAGATCGAAACCGAGCAGGAGTACAAAGA |
| | | GATCTTCATCACCCCCCACCAGATCAAGCACATTAAGGACTTCAAGGACTA |
| | | CAAGTACAGCCACCGGGTGGACAAGAAGCCTAATAGAGAGCTGATTAACG |
| | | ACACCCTGTACTCCACCCGGAAGGACGACAAGGGCAACACCCTGATCGTGA |
| | | ACAATCTGAACGGCCTGTACGACAAGGACAATGACAAGCTGAAAAAGCTG |
| | | ATCAACAAGAGCCCCGAAAAGCTGCTGATGTACCACCACGACCCCCAGACC |
| | | TACCAGAAACTGAAGCTGATTATGGAACAGTACGGCGACGAGAAGAATCCC |
| | | CTGTACAAGTACTACGAGGAAACCCGGAACTACCTGACCAAGTACTCCAAA |
| | | AAGGACAACGGCCCCGTGATCAAGAAGATTAAGTATTACGGCAACAAACTG |
| | | AACGCCCATCTGGACATCACCGACGACTACCCCAACAGCAGAAACAAGGTC |
| | | GTGAAGCTGTCCCTGAAGCCCTACAGATTCGACGTGTACCTGGACAATGGC |
| | | GTGTACAAGTTCGTGACCGTGAAGAATCTGGATGTGATCAAAAAAGAAAAC |
| | | TACTACGAAGTGAATAGCAAGTGCTATGAGGAAGCTAAGAAGCTGAAGAA |
| | | GATCAGCAACCAGGCCGAGTTTATCGCCTCCTTCTACAACAACGATCTGATC |
| | | AAGATCAACGGCGAGCTGTATAGAGTGATCGGCGTGAACAACGACCTGCTG |
| | | AACCGGATCGAAGTGAACATGATCGACATCACCTACCGCGAGTACCTGGAA |
| | | AACATGAACGACAAGAGGCCCCCCAGGATCATTAAGACAATCGCCTCCAAG |
| | | ACCCAGAGCATTAAGAAGTACAGCACAGACATTCTGGGCAACCTGTATGAA |
| | | GTGAAATCTAAGAAGCACCCTCAGATCATCAAAAAGGGCAAAAGGCCGGC |
| | | GGCCACGAAAAAGGCCGGCCAGGCAAAAAAGAAAAAGGGATCCGAGGC |
| | | CAGCGGTTCCGGACGGGCTGACGCATTGGACGATTTTGATCTGGATATGCT |
| | | GGGAAGTGACGCCCTCGATGATTTTGACCTTGACATGCTTGGTTCGGATGCC |
| | | CTTGATGACTTTGACCTCGACATGCTCGGCAGTGACGCCCTTGATGATTTCG |
| | | ACCTGGACATGCTGTGATCTAGAGAATTCCTAGAGCTCGCTGATCAGCCTCG |
| | | ACTGTGCCTTCTAGTTGCCAGCCATCTGTTGTTTGCCCCTCCCCCGTGC |
| | | CTTCCTTGACCCTGGAAGGTGCCACTCCCACTGTCCTTTCCTAATAAAA |
| | | TGAGGAAATTGCATCGCATTGTCTGAGTAGGTGTCATTCTATTCTGGGG |
| | | GGTGGGGGGGGCAGGACAGCAAGGGGGAGGATTGGGAAGAGAATAG |
| | | CAGGCATGCTGGGGAgctagaggccgcaggaacccctagtgatggagttgcccactccctctctccccgct |
| | | cgctcgctcactgagcccgggcgaccaaaggtcgcccgaccccccggggggcctcagtgagccagcgagcccccagctg |
| | | cctgcaggacatgtgagcaaaaggccagcaaaaggccaggaaccgtaaaaaggccgcgttgctggcgtttttccataggct |
| | | ccgcccccctgacgagcatcacaaaaatcgacgctcaagtcagaggtggcgaaacccgacaggactataaagatac |
| | | caggcgtttccccctggaagctccctcgtgcgctctcctgttccgaccctgccgcttaccggatacctgtccgcctttctcc |
| | | cttcgggaagcgtggcgctttctcatagctcacgctgtaggtatctcagttcggtgtaggtcgttcgctccaagctgggct |
| | | gtgtgcacgaaccccccgttcagcccgaccgctgcgccttatccggtaactatcgtcttgagtccaacccggtaagacacgac |
| | | ttatcgccactggcagcagccactggtaacaggattagcagagcgaggtatgtaggcggtgctacagagttcttgaagtggtg |
| | | gcctaactacggctacactagaagaacagtatttggtatctgcgctctgctgaagccagttaccttcggaaaaagagttggtag |
| | | ctcttgatccggcaaacaaaccaccgctggtagcggtggtttttttgtttgcaagcagcagattacgcgcagaaaaaaaggatc |
| | | tcaagaagatcctttgatcttttctacggggtctgacgctcagtggaacgaaaactcacgttaagggattttggtcatgagattatc |
| | | aaaaaggatcttcacctagatccttttaaattaaaaatgaagttttaaatcaatctaaagtatatatgagtaaacttggtctgacagtt |
| | | accaatccttaatcagtgaggcacctatctcagcgatctgtctatttcgttcatccatagttgcctgactccccgtcgtgtagataa |
| | | ctacgatacgggagggcttaccatctggccccagtgctgcaatgataccgcggcttccacgctcaccggctccagatttatca |
| | | gcaataaaccagccagccggaagggccgagcgcagaagtcctcctgcaactttatccgcctccatccagtctattaattcttg |
| | | ccgggaagctagagtaagtagttcgccagttaatagtttgcgcaacgttgttgccattgctacaggcatcgtggtgtcacgctcg |
| | | tcgtttggtatgccttcattcagctcccgttcccaacgatgcgagttacatgatcccccatgttgtccaaaaaagcggttag |
| | | ctccttcggtcctccgatcgttctcagaagtaagttggccgcagtgttatcactcatggttatgg |
| 18 | U6 promoter | gagggcctatttcccatgattccttcatatttgcatatacgatacaaggctgttagagagataattggaattaatttgactgtaaaca |
| | | caaagatattagtacaaaatacgtgacgtagaaagtaataatttcttgggtagtttgcagttttaaaattatgttttaaaatggactat |
| | | catatgcttaccgtaacttgaaagtatttcgatttcttggctttatatatcttgtggaaaggac |
| 19 | CMV promoter | gacattgattattgactagttattaatagtaatcaattacggggtcattagttcatagcccatatatggagttccgcgttacataactt |
| | | acggtaaatggcccgcctggctgaccgcccaacgacccccgcccattgacgtcaataatgacgtatgttcccatagtaacgcc |
| | | aatagggactttccattgacgtcaatgggtggagtatttacggtaaactgcccacttggcagtacatcaagtgtatcatatgccaa |
| | | gtacgccccctattgacgtcaatgacggtaaatggcccgcctggcattatgcccagtacatgaccttatgggactttcctacttg |
| | | gcagtacatctacgtattagtcatcgctattaccatggtgatgcggttttggcagtacatcaatgggcgtggatagcggtttgact |
| | | cacggggatttccaagtctccaccccattgacgtcaatgggagtttgttttggcaccaaaatcaacgggactttccaaaatgtcg |
| | | taacaactccgccccattgacgcaaatgggcggtaggcgtgtacggtgggaggtctatataagcagagct |
| 20 | Human | gtgtctagactgcagagggccctgcgtatgagtgcaagtgggttttaggaccaggatgaggcgggggggggtgcctacctg |
| | Synapsin1 | acgaccgaccccgacccactggacaagcacccaacccccattccccaaattgcgcatcccctatcagagagggggagggg |
| | promoter | aaacaggatgcggcgaggcgcgtgcgcactgccagcttcagcaccgcggacagtgccttcgcccccgcctggcggcgcg |
| | | cgccaccgccgcctcagcactgaaggcgcgctgacgtcactcgccggtcccccgcaaactccccttcccggccaccttggt |
| | | cgcgtccgcgccgccgccggcccagccggaccgcaccacgcgaggcgcgagataggggggcacgggcgcgaccatct |
| | | gcgctgcggcgccggcgactcagcgctgcctcagtctgcggtgggcagcggaggagtcgtgtcgtgcctgagagagcgcagt |
| | | cgagaaaccggctaga |
| 21 | gRNA scaffold | gttatagtactctggaaacagaatctactataacaaggcaaaatgccgtgtttatctcgtcaacttgttggcgaga |
| 22 | SV40 nuclear | ccaaagaagaagcggaaggtc |
| | localization | |
| | signal | |
| 23 | Nuclease dead | aagcggaactacatcctgggcctggccatcggcatcaccagcgtgggctacggcatcatcgactacgagacacgggacgtg |
| | SaCas9 DNA | atcgatgccggcgtgcggctgttcaaagaggccaacgtggaaaacaacgagggcaggcggagcaagagaggcgccaga |
| | sequence | aggctgaagcggcggaggcggcatagaatccagagagtgaagaagctgctgttcgactacaacctgctgaccgaccacag |
| | | cgagctgagcggcatcaacccctacgaggccagagtgaagggcctgagccagaagctgagcgaggaagagttctctgcc |
| | | gccctgctgcacctggccaagagaagaggcgtgcacaacgtgaacgaggtggaagaggacaccggcaacgagctgtcca |
| | | ccaaagagcagatcagccggaacagcaaggccctggaagagaaatacgtggccgaactgcagctggaacggctgaagaa |
| | | agacggcgaagtgcggggcagcatcaacagattcaagaccagcgactacgtgaaagaagccaaacagctgctgaaggtg |
| | | cagaaggcctaccaccagctggaccagagcttcatcgacacctacatcgacctgctggaaacccggcggacctactatgag |
| | | ggacctggcgagggcagccccttcggctggaaggacatcaaagaatggtacgagatgctgatgggccactgcacctacttc |
| | | cccgaggaactgcggagcgtgaagtacgcctacaacgccgacctgtacaacgccctgaacgacctgaacaatctcgtgatc |
| | | accagggacgagaacgagaagctggaatattacgagaagttccagatcatcgagaacgtgttcaagcagaagaagaagcc |
| | | caccctgaagcagatcgccaaagaaatcctcgtgaacgaagaggatattaagggctacagagtgaccagcaccggcaagc |
| | | ccgagttcaccaacctgaaggtgtaccacgacatcaaggacattaccgcccggaaagagattattgagaacgccgagctgct |
| | | ggatcagattgccaagatcctgaccatctaccagagcagcgaggacatccaggaagaactgaccaatctgaactccgagctg |
| | | acccaggaagagatcgagcagatctctaatctgaagggctataccggcacccacaacctgagcctgaaggccatcaacctg |
| | | atcctggacgagctgtggcacaccaacgacaaccagatcgctatcttcaaccggctgaagctggtgcccaagaaggtggac |
| | | ctgtcccagcagaaagagatccccaccaccctggtggacgacttcatcctgagccccgtcgtgaagagaagcttcatccaga |
| | | gcatcaaagtgatcaacgccatcatcaagaagtacggcctgcccaacgacatcattatcgagctggcccgcgagaagaactc |
| | | caaggacgcccagaaaatgatcaacgagatgcagaagcggaaccggcagaccaacgagcggatcgaggaaatcatccg |
| | | gaccaccggcaaagagaacgccaagtacctgatcgagaagatcaagctgcacgacatgcaggaaggcaagtgcctgtaca |
| | | gcctggaagccatccctctggaagatctgctgaacaaccccttcaactatgaggtggaccacatcatccccagaagcgtgtcc |
| | | ttcgacaacagcttcaacaacaaggtgctcgtgaagcaggaagaagccagcaagaagggcaaccggaccccattccagta |
| | | cctgagcagcagcgacagcaagatcagctacgaaaccttcaagaagcacatcctgaatctggccaagggcaagggcagaa |
| | | tcagcaagaccaagaaagagtatctgctggaagaacgggacatcaacaggttctccgtgcagaaagacttcatcaaccggaa |
| | | cctggtggataccagatacgccaccagaggcctgatgaacctgctgcggagctacttcagagtgaacaacctggacgtgaaa |
| | | gtgaagtccatcaatggcggcttcaccagctttctgcggcggaagtggaagtttaagaaagagcggaacaaggggtacaag |
| | | caccacgccgaggacgccctgatcattgccaacgccgatttcatcttcaaagagtggaagaaactggacaaggccaaaaaa |
| | | gtgatggaaaaccagatgttcgaggaaaagcaggccgagagcatgcccgagatcgaaaccgagcaggagtacaaagaga |
| | | tcttcatcaccccccaccagatcaagcacattaaggacttcaaggactacaagtacagccaccgggggacaagaagcctaat |
| | | agagagctgattaacgacaccctgtactccacccggaaggacgacaagggcaacaccctgatcgtgaacaatctgaacggc |
| | | ctgtacgacaaggacaatgacaagctgaaaaagctgatcaacaagagccccgaaaagctgctgatgtaccaccacgacccc |
| | | cagacctaccagaaactgaagctgattatggaacagtacggcgacgagaagaatcccctgtacaagtactacgaggaaacc |
| | | gggaactacctgaccaagtactccaaaaaggacaacggccccgtgatcaagaagattaagtattacggcaacaaactgaac |
| | | gcccatctggacatcaccgacgactaccccaacagcagaaacaaggtcgtgaagctgtccctgaagccctacagattcgac |
| | | gtgtacctggacaatggcgtgtacaagttcgtgaccgtgaagaatctggatgtgatcaaaaaagaaaactactacgaagtgaat |
| | | agcaagtgctatgaggaagctaagaagctgaagaagatcagcaaccaggccgagtttatcgcctccttctacaacaacgatct |
| | | gatcaagatcaacggcgagctgtatagagtgatcggcgtgaacaacgacctgctgaaccggatcgaagtgaacatgatcga |
| | | catcacctaccgcgagtacctggaaaacatgaacgacaagaggccccccaggatcattaagacaatcgcctccaagaccca |
| | | gagcattaagaagtacagcacagacattctgggcaacctgtatgaagtgaaatctaagaagcaccctcagatcatcaaaaagg |
| | | gc |
| 24 | Protein | KRNYILGLAIGITSVGYGIIDYETRDVIDAGVRLFKEANVENNEGRRSKRGARRL |
| | sequence | KRRRRHRIQRVKKLLFDYNLLTDHSELSGINPYEARVKGLSQKLSEEEFSAALL |
| | encoded by | HLAKRRGVHNVNEVEEDTGNELSTKEQISRNSKALEEKYVAELQLERLKKDGE |
| | SEQ ID NO: 23 | VRGSINRFKTSDYVKEAKQLLKVQKAYHQLDQSFIDTYIDLLETRRTYYEGPGE |
| | (Nuclease dead | GSPFGWKDIKEWYEMLMGHCTYFPEELRSVKYAYNADLYNALNDLNNLVITR |
| | SaCas9) | DENEKLEYYEKFQIIENVFKQKKKPTLKQIAKEILVNEEDIKGYRVTSTGKPEFT |
| | | NLKVYHDIKDITARKEHIENAELLDQIAKILTIYQSSEDIQEELTNLNSELTQEEIE |
| | | QISNLKGYTGTHNLSLKAINLILDELWHINDNQIAIFNRLKLVPKKVDLSQQKEI |
| | | PTTLVDDFILSPVVKRSFIQSIKVINANIKKYGLPNDIHIELAREKNSKDAQKMINE |
| | | MQKRNRQTNERIEEIIRTTGKENAKYLIEKIKLHDMQEGKCLYSLEAIPLEDLLN |
| | | NPFNYEVDHIIPRSVSFDNSFNNKVLVKQEEASKKGNRTPFQYLSSSDSKISYET |
| | | FKKHILNLAKGKGRISKTKKEYLLEERDINRFSVQKDFINRNLVDTRYATRGLM |
| | | NLLRSYFRVNNLDVKVKSINGGFTSFLRRKWKFKKERNKGYKHHAEDALIIAN |
| | | ADFIFKEWKKLDKAKKVMENQMFEEKQAESMPEIETEQEYKEIFITPHQIKHIK |
| | | DFKDYKYSHRVDKKPNRELINDTLYSTRKDDKGNTLIVNNLNGLYDKDNDKL |
| | | KKLINKSPEKLLMYHHDPQTYQKLKLIMEQYGDEKNPLYKYYEETGNYLTKYS |
| | | KKDNGPVIKKIKYYGNKLNAHLDITDDYPNSRNKVVKLSLKPYRFDVYLDNGV |
| | | YKFVTVKNLDVIKKENYYEVNSKCYEEAKKLKKISNQAEFIASFYNNDLIKING |
| | | ELYRVIGVNNDLLNRIEVNMIDITYREYLENMNDKRPPRIIKTIASKTQSIKKYST |
| | | DILGNLYEVKSKKHPQIIKKG |
| 25 | Nucleoplasmin | aaaaggccggccggccacgaaaaaggccggccaggcaaaaaagaaaaag |
| | nuclear | |
| | localization | |
| | signal | |
| 26 | VP64 DNA | gacgcattggacgattttgatctggatatgctgggaagtgacgccctcgatgattttgaccttgacatgcttggttcggatgccctt |
| | sequence | gatgactttgacctcgacatgctcggcagtgacgcccttgatgatttcgacctggacatgctg |
| 27 | Protein | DALDDFDLDMLGSDALDDFDLDMLGSDALDDFDLDMLGSDALDDFDLDML |
| | sequence | |
| | encoded by | |
| | SEQ ID NO: 26 | |
| | (VP64) | |
| 28 | Bovine growth | ctgtgccttctagttgccagccatctgttgtttgcccctcccccgtgccttccttgaccctggaaggtgccactcccactgtcctttc |
| | hormone | ctaataaaatgaggaaattgcatcgcattgtctgagtaggtgtcattctattctggggggtggggtggggcaggacagcaagg |
| | polyadenylation | gggaggattgggaagagaatagcaggcatgctgggga |
| | signal | |
| 29 | 5′ ITR | cctgcaggcagctgcgcgctcgctcgctcactgaggccgcccgggcaaagcccgggcgtcgggcgacctttggtcgcccg |
| | | gcctcagtgagcgagcgagcgcgcagagagggagtggccaactccatcactaggggttcct |
| 30 | 3′ ITR | aggaacccctagtgatggagttggccactccctctctgcgcgctcgctcgctcactgaggccgggcgaccaaaggtcgccc |
| | | gacgcccgggcggcctcagtgagcgagcgagcgcgcagctgcctgcagg |
| 31 | Mouse Shank3 | aggcgccgctcgaagccggggccgatgcagctaaaccgtgccgccgtcgccgccgccgctgcgcctgcggagccccc |
| | DNA sequence | ggagccgctgtcccccgcgctggccccggccccggccccccccggccccctcccgcgtagcgcggtcggcgggg |
| | (coding region: | tgcggggggtcagggggggccagggcgccgcgcggagtccccgtgcgctcctctctccgccgggaacagtccgggc |
| | bold) | ccgggcgctagcaccgggatggacggccccggggccagcgccgtggtcgtgcgcgtcggcatcccggacctgcaac |
| | | aaacgaagtgcctgcgtctggacccaaccgcgcccgtgtgggccgccaagcagcgtgtgctctgcgccctcaatcata |
| | | gccttcaagacgcgctcaactacgggctattccagcctccctcccggggtcgcgccggcaagttcctggatgaagagc |
| | | ggctcttacaggactacccgcctaacctggacacgcccctgccctatctggagttccgatacaagcggagagtttatgc |
| | | ccagaacctcatagatgacaagcagtttgcaaagctacacacaaaggcaaacctgaagaagttcatggactatgtcc |
| | | agctacacagcacagataaggggcccgcctgctggacaaggggctggaccccaatttccatgaccctgactcagga |
| | | gagtgccctctgagccttgcggcacagttggacaacgccactgacctcctgaaggttctccgcaacggcggtgctcatc |
| | | tggacttccggacccgagatgggctgacagccgtccactgtgctacccgccagcggaacgcaggggcattgacgacc |
| | | ctgctggacctgggggcttcgcctgactacaaggacagccgcggcctgacgcccctgtaccatagtgccctagggggc |
| | | ggggatgccctctgttgcgagctgcttctccatgatcatgcacagctggggaccactgatgagaatggttggcaagag |
| | | atccatcaggcctgtcgctttggacacgtgcagcacctggagcaccttttgttctatggggccaacatgggtgctcaga |
| | | atgcctcgggaaacacagccctgcacatctgtgccctctacaaccaggagagttgcgcgcgcgtcctgcttttccgtgg |
| | | tgccaacaaggacgtccgcaattacaacagccagacagccttccaggtggccattattgcagggaactttgagcttgc |
| | | cgaggtaatcaagacccacaaagactccgatgtcgtaccattcagggaaacccccagctatgcaaagcgacggcgtc |
| | | tggctggcccgagtggcctggcatccccacggcccttacagcgctcagccagtgatatcaacctgaaaggtgatcagc |
| | | ccgcagcttctccagggcccactctccgaagcctccctcatcaactcttgctccagaggcttcaggaggagaaagacc |
| | | gtgacagggatggtgaactggagaatgacatcagcggcccctcagcaggcaggggggccacaacaagatcagccc |
| | | cagtgggcccggcggatccggccccgcgcccggccccggcccggcgtctcccgcgccccccgcgcgcgccccgg |
| | | ggcccgaagcggaaactttacagtgccgtccccggccgcaagttcatcgctgtgaaggcgcacagcccgcagggcga |
| | | gggcgagatcccgctgcaccgcggcgaggccgtgaaggtgctcagcattggggagggcggtttctgggagggaacc |
| | | gtgaagggccgaacaggctggttcccagctgactgtgtggaagaagtgcagatgcgacagtatgacacccggcatga |
| | | aaccagagaggaccggacgaagcgtctcttccgccactacactgtgggttcctatgacagcctcacttcacacagcga |
| | | ttatgtcatcgatgataaggtggctatcctgcagaaaagggaccatgaggggtttggctttgttctccggggagccaaa |
| | | gcagagacccccattgaggagtttacacccacacctgccttccctgcactccaataccttgagtctgtagatgtggaag |
| | | gtgtggcctggagggctggacttcgaactggggacttcctcattgaggtgaacggagtgaatgtcgtgaaggttggac |
| | | Jacaagcaagtggtgggtctcatccgtcagggtggcaaccgcctggtcatgaaggttgtgtctgtgaccaggaaacccg |
| | | aggaggatggtgctcggcgcagagccccaccacccccaaagagggctcccagcaccacgctgaccctgcggtccaa |
| | | gtccatgacggctgagctcgaggaacttgcttccattcggagaagaaaaggggagaagttggatgagatcctggcag |
| | | ttgccgcggagccgacactgaggccggacattgcagatgctgactcgagggcggccactgtcaagcagcggcccacc |
| | | agccggaggatcacccctgctgagatcagctcattgtttgagcgccagggcctcccaggcccagagaagctgccggg |
| | | ctctctgcggaaggggattccacggaccaaatctgtaggggaggatgagaagctggcatccctactggaagggcgct |
| | | tcccacgcagcacgtcaatgcaggacacagtgcgtgaaggtcgaggcattccacccccgccgcagaccgccccgcca |
| | | cccccacccgcgccctactacttcgactccgggccaccccccaccttctcaccgccgccaccaccgggccgggcctatg |
| | | acactgtgcgctccagcttcaaaccaggcctggaggctcgtctgggtgcgggggccgccggcctgtatgatccgagca |
| | | lcgcctctgggcccgctgccctaccctgagcgtcagaagcgtgcgcgctccatgatcatactgcaggactctgcgccag |
| | | aagtgggtgatgtcccccggcctgcgcctgccgccacaccgcctgagcgccccaagcgccggcctcggccgtcaggc |
| | | cctgatagtccctatgccaacctgggcgccttcagtgccagcctcttcgctccgtcgaaaccacagcgcccgcaagagcc |
| | | cgctggtgaagcagcttcaggtggaggacgctcaggagcgcgcggccctggccgtgggtagcccgggaccagtggg |
| | | cggaagctttgcacgagaaccctctccgacgcaccgcgggccccggccaggcagccttgactacagctctggagaag |
| | | gcctaggccttaccttcggcggccctagccctggcccagtcaaggagcggcgcctggaggagcgacgccgttccactg |
| | | tgttcctctctgtgggtgccatcgagggcagccctcccagcgcggatctgccatccctacaaccctcccgctccattgat |
| | | gagcgcctcctggggacaggcgccaccactggccgcgatttgctactcccctcccctgtctctgctctgaagccattggt |
| | | cggtggtcccagccttgggccctcaggctccaccttcatccaccctctcactggcaaacccttggatcctagctcaccctt |
| | | agctcttgctctggctgcccgggagcgggctctggcctcgcaaacaccttcccggtcccccacacctgtgcacagcccc |
| | | gatgctgaccgccctggacccctctttgtggatgtgcaaacccgagactctgagagaggaccgttggcttccccagcct |
| | | tctcccctcggagtccagcgtggattccagtgcctgctcggagagaggcagagaagccccctcgggaagagcggaag |
| | | tcaccagaggacaagaagtccatgatcctcagcgtcttggacacgtccttgcaacggccagctggcctcattgttgtgc |
| | | atgccaccagcaatgggcaggagcccagcaggctgggggctgaagaggagcgccccggtactccggagctggccc |
| | | cagcccccatgcaggcagcagctgtggcagagcccatgccaagcccccgggcccagccccctggcagcatcccagc |
| | | agatcccgggccaggtcaaggcagctcagaggaggagccagagctggtattcgctgtgaacctgccacctgctcagc |
| | | tgtcctccagcgatgaggagaccagagaggagctggcccgcatagggctagtgccaccccctgaagagtttgccaat |
| | | gggatcctgctgaccaccccgcccccagggccgggccccttgcccaccacggtacccagcccggcctcagggaagcc |
| | | cagcagcgagctgccccctgcccctgagtctgcagctgactctggagtagaggaggctgacactcgaagctccagtg |
| | | acccccacctggagaccacaagcaccatttccacagtgtccagcatgtccaccctgagctcggagagtggggaactc |
| | | acggacacccacacctcctttgccgatggacacacttttctactcgagaagccaccagtgcctcccaagcccaagctca |
| | | agtccccgctggggaaggggccggtgaccttcagggacccgctgctgaagcaatcctcggacagtgagctcatggcc |
| | | cagcagcaccatgctgcctctactgggttggcttctgctgctgggcccgcccgccctcgctacctcttccagagaaggtc |
| | | caagctgtggggggaccccgtggagagtcgggggctccctgggcctgaagatgacaaaccaactgtgatcagtgag |
| | | ctcagctcccgtctgcagcagctgaataaagacacacgctccttgggggaggaaccagttggtggcctgggcagcctg |
| | | ctggaccctgctaagaagtcacccattgcagcagctcggctcttcagcagcctcggtgagctgagcaccatctcagcg |
| | | cagcgcagcccggggggcccgggcggaggggcctcctactcggtgcggcccagcggccggtaccccgtggcgagac |
| | | gagccccgagcccagtgaaacccgcatcgctggagcgggggaggggctggggggggcgtgggaggcgcggggc |
| | | ggcccttcggcctcacgcctcccaccatcctcaagtcgtccagcctctccatcccgcacgaacccaaggaagtgcgctt |
| | | cgtggtgcgaagtgtgagtgcgcgcagccgctccccctcaccatctccgctgccctcgccttctcccggctctggcccca |
| | | gtgccggcccgcgtcggccatttcaacagaagcccctgcagctctggagcaagttcgatgtgggcgactggctggaga |
| | | gcatccacttaggcgagcaccgagaccgcttcgaggaccatgagatcgaaggcgcacacctgcctgcgctcaccaag |
| | | gaagacttcgtggagctgggcgtcacacgcgttggccaccgcatgaacatcgagcgtgcgctcaggcagctggatgg |
| | | cagctgacgccccactccctctcctgttcctgctgcgccctgccggcagggcccccacccctactccaggccgcaggctcg |
| | | gctcgccccctaccacggcgcccgggccaggaatgttgcatgaatcgtcctgtttgctgttgcttggagacttgccctgtacatt |
| | | gcttagtgccctcccctgccgctgaaccccacccagcacacagtaagggcgcggaccaggggggctgggtggaaggggg |
| | | tgggggcagggtgctctggcctgaccacttcctccatagctcctggtggccattcttccagagggggaacctagtccagcatg |
| | | cgaggtcaggacacgccttggtgactcggggggaggggggagacattggggttctcgataggggccaaggagccccctgt |
| | | tttacatattttaatccactctatatttggaaagagaaaaggaacaaatatctctgtccgtaatagttcccgccctcttcccctctagt |
| | | ccttcgctggtcccgccacagctgcccagtcttccatctctggcccctcactgccaccccatacagggcaggggacactcca |
| | | gctggcctggggttagccagggtcctggcagcccaccctggggaccccggctcagccccccttccctcgctgagctatagta |
| | | tgccccacccaccctttaggtgctgctcagggggacgggggcaggcattgcctgctgggcactagcagggccaggtggcc |
| | | tgggaaattattgccctggggctgggccccggtaacccaaaccccagccatcatcttcacagggtctctcccacaggagggg |
| | | tctaacctttcccacttcttgggcaactacagcagagaagcctccctgcctcgcaccccaaagactccccaattcctgccctgtg |
| | | tgtgtgtgtgcgccacataatgtgtgtgcacgcctgtgtgcttgtgaaaactgggtgtggctgagcgcatgggtgccctgtgtgt |
| | | gcttgactgtgcagtggtccccaggggctgttctggatggggggaggggcggcagaagcttgcacagggtgcatgcatg |
| | | ggtgtgtgcccgggaaggggccctgtcctgctggtgggtcctgctgttctctcagcctgtcccctcctccccaaacctcaccca |
| | | gcttaagcagggattcttcgtgaatcctttcagcttcgggaggcctcaagggctccccccgcaggcagtaccccttcgggcttc |
| | | taagggaattgtggggaccactaaaatcaggccacaacagcccttggagagaggcaaagactcctgagggtcccctggccc |
| | | ccttactgtgactcctcacactcagcaatgacctgtggggtggggggccctggggcatttttaaacatcgggtttggagtctgg |
| | | actaagctccatccacgtcactcacaagtttctgtttctatttctagctttttttaataaaaaataaataaataaataaatatataaataa |
| | | atatatatatatataaaagacagaaaacaggtgttttcatggcccaggggcttggcacgccggtctgtgcccacctgccccacc |
| | | ctggcccatcggccccattccttagacacagagtcacacccactaaccctctcaccaacagagcaggtcacacacacagcag |
| | | cagtcactgtaacagactgccacatacacagtctcacatttacctgtgggtttttggttctgttcaatttgggtttttaactttacagg |
| | | gtcagttccgcttctcccccccccccttttgtatggagttccatctggggggctttcaaccccctgctccagtcctgaggcctcct |
| | | gaccctgacgttgtgatacaccccacagagatctatgtttcttatattattattattaataataattattataatattatgtaataaatttat |
| | | aagaaatgaaa |
| 32 | Mouse Shank3 | MQLNRAAVAAAAAPAEPPEPLSPALAPAPAPPGPLPRSAVGGALAGGQGGPGR |
| | Protein | RAESPCAPLSAGNSPQPGASTGMDGPGASAVVVRVGIPDLQQTKCLRLDPTAP |
| | sequence (alpha | VWAAKQRVLCALNHSLQDALNYGLFQPPSRGRAGKFLDEERLLQDYPPNLDT |
| | isoform; | PLPYLEFRYKRRVYAQNLIDDKQFAKLHTKANLKKFMDYVQLHSTDKVARLL |
| | Accession No. | DKGLDPNFHDPDSGECPLSLAAQLDNATDLLKVLRNGGAHLDERTRDGLTAV |
| | NP_067398.3) | HCATRQRNAGALTTLLDLGASPDYKDSRGLTPLYHSALGGGDALCCELLLHDH |
| | | AQLGTTDENGWQEIHQACRFGHVQHLEHLLFYGANMGAQNASGNTALHICAL |
| | | YNQESCARVLLFRGANKDVRNYNSQTAFQVAIIAGNFELAEVIKTHKDSDVVP |
| | | FRETPSYAKRRRLAGPSGLASPRPLQRSASDINLKGDQPAASPGPTLRSLPHQLL |
| | | LQRLQEEKDRDRDGELENDISGPSAGRGGHNKISPSGPGGSGPAPGPGPASPAPP |
| | | APPPRGPKRKLYSAVPGRKFIAVKAHSPQGEGEIPLHRGEAVKVLSIGEGGFWE |
| | | GTVKGRTGWFPADCVEEVQMRQYDTRHETREDRTKRLFRHYTVGSYDSLTSH |
| | | SDYVIDDKVAILQKRDHEGFGFVLRGAKAETPIEEFTPTPAFPALQYLESVDVEG |
| | | VAWRAGLRTGDFLIEVNGVNVVKVGHKQVVGLIRQGGNRLVMKVVSVTRKP |
| | | EEDGARRRAPPPPKRAPSTTLTLRSKSMTAELEELASIRRRKGEKLDEILAVAAE |
| | | PTLRPDIADADSRAATVKQRPTSRRITPAEISSLFERQGLPGPEKLPGSLRKGIPR |
| | | TKSVGEDEKLASLLEGRFPRSTSMQDTVREGRGIPPPPQTAPPPPPAPYYFDSGP |
| | | PPTFSPPPPPGRAYDTVRSSFKPGLEARLGAGAAGLYDPSTPLGPLPYPERQKRA |
| | | RSMIILQDSAPEVGDVPRPAPAATPPERPKRRPRPSGPDSPYANLGAFSASLFAPS |
| | | KPQRRKSPLVKQLQVEDAQERAALAVGSPGPVGGSFAREPSPTHRGPRPGSLD |
| | | YSSGEGLGLTFGGPSPGPVKERRLEERRRSTVFLSVGAIEGSPPSADLPSLQPSRS |
| | | IDERLLGTGATTGRDLLLPSPVSALKPLVGGPSLGPSGSTFIHPLTGKPLDPSSPL |
| | | ALALAARERALASQTPSRSPTPVHSPDADRPGPLFVDVQTRDSERGPLASPAFSP |
| | | RSPAWIPVPARREAEKPPREERKSPEDKKSMILSVLDTSLQRPAGLIVVHATSNG |
| | | QEPSRLGAEEERPGTPEAPAPMQAAAVAEPMPSPRAQPPGSIPADPGPGQGSSE |
| | | EEPELVFAVNLPPAQLSSSDEETREELARIGLVPPPEEFANGILLTTPPPGPGPLPT |
| | | TVPSPASGKPSSELPPAPESAADSGVEEADTRSSSDPHLETTSTISTVSSMSTLSSE |
| | | SGELTDTHTSFADGHTFLLEKPPVPPKPKLKSPLGKGPVTFRDPLLKQSSDSELM |
| | | AQQHHAASTGLASAAGPARPRYLPQRRSKLWGDPVESRGLPGPEDDKPTVISE |
| | | LSSRLQQLNKDTRSLGEEPVGGLGSLLDPAKKSPIAAARLESSLGELSTISAQRSP |
| | | GGPGGGASYSVRPSGRYPVARRAPSPVKPASLERVEGLGAGVGGAGRPFGLTP |
| | | PTILKSSSLSIPHEPKEVRFVVRSVSARSRSPSPSPLPSPSPGSGPSAGPRRPFQQKP |
| | | LQLWSKFDVGDWLESIHLGEHRDRFEDHEIEGAHLPALTKEDFVELGVTRVGH |
| | | RMNIERALRQLDGS |
| 33 | Human Shank3 | acacaccgcactggaggctccgctctgcccgctccgggaccccttcctccgccgcacccgccccggtggtccgcggatgcc |
| | DNA sequence | cgcccttgccgctcagccactccccccgcaccgaggcctaggactcccccccccaaccccgtcacagccccccagacccc |
| | (coding region: | gccccgtggctcggcccccgccctccgcacacacctcccgcccccacccgggaccccgcaagtaaccccccagcactg |
| | bold) | gccctgagccctcccggcccccgcctccggcgcagccccctcgccacccccgcttccctcccgtctcaggccccctccccc |
| | | cgccgcccccgcccccggggaaggcaggcgccgagctgagccggggccgatgtagctgagccgcgccgccgccgcc |
| | | gccgccgccgcccctgcggagcccccggagccgctgtcccccgcgccggccccggccccggccccccccggccccct |
| | | cccgcgcagcgcggccgacggggctccggcgggggggaagggggggccggggcgcgcgcggagtccccgggcg |
| | | ctccgttccccggcgcgagcggccccggcccgggccccggcgcggggatggacggccccggggccagcgccgtggt |
| | | cgtgcgcgtcggcatcccggacctgcagcagacgaagtgcctgcgcctggacccggccgcgcccgtgtgggccgcca |
| | | agcagcgcgtgctctgcgccctcaaccacagcctccaggacgcgctcaactatgggcttttccagccgccctcccggg |
| | | gccgcgccggcaagttcctggatgaggagcggctcctgcaggagtacccgcccaacctggacacgcccctgccctac |
| | | ctggagtttcgatacaagcggcgagtttatgcccagaacctcatcgatgataagcagtttgcaaagcttcacacaaag |
| | | gcgaacctgaagaagttcatggactacgtccagctgcatagcacggacaaggtggcacgcctgttggacaaggggct |
| | | ggaccccaacttccatgaccctgactcaggagagtgccccctgagcctcgcagcccagctggacaacgccacggacc |
| | | tgctaaaggtgctgaagaatggtggtgcccacctggacttccgcactcgcgatgggctcactgccgtgcactgtgccac |
| | | acgccagcggaatgcggcagcactgacgaccctgctggacctgggggcttcacctgactacaaggacagccgcggct |
| | | tgacacccctctaccacagcgccctggggggggggatgccctctgctgtgagctgcttctccacgaccacgctcagct |
| | | ggggatcaccgacgagaatggctggcaggagatccaccaggcctgccgctttgggcacgtgcagcatctggagcacc |
| | | tgctgttctatggggcagacatgggggcccagaacgcctcggggaacacagccctgcacatctgtgccctctacaacc |
| | | aggagagctgtgctcgtgtcctgctcttccgtggagctaacagggatgtccgcaactacaacagccagacagccttcc |
| | | aggtggccatcatcgcagggaactttgagcttgcagaggttatcaagacccacaaagactcggatgttgtaccattca |
| | | gggaaacccccagctatgcgaagcggcggcgactggctggccccagtggcttggcatcccctcggcctctgcagcgct |
| | | cagccagcgatatcaacctgaagggggaggcacagccagcagcttctcctggaccctcgctgagaagcctcccccac |
| | | cagctgctgctccagcggctgcaagaggagaaagatcgtgaccgggatgccgaccaggagagcaacatcagtggcc |
| | | ctttagcaggcagggccggccaaagcaagatcagcccgagcgggcccggcggccccggccccgcgcccggccccg |
| | | gccccgcgccccctgcgccccccgcaccgccgccccggggcccgaagcggaaactttacagcgccgtccccggccgc |
| | | aagttcatcgccgtgaaggcgcacagcccgcagggtgaaggcgagatcccgctgcaccgcggcgaggccgtgaagg |
| | | tgctcagcattggggagggcggtttctgggagggaaccgtgaaaggtcgcacgggctggttcccggccgactgcgtg |
| | | gaggaagtgcagatgaggcagcatgacacacggcctgaaacgcgggaggaccggacgaagcggctctttcggcac |
| | | tacacagtgggctcctacgacagcctcacctcacacagcgattatgtcattgatgacaaagtggctgtcctgcagaaac |
| | | gggaccacgagggctttggttttgtgctccggggagccaaagcagagacccccatcgaggagttcacgcccacgcca |
| | | gccttcccggcgctgcagtatctcgagtcggtggacgtggagggtgtggcctggagggccgggctgcgcacgggaga |
| | | cttcctcatcgaggtgaacggggtgaacgtggtgaaggtcggacacaagcaggtggtggctctgattcgccagggtg |
| | | gcaaccgcctcgtcatgaaggttgtgtctgtgacaaggaagccagaagaggacggggctcggcgcagagccccacc |
| | | gccccccaagagggcccccagcaccacactgaccctgcgctccaagtccatgacagctgagctcgaggaacttgcct |
| | | ccattcggagaagaaaaggggagaagctggacgagatgctggcagccgccgcagagccaacgctgcggccagaca |
| | | tcgcagacgcagactccagagccgccaccgtcaaacagaggcccaccagtcggaggatcacacccgccgagattag |
| | | ctcattgtttgaacgccagggcctcccaggcccagagaagctgccgggctccttgcggaaggggattccacggacca |
| | | agtctgtaggggaggacgagaagctggcgtccctgctggaagggcgcttcccgcggagcacctcgatgcaagaccc |
| | | ggtgcgcgagggtcgcggcatcccgcccccgccgcagaccgcgccgcctcccccgcccgcgccctactacttcgactc |
| | | ggggccgcccccggccttctcgccgccgcccccgccgggccgcgcctacgacacggtgcgctccagcttcaagcccg |
| | | gcctggaggcgcgcctgggcgcgggcgctgccggcctgtacgagccgggcgcggccctcggcccgctgccgtatccc |
| | | gagcggcagaagcgcgcgcgctccatgatcatcctgcaggactcggcgcccgagtcgggcgacgcccctcgaccccc |
| | | gcccgcggccaccccgcccgagcgacccaagcgccggccgcggccgcccggccccgacagcccctacgccaacctg |
| | | ggcgccttcagcgccagcctcttcgctccgtccaagccgcagcgccgcaagagccccctggtgaagcagctgcaggt |
| | | ggaggacgcgcaggagcgcgcggccctggccgtgggcagccccggtcccggcggcggcagcttcgcccgcgagcc |
| | | ctccccgacccaccgcggtccgcgcccgggtggcctcgactacggcgcgcgcgatggcccggggctcgcgttcggcg |
| | | gcccgggcccggccaaggaccggcggctggaggagcggcgccgctccactgtgttcctgtccgtgggggccatcgag |
| | | ggcagcgcccccggcgcggatctgccatccctacagccctcccgctccatcgacgagcgcctcctggggaccggcccc |
| | | accgccggccgcgacctgctgctgccctccccggtgtctgccctgaagccgttggtcagcggcccgagcctggggccc |
| | | tcgggttccaccttcatccacccactcaccggcaaacccctggaccccagctcacccctggcccttgccctggctgccc |
| | | gagagcgagctctggcctcccaggcgccctcccggtcccccacacccgtgcacagtcccgacgccgaccgccccgga |
| | | cccctgtttgtggatgtacaggcccgggacccagagcgagggtccctggcttccccggctttctccccacggagcccag |
| | | cctggattcctgtgcctgctcgcagggaggcagagaaggtcccccgggaggagcggaagtcacccgaggacaagaa |
| | | gtccatgatcctcagcgtcctggacacatccctgcagcggccagctggcctcatcgttgtgcacgccaccagcaacgg |
| | | gcaggagcccagcaggctggggggggccgaagaggagcgcccgggcaccccggagttggccccggcccccatgca |
| | | gtcagcggctgtggcagagcccctgcccagcccccgggcccagccccctggtggcaccccggcagacgccgggcca |
| | | ggccagggcagctcagaggaagagccagagctggtgtttgctgtgaacctgccacctgcccagctgtcgtccagcgat |
| | | gaggagaccagggaggagctggcccgaattgggttggtgccaccccctgaagagtttgccaacggggtcctgctggc |
| | | caccccactcgctggcccgggcccctcgcccaccacggtgcccagcccggcctcagggaagcccagcagtgagccac |
| | | cccctgcccctgagtctgcagccgactcctggggggaggaggctgacacacgcagctccagcgacccccacctggag |
| | | accacaagcaccatctccacggtgtccagcatgtccaccttgagctcggagagcggggaactcactgacacccacac |
| | | ctccttcgctgacggacacacttttctactcgagaagccaccagtgcctcccaagcccaagctcaagtccccgctgggg |
| | | aaggggccggtgaccttcagggacccgctgctgaagcagtcctcggacagcgagctcatggcccagcagcaccacg |
| | | ccgcctctgccgggctggcctctgccgccgggcctgcccgccctcgctacctcttccagagaaggtccaagctatgggg |
| | | ggaccccgtggagagccgggggctccctgggcctgaagacgacaaaccaactgtgatcagtgagctcagctcccgcc |
| | | tgcagcagctgaacaaggacacgcgttccctgggggaggaaccagttggtggcctgggcagcctgctggaccctgcc |
| | | aagaagtcgcccatcgcagcagctcggctcttcagcagcctcggtgagctgagctccatttcagcgcagcgcagcccc |
| | | gggggcccgggcggcggggcctcgtactcggtgaggcccagtggccgctaccccgtggcgagacgcgccccgagcc |
| | | cggtgaagcccgcgtcgctggagcgggggggggctgggggcgggcgcggggggcgcagggcggcccttcggcc |
| | | tcacgccccccaccatcctcaagtcgtccagcctctccatcccgcacgagcccaaggaggtgcgcttcgtggtgcgca |
| | | gcgtgagcgcgcgcagtcgctccccctcgccgtcgccgctgccctcgcccgcgtccggccccggccccggcgcccccg |
| | | gcccacgccgacccttccagcagaagccgctgcagctctggagcaagttcgacgtgggcgactggctggagagcatc |
| | | cacctaggcgagcaccgcgaccgcttcgaggaccatgagatagaaggcgcgcacctacccgcgcttaccaaggacg |
| | | acttcgtggagctgggcgtcacgcgcgtgggccaccgcatgaacatcgagcgcgcgctcaggcagctggacggcag |
| | | ctgacgccccacccccactcccgccccggccgtgccctgccggcagggccccccacccccaccccgggccgcgggctc |
| | | ggcctgccccttacgacggcgcccgggccaggaatgttgcatgaatcgtcctgtttgctgttgctcggagactcgccctgtaca |
| | | ttgcttagtgccctcaccggccgcccagcccacccagcgcacagtcaggaagggcgtggaccagggaggctggggggg |
| | | aggtgccgggggggggtgccctagcgtgaccacctccttcgcagctcctggtggccattctcccagagggggaacctagtc |
| | | cagcatgcgaggtcaggacccgccttggtgactcggggggaggggggagacattgggattctcgatgggggccaaggag |
| | | cccccctgttttgcatattttaatccactctatatttggaacgagaaaaggaacaaatatctctgtccgtaatagtttcctctcccctc |
| | | ccttctacttccactggtcccactgcagctgcccagtcttccatctccggcccctcactgccactgccaccccacaacggggca |
| | | ggggacgctccagctggtctggggttggccagggccctagtggcccgccctggggccccagctcggcccctcgcctcgct |
| | | gagctctagtgtgccccaccgacccttcaggtgctgctcgtggtgggaggggcggcaggccgcgggtcctgctgtgcaccc |
| | | gcgggaccagccggcctgggagaccatcggccgggggggatgagggcagggccctgccgctccaccgcagccatcttc |
| | | ctcacagggtctctccccaaggagggggctagcttggtccccatgctcttgggcaactacagcagagaagcctccctgccttg |
| | | gaccccaaagtctcctgtcctgccctttatgtgtgtgggtgaaactgggtgcgtctgagcacgtgggagccgtgtgtgtgcctg |
| | | attactgagtggccaccaggggccgctctggactagcgcggggccgtggaggcgtgcaccgtgtgcatgcgtggggtgtac |
| | | ctgtgagagcaccctgtctcctcttccaaagaaagtcagaggccatcctgcaccctgggtccagctgtttgcccagcctgtcctt |
| | | ccagagcctcacccagcctgagcggggttccctggtgaatccctgctgcttggggaggccccaagggccccttggaggcag |
| | | cgcccccaccttgggcttctgagggcatcatagggggacccctagagtcagttcaccacaggccctggggagagtcaaaga |
| | | cccccgagggtgcccagccccccacactgtgactcctcacactcagcgatgacctgtggggtggggggccctgggacgtttt |
| | | taaacctagggtttggagtctggactaagctccatccacgtcactcacaagtttctgtttatatttctagctttttttaataaaataaaa |
| | | aaaaaaagaaaacagaagttttcacaacccaggggcctggcacgccggtctgtgcctgcccgccccgccctggcccaccg |
| | | gccccactccctgggcacagagtcacacccactcatccttccgccaacagtccaggtcacacagcagcagtcactgtaacag |
| | | actgccacatacacactcggtctcacactcacctgtgggttttggttccgttcaatttgggtttttaactttacagggtcagttccgct |
| | | tcacctccttttgtatggagttccatccggggggtttcaccccctgctccagtcctgaggcctcctgaccctgagttgtgatacg |
| | | ccccacagagatctatgtttcttatattattattattgataataattattataatattattatgtaataaatttataagaaatgaa |
| 34 | Human Shank3 | MQLSRAAAAAAAAPAEPPEPLSPAPAPAPAPPGPLPRSAADGAPAGGKGGPGR |
| | protein | RAESPGAPFPGASGPGPGPGAGMDGPGASAVVVRVGIPDLQQTKCLRLDPAAP |
| | sequence | VWAAKQRVLCALNHSLQDALNYGLFQPPSRGRAGKFLDEERLLQEYPPNLDTP |
| | | LPYLEFRYKRRVYAQNLIDDKQFAKLHTKANLKKFMDYVQLHSTDKVARLLD |
| | | KGLDPNFHDPDSGECPLSLAAQLDNATDLLKVLKNGGAHLDFRTRDGLTAVH |
| | | CATRORNAAALTTLLDLGASPDYKDSRGLTPLYHSALGGGDALCCELLLHDHA |
| | | QLGITDENGWQEIHQACRFGHVQHLEHLLFYGADMGAQNASGNTALHICALY |
| | | NQESCARVLLFRGANRDVRNYNSQTAFQVAIIAGNFELAEVIKTHKDSDVVPFR |
| | | ETPSYAKRRRLAGPSGLASPRPLQRSASDINLKGEAQPAASPGPSLRSLPHQLLL |
| | | QRLQEEKDRDRDADQESNISGPLAGRAGQSKISPSGPGGPGPAPGPGPAPPAPPA |
| | | PPPRGPKRKLYSAVPGRKFIAVKAHSPQGEGEIPLHRGEAVKVLSIGEGGFWEG |
| | | TVKGRTGWFPADCVEEVQMRQHDTRPETREDRTKRLFRHYTVGSYDSLTSHS |
| | | DYVIDDKVAVLQKRDHEGFGFVLRGAKAETPIEEFTPTPAFPALQYLESVDVEG |
| | | VAWRAGLRTGDFLIEVNGVNVVKVGHKQVVALIRQGGNRLVMKVVSVTRKP |
| | | EEDGARRRAPPPPKRAPSTTLTLRSKSMTAELEELASIRRRKGEKLDEMLAAAA |
| | | EPTLRPDIADADSRAATVKQRPTSRRITPAEISSLFERQGLPGPEKLPGSLRKGIP |
| | | RTKSVGEDEKLASLLEGRFPRSTSMQDPVREGRGIPPPPQTAPPPPPAPYYFDSG |
| | | PPPAFSPPPPPGRAYDTVRSSFKPGLEARLGAGAAGLYEPGAALGPLPYPERQK |
| | | RARSMIILQDSAPESGDAPRPPPAATPPERPKRRPRPPGPDSPYANLGAFSASLFA |
| | | PSKPQRRKSPLVKQLQVEDAQERAALAVGSPGPGGGSFAREPSPTHRGPRPGGL |
| | | DYGAGDQPGLAFGGPQPAKDRRLEERRRSTVFLSVGAIEGSAPGADLPSLQPSR |
| | | SIDERLLGTGPTAGRDLLLPSPVSALKPLVSGPSLGPSGSTFIHPLTGKPLDPSSPL |
| | | ALALAARERALASQAPSRSPTPVHSPDADRPGPLFVDVQARDPERGSLASPAFS |
| | | PRSPAWIPVPARREAEKVPREERKSPEDKKSMILSVLDTSLQRPAGLIVVHATSN |
| | | GQEPSRLGGAEEERPGTPELAPAPMQSAAVAEPLPSPRAQPPGGTPADAGPGQG |
| | | SSEEEPELVFAVNLPPAQLSSSDEETREELARIGLVPPPEEFANGVLLATPLAGPG |
| | | PSPTTVPSPASGKPSSEPPPAPESAADSGVEEADTRSSSDPHLETTSTISTVSSMST |
| | | LSSESGELTDTHTSFADGHTFLLEKPPVPPKPKLKSPLGKGPVTFRDPLLKQSSD |
| | | SELMAQQHHAASAGLASAAGPARPRYLFQRRSKLWGDPVESRGLPGPEDDKP |
| | | TVISELSSRLQQLNKDTRSLGEEPVGGLGSLLDPAKKSPIAAARLESSLGELSSIS |
| | | AQRSPGGPQGGASYSVRPSGRYPVARRAPSPVKPASLERVEGLGAGAGGAGRP |
| | | FGLTPPTILKSSSLSIPHEPKEVRFVVRSVSARSRSPSPSPLPSPASGPGPGAPGPR |
| | | RPFQQKPLQLWSKFDVGDWLESIHLGEHRDRFEDHEIEGAHLPALTKDDFVEL |
| | | GVTRVGHRMNIERALRQLDGS* |

### REFERENCES

1. Satterstrom, F. K. et al. Large-Scale Exome Sequencing Study Implicates Both Developmental and Functional Changes in the Neurobiology of Autism. Cell 180, 568-584 e523, doi: 10.1016/j.cell.2019.12.036 (2020).
2. Matharu, N. et al. CRISPR-mediated activation of a promoter or enhancer rescues obesity caused by haploinsufficiency. Science 363, doi: 10.1126/science.aau0629 (2019).
3. Liao, H. K. et al. In Vivo Target Gene Activation via CRISPR/Cas9-Mediated Trans-epigenetic Modulation. Cell 171, 1495-1507 e1415, doi: 10.1016/j.cell.2017.10.025 (2017).
4. Cong. L. & Zhang, F. Genome engineering using CRISPR-Cas9 system. Methods Mol Biol 1239, 197-217, doi: 10.1007/978-1-4939-1862-1_10 (2015).
5. Jinek, M. et al. A programmable dual-RNA-guided DNA endonuclease in adaptive bacterial immunity. Science 337, 816-821, doi: 10.1126/science.1225829 (2012).
6. Konermann, S. et al. Genome-scale transcriptional activation by an engineered CRISPR-Cas9 complex. Nature 517, 583-588, doi: 10.1038/nature 14136 (2015).
7. Perez-Pinera, P. et al. RNA-guided gene activation by CRISPR-Cas9-based transcription factors. Nat Methods 10, 973-976, doi: 10.1038/meth.2600 (2013).
8. Chavez, A. et al. Highly efficient Cas9-mediated transcriptional programming. Nat Methods 12, 326-328, doi: 10.1038/meth.3312 (2015).
9. Hilton, I. B. et al. Epigenome editing by a CRISPR-Cas9-based acetyltransferase activates genes from promoters and enhancers. Nat Biotechnol 33, 510-517, doi: 10.1038/nbt.3199 (2015).
10. Bonaglia, M. C. et al. Disruption of the ProSAP2 gene in a t (12; 22) (q24.1; q13.3) is associated with the 22q13.3 deletion syndrome. Am J Hum Genet 69, 261-268, doi: 10.1086/321293 (2001).
11. Bonaglia, M. C. et al. Identification of a recurrent breakpoint within the SHANK3 gene in the 22q13.3 deletion syndrome. J Med Genet 43, 822-828, doi: 10.1136/jmg.2005.038604 (2006).
12. Naisbitt, S. et al. Shank, a novel family of postsynaptic density proteins that binds to the NMDA receptor/PSD-95/GKAP complex and cortactin. Neuron 23, 569-582, doi: 10.1016/s0896-6273 (00) 80809-0 (1999).
13. Sheng, M. & Kim, E. The Shank family of scaffold proteins. J Cell Sci 113 (Pt 11), 1851-1856 (2000).
14. Durand. C. M. et al. Mutations in the gene encoding the synaptic scaffolding protein SHANK3 are associated with autism spectrum disorders. Nat Genet 39, 25-27, doi: 10.1038/ng 1933 (2007).
15. Moessner, R. et al. Contribution of SHANK3 mutations to autism spectrum disorder. Am J Hum Genet 81, 1289-1297, doi: 10.1086/522590 (2007).
16. Leblond, C. S. et al. Meta-analysis of SHANK Mutations in Autism Spectrum Disorders: a gradient of severity in cognitive impairments. PLOS Genet 10, e1004580, doi: 10.1371/journal.pgen. 1004580 (2014).
17. Bozdagi, O. et al. Haploinsufficiency of the autism-associated Shank3 gene leads to deficits in synaptic function, social interaction, and social communication. Mol Autism 1, 15, doi: 10.1186/2040-2392-1-15 (2010).
18. Guo, B. et al. Anterior cingulate cortex dysfunction underlies social deficits in Shank3 mutant mice. Nat Neurosci 22, 1223-1234, doi: 10.1038/s41593-019-0445-9 (2019).
19. Mei. Y. et al. Adult restoration of Shank3 expression rescues selective autistic-like phenotypes. Nature 530, 481-484, doi: 10.1038/nature 16971 (2016).
20. Peca, J. et al. Shank3 mutant mice display autistic-like behaviours and striatal dysfunction. Nature 472, 437-442, doi: 10.1038/nature09965 (2011).
21. Wang, W. et al. Striatopallidal dysfunction underlies repetitive behavior in Shank3-deficient model of autism. J Clin Invest 127, 1978-1990, doi: 10.1172/JCI87997 (2017).
22. Wang, X. et al. Altered mGluR5-Homer scaffolds and corticostriatal connectivity in a Shank3 complete knockout model of autism. Nat Commun 7, 11459, doi: 10.1038/ncomms11459 (2016).
23. Wang, X. et al. Synaptic dysfunction and abnormal behaviors in mice lacking major isoforms of Shank3. Hum Mol Genet 20, 3093-3108. doi: 10.1093/hmg/ddr212 (2011).
24. Zhou, Y. et al. Mice with Shank3 Mutations Associated with ASD and Schizophrenia Display Both Shared and Distinct Defects. Neuron 89, 147-162, doi: 10.1016/j.neuron.2015.11.023 (2016).
25. Chen, Q. et al. Dysfunction of cortical GABAergic neurons leads to sensory hyper-reactivity in a Shank3 mouse model of ASD. Nat Neurosci 23, 520-532, doi: 10.1038/s41593-020-0598-6 (2020).
26. Han, K. et al. SHANK3 overexpression causes manic-like behaviour with unique pharmacogenetic properties. Nature 503, 72-77, doi: 10.1038/nature 12630 (2013).
27. Livak, K. J. & Schmittgen, T. D. Analysis of relative gene expression data using real-time quantitative PCR and the 2 (−Delta Delta C (T)) Method. Methods 25, 402-408, doi: 10.1006/meth.2001.1262 (2001).
28. Challis, R. C. et al. Systemic AAV vectors for widespread and targeted gene delivery in rodents. Nat Protoc 14, 379-414, doi: 10.1038/s41596-018-0097-3 (2019).
29. Kim, J. Y., Grunke, S. D., Levites, Y., Golde, T. E. & Jankowsky, J. L. Intracerebroventricular viral injection of the neonatal mouse brain for persistent and widespread neuronal transduction. J Vis Exp, 51863, doi: 10.3791/51863 (2014).
30. McCown, T. J. Adeno-Associated Virus (AAV) Vectors in the CNS. Curr Gene Ther 11, 181-188, doi: 10.2174/156652311795684759 (2011).

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

What is claimed is:

1. A construct comprising: a) a Shank3 guide RNA (gRNA) that hybridizes to a nucleic acid sequence of a Shank3 gene; b) a gene editing protein or fragment thereof, wherein the gene editing protein or fragment thereof is a protein of the CRISPR/Cas9 system; and c) a transcriptional activator.

2. The construct of claim 1, wherein the protein of the CRISPR/Cas9 system comprises a nuclease-dead CRISPR/Cas9 protein.

3. The construct of claim 2, wherein the nuclease-dead CRISPR/Cas9 protein is a S. aureus dCas9 protein (dSaCas9).

4. The construct of any one of preceding claims, wherein the transcriptional activator is VP64.

5. The construct of any one of claims 1-4, wherein the Shank3 gRNA hybridizes to a region from about +139 to +159 relative to the transcriptional start site of the Shank3 gene.

6. The construct of any one of claims 1-4, wherein the Shank3gRNA hybridizes to a region from about −64 to −84 relative to the transcriptional start site of the Shank3 gene.

7. The construct of any one of claims 1-4, wherein the Shank3 gRNA hybridizes to a region from about −124 to −104 relative to the transcriptional start site of the Shank3 gene.

8. The construct of any one of claims 1-4, wherein the Shank3 gRNA hybridizes to a region from about −190 to −170 relative to the transcriptional start site of the Shank3 gene.

9. The construct of any one of claims 1-4, wherein the Shank3 gRNA hybridizes to a region from about −228 to −208 relative to the transcriptional start site of the Shank3 gene.

10. The construct of any one of claims 1-4, wherein the Shank3 gRNA hybridizes to a region from about −303 to −283 relative to the transcriptional start site of the Shank3 gene.

11. The construct of any one of claims 1-4, wherein the Shank3 gRNA hybridizes to a region from about −359 to −339 relative to the transcriptional start site of the construct.

12. The construct of any one of claims 1-11, wherein the Shank3 gRNA sequence 12, comprises a nucleic acid sequence of any one of SEQ ID NOs: 1-7.

13. The construct of claim 12, wherein the Shank3 gRNA sequence comprises SEQ ID NO: 3.

14. The construct of any one of preceding claims further comprising at least one promoter.

15. The construct of claim 14, wherein the at least one promoter is a CMV promoter, a U6 promoter, and/or a human Synapsin 1 (hSyn1) promoter.

16. The construct of claim 14 or 15, wherein the construct comprises a first promoter that drives expression of the gRNA and a second promoter that drives expression of the protein of the CRISPR/Cas9 system.

17. The construct of claim 15, wherein the first promoter is the U6 promoter and the second promoter is the CMV promoter.

18. The construct of claim 15, wherein the first promoter is the U6 promoter and the second promoter is the hSyn1 promoter.

19. The construct of any one of preceding claims, wherein the construct further comprises adeno-associated virus (AAV) inverted terminal repeats (ITRs).

20. The construct of claim 19, wherein the ITRs are adeno-associated virus ITRs of a serotype selected from the group consisting of AAVI ITR, AAV2 ITR, AAV3 ITR, AAV4 ITR, AAV5 ITR, AAV6 ITR, and AAV9 ITR.

21. The construct of claim 20, wherein the ITRs are AAV2 and/or AAV9 ITRs.

22. The construct of claim 21, wherein the ITR comprises a sequence that is at least 90% identical to SEQ ID NO: 29 or 30.

23. The construct of claim 22, wherein the ITR comprises the sequence of SEQ ID NO: 29 or 30.

24. The construct of any one of claims 3-23, wherein the construct further comprises a polyadenylation signal positioned between the dSaCas9 and the 3′ ITR.

25. The construct of claim 24, wherein the polyadenylation signal is a bovine growth hormone (bGH) polyadenylation signal.

26. The construct of any one of preceding claims, wherein the construct is capable of upregulating Shank3 gene expression in vivo.

27. The construct of any one of preceding claims, wherein the construct is capable of upregulating Shank3 gene expression at least to the level of Shank3 gene expression in a control subject.

28. The construct of claim 27, wherein the control subject does not have disruption of the Shank3 gene.

29. The construct of any one of preceding claims, wherein the construct comprises a sequence that is at least 90% identical to SEQ ID NO: 17.

30. The construct of claim 29, wherein the construct comprises the sequence of SEQ ID NO: 17.

31. A vector comprising the construct of any one of the preceding claims.

32. The vector of claim 31, wherein the vector is a viral vector.

33. The vector of claim 32, wherein the viral vector is an AAV vector.

34. A recombinant adeno-associated virus (rAAV) comprising:
(a) the construct of any one of claims 1-30; and
(b) at least one AAV capsid protein.

35. The rAAV of claim 34, wherein the capsid protein is of a serotype selected from AAVI, AAV2, AAV3, AAV4, AAV5, AAV6, AAV6.2, AAV7, AAV8, AAVrh.8, AAV9, AAVrh.10, AAVrh39, and AAVrh.43.

36. The rAAV of claim 35, wherein the serotype is AAV2.

37. The rAAV of claim 35, wherein the serotype is AAV9.

38. A method of activating Shank3 gene expression in a subject in need thereof comprising administering the construct of any one of claims 1-26.

39. A method of upregulating Shank3 gene expression comprising administering the vector of any one of claims 31-33 or the rAAV of any one of claims 34-37 to a subject in need thereof.

40. A method comprising administering the vector of any one of claims 31-33 or the rAAV of any one of claims 34-37 to a subject in need thereof.

41. The method of claim 39 or 40, wherein the vector or rAAV is administered intracerebroventricularly.

42. The method of claim 39 or 40, wherein the vector or rAAV is delivered to the brain of the subject.

43. The method of claim 42, wherein the vector or rAAV is delivered to the cortex, striatum, cerebellum, brain stem, and/or thalamus of the subject.

44. The method of claim 39 or 40, wherein the vector or rAAV is administered intravenously.

45. The method of claim 39 or 40, wherein the subject is a human subject.

46. The method of claim 45, wherein the human subject is an adult.

47. The method of claim 45, wherein the human subject is not an adult.

48. The method of claim 47, wherein the human subject is a newborn.

49. The method of claim 47, wherein the human subject is a child older than 1 year old.

50. The method of any one of claims 38-49, wherein the subject has, is suspected of having, or is at risk of having, a neurodevelopmental disorder.

51. The method of any one of claims 38-49, wherein the subject has, is suspected of having, or is at risk of having, an autism spectrum disorder (ASD).

52. The method of any one of claims 38-49, wherein the subject exhibits one or more symptoms of an ASD.

53. The method of any one of claims 38-49, wherein the subject has, is suspected of having, or is at risk of having, Phelan-McDermid syndrome.

54. The method of any one of claims 38-53, wherein the subject exhibits one or more of: developmental delay, intellectual disability (ID), sleep disturbance, hypotonia, lack of speech, or language delay.

55. The method of any one of claims 38-54, wherein the subject has, is suspected of having, or is at risk of having, reduced expression of the Shank3 gene relative to a control subject.

56. The method of claim 55, wherein the control subject is a subject that does not have, is not suspected of having, or is not at risk of having, a neurodevelopmental disorder, an autism spectrum disorder (ASD), and/or Phelan-McDermid syndrome.

57. The method of claim 55, wherein reduced expression of the Shank3 gene is caused by disruption of at least one copy of the Shank3 gene.

58. The method of claim 57, wherein disruption of the Shank3 gene comprises a deletion or inactivation in at least one copy of the Shank3 gene.

59. The method of claim 57, wherein disruption of the Shank3 gene comprises one or more mutations within at least one copy of the Shank3 gene.

60. A method for upregulating expression of Shank3 in a subject in need thereof, comprising activating expression of an endogenous Shank3 gene using CRISPR-mediated transcription activation.

61. A method of treating a subject having a neurodevelopmental disorder, the method comprising administering to the subject an effective amount of a composition comprising an AAV vector, wherein the AAV vector comprises a construct comprising a) a Shank3 guide RNA (gRNA) that hybridizes to a nucleic acid sequence of a Shank3 gene; b) a gene editing protein or fragment thereof, wherein the gene editing protein or fragment thereof is a protein of the CRISPR/Cas9 system; and c) a transcriptional activator.

62. A method of treating a subject having an autism spectrum disorder (ASD), the method comprising administering to the subject an effective amount of a composition comprising an AAV vector, wherein the AAV vector comprises a construct comprising a) a Shank3 guide RNA (gRNA) that hybridizes to a nucleic acid sequence of a Shank3 gene; b) a gene editing protein or fragment thereof, wherein the gene editing protein or fragment thereof is a protein of the CRISPR/Cas9 system; and c) a transcriptional activator.

63. A method of treating a subject having Phelan-McDermid syndrome, the method comprising administering to the subject an effective amount of a composition comprising an AAV vector, wherein the AAV vector comprises a construct comprising a) a Shank3 guide RNA (gRNA) that hybridizes to a nucleic acid sequence of a Shank3 gene; b) a gene editing protein or fragment thereof, wherein the gene editing protein or fragment thereof is a protein of the CRISPR/Cas9 system; and c) a transcriptional activator.

64. The method of claim 60, wherein the CRISPR-mediated transcription activation comprises administering to the subject a construct that comprises: (i) a Shank3 guide RNA (gRNA) that hybridizes to a nucleic acid sequence of a Shank3 gene; (ii) a protein of the CRISPR/Cas9 system; and (iii) a transcriptional activator.

65. The method of any one of claims 61-64, wherein the protein of the CRISPR/Cas9 system comprises a nuclease-dead CRISPR/Cas9 protein.

66. The method of claim 65, wherein the protein of the CRISPR/Cas9 system is a S. aureus dCas9 protein (dSaCas9).

67. The method of any one of claims 61-66, wherein the transcriptional activator is VP64.

68. The method of any one of claims 61-67, wherein the Shank3 gRNA hybridizes to a region from about +139 to +159 relative to the transcriptional start site of the Shank3 gene.

69. The method of any one of claims 61-67, wherein the Shank3 gRNA hybridizes to a region from about −64 to −84 relative to the transcriptional start site of the Shank3 gene.

70. The method of any one of claims 61-67, wherein the Shank3 gRNA hybridizes to a region from about −124 to −104 relative to the transcriptional start site of the Shank3 gene.

71. The method of any one of claims 61-67, wherein the Shank3 gRNA hybridizes to a region from about −190 to −170 relative to the transcriptional start site of the Shank3 gene.

72. The method of any one of claims 61-67, wherein the Shank3 gRNA hybridizes to a region from about −228 to −208 relative to the transcriptional start site of the Shank3 gene.

73. The method of any one of claims 61-67, wherein the Shank3 gRNA hybridizes to a region from about −303 to −283 relative to the transcriptional start site of the Shank3 gene.

74. The method of any one of claims 61-67, wherein the Shank3 gRNA hybridizes to a region from about −359 to −339 relative to the transcriptional start site of the construct.

75. The method of any one of claims 61-67, wherein the Shank3 gRNA sequence comprises a nucleic acid sequence of any one of SEQ ID NOs: 1-7.

76. The method of claim 75, wherein the Shank3 gRNA sequence comprises SEQ ID NO: 3.

77. The method of any one of claims 61-76, wherein the construct further comprises at least one promoter.

78. The method of claim 77, wherein at least one promoter is a CMV, a U6 promoter, and/or a human Synapsin 1 (hSyn1) promoter.

79. The method of claim 77 or 78, wherein the construct comprises a first promoter that drives expression of the gRNA and a second promoter that drives expression of the protein of the CRISPR/Cas9 system.

80. The method of claim 79, wherein the first promoter is the U6 promoter and the second promoter is the CMV promoter.

81. The method of claim 79, wherein the first promoter is the U6 promoter and the second promoter is the hSyn1 promoter.

82. The method of any claims 61-81, wherein the construct further comprises adeno-associated virus (AAV) inverted terminal repeats (ITRs).

83. The method of claim 82, wherein the ITRs are adeno-associated virus ITRs of a serotype selected from the group consisting of AAVI ITR, AAV2 ITR, AAV3 ITR, AAV4 ITR, AAV5 ITR, AAV6 ITR, and AAV9 ITR.

84. The method of claim 83, wherein the ITRs are AAV2 and/or AAV9 ITRs.

85. The method of claim 84, wherein the ITR comprises a sequence that is at least 90% identical to SEQ ID NO: 29 or 30.

86. The method of claim 85, wherein the ITR comprises the sequence of SEQ ID NO: 29 or 30.

87. The method of any one of claims 66-80, wherein the construct further comprises a polyadenylation signal positioned between the dSaCas9 and the 3′ ITR.

88. The method of claim 87, wherein the polyadenylation signal is a bovine growth hormone (bGH) polyadenylation signal.

89. The method of any one of claims 60-88, wherein the subject is a human subject.

90. The method of claim 89, wherein the human subject is an adult.

91. The method of claim 89, wherein the human subject is not an adult.

92. The method of claim 91, wherein the human subject is a newborn.

93. The method of claim 91 wherein the human subject is a child older than 1 year old.

94. The method of any one of claims 60-93, wherein the subject has, is suspected of having, or is at risk of having, a neurodevelopmental disorder.

95. The method of any one of claims 60-93, wherein the subject has, is suspected of having, or is at risk of having, an autism spectrum disorder (ASD).

96. The method of any one of claims 60-93, wherein the subject exhibits one or more symptoms of an ASD.

97. The method of any one of claims 60-93, wherein the subject has, is suspected of having, or is at risk of having, Phelan-McDermid syndrome.

98. The method of any one of claims 60-97, wherein the subject exhibits one or more of: developmental delay, intellectual disability (ID), sleep disturbance, hypotonia, lack of speech, or language delay.

99. The method of any one of claims 60-98, wherein the subject has, is suspected of having, or is at risk of having, reduced expression of the Shank3 gene relative to a control subject.

100. The method of claim 99, wherein the control subject is a subject that does not have, is not suspected of having, or is not at risk of having, a neurodevelopmental disorder, an autism spectrum disorder (ASD), and/or Phelan-McDermid syndrome.

101. The method of claim 99, wherein reduced expression of the Shank3 gene is caused by disruption of at least one copy of the Shank3 gene.

102. The method of claim 101, wherein disruption of the Shank3 gene comprises a deletion or inactivation in at least one copy of the Shank3 gene.

103. The method of claim 101, wherein disruption of the Shank3 gene comprises one or more mutations within at least one copy of the Shank3 gene.

104. The method of any one of claims 61-103, wherein the subject has improved sleep efficiency after being administered to an effective amount of the composition.

105. The method of any one of claims 61-104, wherein the composition includes a pharmaceutically acceptable carrier.
